(19) United States
(12) Patent Application Publication
(10) Pub. No.: US 2023/0339854 A1
(43) Pub. Date: Oct. 26, 2023

(54) Negative Allosteric Modulation of GluN3-Containing N-Methyl-D-Aspartate Receptors

(71) Applicant: Emory University, Atlanta, GA (US)

(72) Inventors: Dennis C. Liotta, Atlanta, GA (US); Stephen Traynelis, Decatur, GA (US); Hongjie Yuan, Tucker, GA (US); Matthew Epplin, Atlanta, GA (US); Samantha Summer, Stone Mountain, GA (US); David Menaldino, Atlanta, GA (US); Leon Jacobs, Atlanta, GA (US); Adam McCallum, Atlanta, GA (US)

(21) Appl. No.: 17/796,387

(22) PCT Filed: Feb. 1, 2021
(86) PCT No.: PCT/US21/16074

§ 371 Date: Jul. 29, 2022

Related U.S. Application Data
(60) Provisional application No. 62/968,005, filed on Jan. 30, 2020.

(51) Int. Cl. C07D 209/46; C07D 217/24; C07D 401/06; C07D 401/12

(52) U.S. Cl. CPC C07D 209/46; C07D 217/24; C07D 401/06; C07D 401/12

(57) ABSTRACT

Disclosed are negative allosteric modulators of GluN3-containing NMDA receptors. In general, these compounds are highly selective for GluN3 (such as GluN3A and/or GluN3B) over GluN1 and/or GluN2. They can function as non-competitive antagonists with activity that is independent of membrane potential, glycine concentration, and extracellular pH. Also disclosed are pharmaceutical formulations of the negative allosteric modulators. These compounds can be used to enhance synaptic function and/or treating a neurological condition or disorder. Exemplary neurological conditions or disorders include, but are not limited to, major mental disorders, conditions that involve basal ganglia or altered dopamine, substance abuse/addiction or predisposition to substance abuse/addiction, pain disorders, developmental delay or situations with impaired learning, memory, and/or cognition, acute neuronal or glial injuries, and circuit disorders.

### CROSS REFERENCE TO RELATED APPLICATIONS

This application claims the benefit of U.S. Provisional Application No. 62/968,005 filed Jan. 30, 2020. The entirety of this application is hereby incorporated by reference for all purposes.

### STATEMENT REGARDING FEDERALLY SPONSORED RESEARCH OR DEVELOPMENT

This invention was made with government support under NS111619 and HD082373 awarded by the National Institutes of Health. The government has certain rights in the invention.

### TECHNICAL FIELD

The present disclosure relates to negative allosteric modulators of GluN3-containing N-methyl-D-aspartate (NMDA) receptors and derivatives thereof, pharmaceutical compositions and uses related thereto.

### BACKGROUND

N-methyl-D-aspartate (NMDA) receptors are ligand-gated ion channels that belong to the family of ionotropic glutamate receptors. NMDA receptors that contain GluN1 and GluN2 subunits have been extensively studied over the past several decades, and many of the structural and functional properties are now well understood. In stark contrast to GluN1/GluN2 NMDA receptors, many key properties of GluN3-containing NMDA receptors remain elusive.

NMDA receptors composed of two GluN1 and two GluN3 subunits (GluN1/GluN3) have been consistently demonstrated in heterologous expression systems (e.g., Xenopus oocytes and HEK cells). NMDA receptors composed of GluN1, GluN2, and GluN3 subunits (GluN1/GluN2/GluN3) have also been suggested to exist in some expression systems, but the function and subunit stoichiometry of GluN1/GluN2/GluN3 receptors have not been resolved. GluN1 and GluN3A both bind glycine (and D-serine), and GluN1/GluN3 receptors only require glycine for activation, but strongly desensitize following glycine exposure. GluN1/GluN2 receptors have high Ca²⁺ permeability and are blocked by extracellular Mg′ at resting membrane potentials, while recombinant GluN1/GluN3 receptors are impermeable to Ca²⁺ and insensitive to Mg²⁺ block. Thus, GluN3 subunits endow NMDA receptors with unique functional properties. In addition, the GluN3A subunit has been suggested to influence synaptic development, plasticity and place aversion conditioning. Some reports provide evidence that GluN3 subunits can be neuroprotective. These properties of GluN3 subunits raise the possibility that these subunits could be new targets for therapeutic strategies to treat neurological diseases. In this regard, pharmacological agents that are selective for GluN3 subunits are needed to establish proof-of-concept for therapeutic utility, and potentially serve as a starting point for drug development.

GluN1/GluN3 receptors have seemingly cryptic activation properties, since agonist binding to GluN1 triggers strong desensitization of GluN1/GluN3 receptors, whereas agonist binding to GluN3 mediates activation. Furthermore, glycine appears to bind GluN1 with high potency (EC₅₀˜0.1-1 μM) and GluN3A with low potency (EC₅₀˜10-60 μM). Consistent with this idea, mutations within the GluN1 agonist binding pocket that block glycine binding can prevent the desensitization of GluN1/GluN3 receptors that is mediated by glycine binding to GluN1. Recent data with glycine site antagonists further supports this conclusion and shows that selective block of GluN1 can enhance the glycine response from GluN1/GluN3 receptors. For example, CGP-78608, which is a glycine-site antagonist that is highly selective for GluN1 over GluN3 subunits, can dramatically enhance activation of GluN1/GluN3 receptors. However, CGP-78608 blocks GluN1/GluN2 NMDA receptors through its action at the GluN1 glycine site, which limits its utility as a tool to evaluate the contribution of GluN3 to neurological functions. Indeed, there are currently no reliable pharmacological means to selectively inhibit or potentiate GluN3-containing receptors, apart from TK13, TK30, and TK80 antagonists, which show relatively modest preference (˜5- to 10-fold) for GluN1/GluN3 over GluN1/GluN2 receptors.

Evaluation of potential extracellular modulators has revealed strong regulation by both protons and Zn²⁺. Moreover, the recent study using CGP-78608 to prevent GluN1/GluN3 desensitization has revealed a remarkable sensitivity to redox reagents and Zn²⁺, suggesting that glycine auto-inhibition may occur in vivo to allow the GluN1/GluN3 receptor to respond to other extracellular conditions. In addition to uncertainty that has surrounded GluN1/GluN3 receptors, there is also a lack of data with which to understand potential triheteromeric GluN1/GluN2/GluN3 receptors. While early studies have suggested this stoichiometry to reflect a major role of GluN3, there have been few successful attempts to confirm early findings, and thus there remains a question about the potential role of GluN3 in NMDA receptors. In this regard, the identification of a selective inhibitor of GluN3 activity could provide a valuable tool with which to evaluate the potential role of this receptor class in various neuronal functions.

Therefore, there is an urgent need for compounds with improved binding specificity toward GluN3. In particular, there is a need for GluN3-selective negative allosteric modulators.

### SUMMARY

It is an object of the invention to provide compounds with improved binding specificity toward GluN3, such as GluN3A and/or GluN3B.

It is another object of the invention to provide pharmaceutically acceptable formulations of the compounds disclosed herein.

It is yet another object of the invention to provide improved therapies involving the compounds disclosed herein and their pharmaceutically acceptable formulations thereof.

Disclosed are compounds with improved binding specificity toward GluN3, such as GluN3A and/or GluN3B. These compounds can specifically inhibit GluN3, without perturbing GluN1 and/or GluN2.

In some embodiments, the disclosed compounds have a structure of Formula I, an enantiomer or diastereomer thereof, or a pharmaceutically acceptable salt thereof,

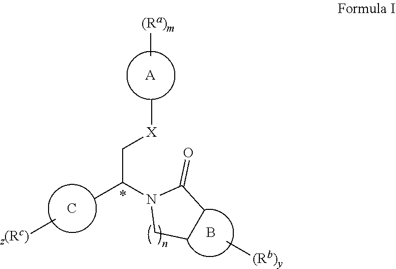

wherein rings A, B and C are individually and independently five- or six-membered aryl, heteroaryl, carbocycle, or heterocycle;
wherein X is O, S, or NH;
wherein n is 1, 2 or 3;
wherein m, y, and z are individually and independently 0, 1, 2, 3, or 4; and
wherein R^{a}, R^{b}, and R^{c}, in each occurrence, are individually and independently selected from hydrogen, deuterium, halogen, nitro, cyano, hydroxyl, trifluoromethoxy, trifluoromethyl, alkylsilyl (such as trimethylsilyl, methyl(methyl)(ethyl)silyl, triethylsilyl, triisopropylsilyl, methyl(methyl)(tert-butyl)silyl, methyl(methyl)(isobutyl)silyl), formyl, carboxyl, mercapto, sulfamoyl, alkyl (such as methyl, ethyl, isopropyl, tert-butyl), alkyloxy (such as methoxy, ethoxy), acyl (such as acetyl), acyloxy (such as acetoxy), amino, alkylamino (such as methylamino, ethylamino, dimethylamino, diethylamino, N-methyl-N-ethylamino), acylamino (acetylamino), carbamoyl, N-alkylcarbamoyl (such as N-methylcarbamoyl, N-ethylcarbamoyl, N,N-dimethylcarbamoyl, N,N-diethylcarbamoyl, N-methyl-N-ethylcarbamoyl), alkylthio (such as methylthio, ethylthio), alkylsulfinyl (such as methylsulfinyl, ethylsulfinyl), alkylsulfonyl (such as mesyl, ethylsulfonyl), alkyloxycarbonyl (such as methoxycarbonyl, ethoxycarbonyl), N-alkylsulfamoyl (such as N-methylsulfamoyl, N-ethylsulfamoyl, N,N-dimethylsulfamoyl, N,N-diethylsulfamoyl, N-methyl-N-ethylsulfamoyl), arylalkyl (such as benzyl), arylcarbonyl (such as benzoyl), heteroalkyl, carbocyclyl, heterocyclyl, aryl, and heteroaryl.

Optionally, R^{a}, R^{b}, and R^{c}, in each occurrence, can be individually and independently substituted by one or more sub stituents.

In some embodiments, X is O.

In some embodiments, rings A, B and C are individually and independently five- or six-membered aryl or heteroaryl. Optionally, ring A is phenyl or pyridinyl (such as 2-pyridinyl). Optionally, ring B is phenyl. Optionally, ring C is phenyl.

In some embodiments, n is 1 or 2. In some embodiments, m is 1. In some embodiments, y is 0 or 1. In some embodiments, z is 0 or 1.

In some embodiments, R^{a}, R^{b}, and R^{c}, in each occurrence, are individually and independently selected from hydrogen, deuterium, halogen, nitro, cyano, hydroxyl, trifluoromethoxy, trifluoromethyl, trimethylsilyl, formyl, carboxyl, carbamoyl, mercapto, sulfamoyl, alkyl, alkoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethyl carbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, benzyl, benzoyl, carbocyclyl, aryl, and heterocyclyl. Optionally, R^{a}, R^{b}, and R^{c}, in each occurrence, can be individually and independently substituted by one or more substituents.

In some embodiments, R^{a}, R^{b}, and R^{c}, in each occurrence, are individually and independently selected from hydrogen, halogen, hydroxyl, methyl, trifluoromethyl, trifluoromethoxy, methoxy, ethoxy, and isopropoxy.

Also disclosed are compositions containing a compound described herein, wherein the compound is in greater than 60%, 70%, 80%, 90%, 95%, or 98% enantiomeric excess with respect to the stereocenter labeled by the “*” sign in Formula I.

In some embodiments, the compound in its corresponding composition is in greater than 95% enantiomeric excess for the configuration depicted below:

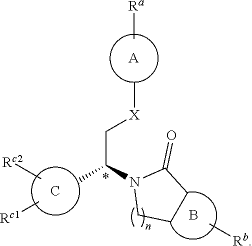

In some embodiments, the compound in its corresponding composition is in greater than 95% enantiomeric excess for the configuration depicted below:

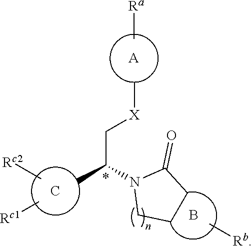

Also disclosed are pharmaceutical formulations of the disclosed compounds or compositions. In general, the pharmaceutical formulations also contain a pharmaceutically acceptable carrier. In some embodiments, the pharmaceutical formulations are in the form of tablet, capsule, pill, gel, cream, granule, solution, suspension, emulsion, or nanoparticulate formulation. In some embodiments, the pharmaceutical formulations are oral formulations.

This disclosure also relates to (1) the compounds disclosed herein for treatment of a neurological condition or disorder disclosed herein or use as a medicament, (2) the compounds disclosed herein for use in the treatment of a neurological condition or disorder disclosed herein, or (3) the compounds disclosed herein for the manufacture of a medicament for treatment of a neurological condition or disorder disclosed herein.

This disclosure also provides methods of enhancing synaptic function and/or treating a neurological condition or disorder in a subject in need thereof. The method includes administering an effective amount of a compound disclosed herein to the subject. In some embodiments, the neurological condition or disorder is mediated by GluN3-containing NMDA receptors.

### BRIEF DESCRIPTION OF THE DRAWINGS

The present disclosure will now be described in more detail with reference to the following figures, in which:

FIGS. 1A-1D show the evaluation of EU1180-438 as a selective GluN1/GluN3 receptor antagonist.

FIG. 1A is a representative two-electrode voltage-clamp recording of EU1180-438 inhibition of recombinant GluN1^{FA,TL}/GluN3A receptor responses activated by 1000 μM glycine.

FIG. 1B shows concentration-response curves for inhibition of GluN1_{FA,TL}/GluN3A receptor responses activated by 1000 μM glycine by enantiomers and racemate of EU1180-438 ((−)-EU1180-438 IC₅₀ 13 μM, Slope 1.4, minimum 16%; (+)-EU1180-438 IC₅₀ 7.1 μM, Slope 1.2, minimum 4%; (±)-EU1180-438 IC₅₀ 3.5 μM, Slope 1.24, minimum 18%). Data are mean±SEM of 18-22 oocytes.

FIG. 1C shows concentration-response relationship for EU1180-438 in 500 nM CGP-78608 plus 100 μM glycine at human GluN1/GluN3A and rat GluN1/GluN3B receptors (IC₅₀ 1.8 μM, Slope 1.5, minimum 15% and IC₅₀ 2.2 μM, Slope 1.5, minimum 7%). Data are mean±SEM of 8-9 oocytes.

FIG. 1D shows current ratio for receptor responses to agonists in the presence of 20 μM EU1180-438 to agonist responses in vehicle. GluN1 was co-expressed with GluN2A-D and activated by 100 μM glutamate/100 μM glycine, GluA1-4 and GluK2 were activated by 100 μM glutamate, GABAA and GABAC were activated by 100 μM GABA, Glycine al was activated by 100 μM glycine, P2X purinergic receptors were activated by 9 μM ATP, α1β1γδ nicotinic acetylcholine receptors were activated by 1 μM acetylcholine, α4 ρ2 nicotinic acetylcholine receptors were activated by 10 μM acetylcholine, and α7-nicotinic acetylcholine receptors were activated by 300 μM acetylcholine. The numbers of oocytes recorded are in parentheses.

FIGS. 2A-2I show modulation of native GluN3A-containing NMDA receptors.

FIG. 2A is representative recordings of current responses from GluN3A-containing NMDA receptors in hippocampal CA1 pyramidal cells from acute mouse brain slices (P8-15).

The responses were activated by pressure-induced application of 10 mM glycine to the neuronal cell soma from wild type (WT) and GluN3A-deficient (3A-KO) mice in the absence or presence of bath-applied 1 μM CGP-78608 (CGP). The current responses from 3A-KO mice in the absence and presence of CGP are nearly identical.

FIG. 2B shows averaged development of glycine responses from WT and 3A-KO mice following addition of CGP-78608 to the recording solution.

FIG. 2C is a bar graph summarizing the glycine-activated responses from GluN3A-containing NMDA receptors revealed by CGP-78608 in WT and 3A-KO mice.

FIG. 2D shows modulation data of GluN3A-containing NMDA receptors in the presence of 1 μM CGP. The GluN3A-containing NMDA receptor-mediated responses were inhibited significantly by 30 μM EU1180-438. The two traces on the top are very similar.

FIG. 2E shows glycine responses over time in the presence of 1 μM CGP in the vehicle-treated group and the EU1180-438-treated group.

FIG. 2F is a bar graph showing the magnitudes of glycine responses in the presence of 1 μM CGP in the vehicle-treated group and the EU1180-438-treated group.

FIG. 2G shows modulation data of GluN1/GluN2 NMDA receptors. EU1180-438 (30 μM) did not affect current responses from GluN1/GluN2 NMDA receptors in hippocampal CA1 pyramidal cells from acute mouse brain slices (P8-15). The responses were activated by pressure-induced application of 0.5 mM NMDA to the neuronal cell soma from WT mice.

FIG. 2H shows NMDA responses over time in the vehicle-treated group and the EU1180-438-treated group.

FIG. 2I is a bar graph showing the magnitudes of NMDA responses in the vehicle-treated group and the EU1180-438-treated group.

FIGS. 3A-3I show EU1180-438 inhibition of GluN1/GluN3A current responses.

FIG. 3A shows representative recordings of the inhibition of GluN1^{FA,TL}/GluN3 responses by 10 μM EU1180-438 in the presence of 1 mM or 3 mM glycine (representative responses from n=10 oocytes).

FIG. 3B shows effects of 4 μM EU1180-438 on glycine concentration-response curve, normalized by current at +40 mV (mean±SEM, n=12 oocytes).

FIG. 3C shows effects of 4 μM EU1180-438 on current-voltage relationship.

FIG. 3D shows that there was no effect of holding voltage on EU1180-438 inhibition (n=12), suggesting that the inhibition does not reflect voltage-dependent channel block.

FIG. 3E shows a composite concentration-response curve for inhibition of GluN1^{FA,TL}/GluN3A at pH 6.8 (n=8 oocytes), shown together with the corresponding inhibition data at pH 7.4 from FIG. 1C (dashed line).

FIG. 3F shows representative neuronal current responses from GluN1/GluN3A receptors activated by pressure-induced application of 10 mM glycine in the vehicle-treated group. The top trace was generated from the high-pass filtered current and boxes indicate portions for the response subjected to variance analysis.

FIG. 3G shows the corresponding data for the EU1180-438-treated group.

FIG. 3H shows current variance plots of control (i.e., the vehicle-treated group).

FIG. 3I shows current variance plots of the EU1180-438-treated group.

FIG. 4A shows inhibition by 10 μM EU1180-438 of rat GluN1/human GluN3 (r1/h3A) receptors harboring pre-M1 mutations activated by 1 mM glycine in the presence of 0.5 μM CGP-78608, shown as mean±95% CI (error bars). The dashed lines show 95% CI of wild type. The “*” signs indicate non-overlapping confidence intervals.

FIG. 4B shows inhibition by 10 μM EU1180-438 of human GluN1^{FA,TL}/GluN3A (hl-FATL/h3A) receptors in the absence of CGP-78608, shown as mean±95% CI (error bars). The dashed lines show 95% CI of hl-FATL/h3A. The “*” signs indicate non-overlapping confidence intervals.

FIG. 5 shows dose-response curves for EU1180-481, EU1180-487, EU1180-488, and EU1180-489, in comparison with EU1180-438.

### DETAILED DESCRIPTION

The present disclosure describes negative allosteric modulators that selectively inhibits GluN3-containing NMDA receptors, such as those containing GluN3A or GluN3B.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

To the extent that chemical formula reported herein contain one or more unspecified chiral centers, the formulas are intended to encompass all stable stereoisomers, enantiomers, and diastereomers. It is also understood that formula encompass all tautomeric forms.

### I. Definitions

It must be noted that, as used in the specification, the singular forms “a,” “an,” and “the” include plural referents unless the context clearly dictates otherwise.

“Subject” refers to any animal, preferably a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

As used herein, “alkyl” means a noncyclic straight chain or branched chain, unsaturated or saturated hydrocarbon such as those containing from 1 to 25 carbon atoms. For example, a “C₈-C₁₈” refers to an alkyl containing 8 to 18 carbon atoms. Likewise, a “C₆-C₂₂” refers to an alkyl containing 6 to 22 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

As used herein, “heteroalkyl” refers to alkyl groups where one or more carbon atoms are replaced with a heteroatom, such as, O, N, or S. Similar to alkyl groups, heteroalkyl groups can be straight or branched, saturated or saturated. Optionally, the nitrogen and/or sulphur heteroatom(s) can be oxidized, and the nitrogen heteroatom(s) can be quaternized. Suitable heteroalkyl groups may contain 1 to 25 carbon atoms and 1 to 4 heteroatoms.

Non-aromatic, mono or polycyclic alkyls are referred to as “carbocycles” or “carbocyclyl” groups. Representative saturated carbocycles include cyclopropyl, cyclobutyl, cyclopentyl, cyclohexyl, and the like; while representative unsaturated carbocycles include cyclopentenyl, cyclohexenyl, and the like. In polycyclic carbocyclyl groups, the rings can be attached together in a pendant manner (i.e., two rings are connected by a single bond), in a spiro manner (i.e., two rings are connected through a defining single common atom), in a fused manner (i.e., two rings share two adjacent atoms; in other words, two rings share one covalent bond), in a bridged manner (i.e., two rings share three or more atoms, separating the two bridgehead atoms by a bridge containing at least one atom), or a combination thereof. The number of “members” of a carbocyclyl group refers to the total number of carbon atoms in the ring(s) of the carbocyclyl group.

“Heterocarbocycles” or “heterocarbocyclyl” groups are carbocycles which contain from 1 to 4 heteroatoms independently selected from nitrogen, oxygen, and sulphur, wherein the nitrogen and/or sulphur heteroatom(s) may be optionally oxidized, and the nitrogen heteroatom(s) may be optionally quaternized. Heterocarbocycles may be saturated or unsaturated (but not aromatic), monocyclic or polycyclic. Exemplary heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, and the like. The number of “members” of a heterocarbocyclyl group refers to the total number of carbon atoms and heteroatoms in the ring(s) of the heterocarbocyclyl group.

The term “aryl” refers to aromatic homocyclic (i.e., hydrocarbon) mono-, bi- or tricyclic ring-containing groups, preferably having 6 to 12 members, such as phenyl, naphthyl and biphenyl. Optionally, the aryl group is phenyl. In polycyclic aryl groups, the rings can be attached together in a pendant manner or can be fused. The number of “members” of an alkyl group refers to the total number of carbon atoms in the ring(s) of the alkyl group.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl (pyridinyl), quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent. The number of “members” of a heteroaryl group refers to the total number of carbon atoms and heteroatoms in the ring(s) of the heteroaryl group.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like. In polycyclic heterocyclyl groups, the rings can be attached together in a pendant manner (i.e., two rings are connected by a single bond), in a spiro manner (i.e., two rings are connected through a defining single common atom), in a fused manner (i.e., two rings share two adjacent atoms; in other words, two rings share one covalent bond), in a bridged manner (i.e., two rings share three or more atoms, separating the two bridgehead atoms by a bridge containing at least one atom), or a combination thereof. The number of “members” of a heterocyclyl group refers to the total number of carbon atoms and heteroatoms in the ring(s) of the heterocyclyl group.

“Alkoxy” or “alkyloxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, and t-butoxy.

“Alkylamino” refers an alkyl group as defined above with the indicated number of carbon atoms attached through an amino bridge. An example of an alkylamino is methylamino (i.e., —NH—CH₃).

“Alkylthio” refers to an alkyl group as defined above with the indicated number of carbon atoms attached through a sulfur bridge. An example of an alkylthio is methylthio (i.e., —S—CH₃).

The terms “cycloalkyl” and “cycloalkenyl” refer to mono-, bi-, or tri homocyclic ring groups of 3 to 15 carbon atoms which are, respectively, fully saturated and partially unsaturated (but not aromatic).

The terms “halogen” and “Hal” refer to fluorine, chlorine, bromine, and iodine.

The term “substituted” refers to a molecule wherein at least one hydrogen atom is replaced with a substituent. The molecule may be multiply substituted. In the case of an oxo substituent (“═O”), two hydrogen atoms are replaced. Example substituents within this context may include halogen, hydroxyl, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NR^{m}Rⁿ, —NR^{m}C(═O)Rⁿ, —NR^{m}C(═O)NRⁿR^{o}, —NR^{m}C(═O)ORⁿ, —NR^{m}SO₂Rⁿ, —C(═O)R^{m}, —C(═O)OR^{m}, —C(═O)NR^{m}Rⁿ, —OC(═O)NR^{m}Rⁿ, —OR^{m}, —SR^{m}, —SORⁿ, —S(═O)₂R^{m}, —OS(═O)₂R^{m}, and —S(═O)₂OR^{m}. R^{m}, Rⁿ, and R^{o} in this context may be the same or different, and independently hydrogen, halogen, hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, or heteroarylalkyl.

The term “optionally substituted,” as used herein, means that substitution is optional and therefore it is possible for the designated atom to be unsubstituted.

It is understood that any substitution is in accordance with permitted valence of the substituted atom and the substituent, and that the substitution results in a stable compound, i.e., a compound that does not spontaneously undergo transformation such as by rearrangement, cyclization, elimination, etc., at room temperature.

As used herein, the term “derivative” refers to a structurally similar compound that retains sufficient functional attributes of the identified analog. The derivative may be structurally similar because it is lacking one or more atoms, substituted, a salt, in different hydration/oxidation states, or because one or more atoms within the molecule are switched, such as, but not limited to, adding a hydroxyl group, replacing an oxygen atom with a sulfur atom, or replacing an amino group with a hydroxyl group, oxidizing a hydroxyl group to a carbonyl group, reducing a carbonyl group to a hydroxyl group, and reducing a carbon-to-carbon double bond to an alkyl group or oxidizing a carbon-to-carbon single bond to a double bond. A derivative optional has one or more, the same or different, substitutions. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) by Michael B. Smith or Domino Reactions in Organic Synthesis, Wiley (2006) by Lutz F. Tietze, which are hereby incorporated by reference.

### II. Compounds

Disclosed are negative allosteric modulators of GluN3-containing NMDA receptors, such as those containing GluN3A or GluN3B. These compounds are highly selective for GluN3 over GluN1 and/or GluN2.

The compounds can produce robust inhibition of glycine-activated current responses mediated by native GluN3-containing NMDA receptors in hippocampal CA1 pyramidal neurons.

They can function as non-competitive antagonists with activity that is independent of membrane potential (i.e., voltage-independent), glycine concentration, and extracellular pH.

To the extent that chemical formulas described herein contain one or more unspecified chiral centers, the formulas are intended to encompass all stable stereoisomers, enantiomers, and diastereomers. Such compounds can exist as a single enantiomer, a mixture of diastereomers, a racemic mixture, or combinations thereof. It is also understood that the chemical formulas encompass all tautomeric forms.

As used herein, “acyl” refers —C(═O)R^{A}, wherein R^{A} is an alkyl group, a heteroalkyl group, a carbocyclyl group, a heterocyclyl group, an aryl group, or a heteroaryl group.

As used herein, “sulfinyl” refers to —S(═O)R^{B}, wherein R^{B} is an alkyl group, a heteroalkyl group, a carbocyclyl group, a heterocyclyl group, an aryl group, or a heteroaryl group.

As used herein, “sulfonyl” refers to —S(═O)₂R^{C}, wherein R^{C} is an alkyl group, a heteroalkyl group, a carbocyclyl group, a heterocyclyl group, an aryl group, or a heteroaryl group.

As used herein, “pharmaceutically acceptable salt” refers to the modification of the original compound by making the acid or base salts thereof. Examples of pharmaceutically acceptable salts include, but are not limited to, mineral or organic acid salts of basic residues such as amines, as well as alkali or organic salts of acidic residues such as carboxylic acids or phosphorus acids. For original compounds containing a basic residue, the pharmaceutically acceptable salts can be prepared by treating the compounds with an appropriate amount of a non-toxic inorganic or organic acid; alternatively, the pharmaceutically acceptable salts can be formed in situ during preparation of the original compounds. Exemplary salts of the basic residue include salts with an inorganic acid selected from hydrochloric, hydrobromic, sulfuric, sulfamic, phosphoric, and nitric acids or with an organic acid selected from acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic acids. For original compounds containing an acidic residue, the pharmaceutically acceptable salts can be prepared by treating the compounds with an appropriate amount of a non-toxic base; alternatively, the pharmaceutically acceptable salts can be formed in situ during preparation of the original compounds. Exemplary salts of the acidic residue include salts with a base selected from ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, and histidine. Optionally, the pharmaceutically acceptable salts can be prepared by reacting the free acid or base form of the original compounds with a stoichiometric amount or more of the appropriate base or acid, respectively, in water, in an organic solvent, or in a mixture thereof. Lists of suitable pharmaceutically acceptable salts can be found in Remington's Pharmaceutical Sciences, 20th Ed., Lippincott Williams & Wilkins, Baltimore, M D, 2000; and Handbook of Pharmaceutical Salts: Properties, Selection, and Use, Stahl and Wermuth, Eds., Wiley-VCH, Weinheim, 2002.

Methods of making exemplary compounds are disclosed in the Examples. The methods are compatible with a wide variety of functional groups and compounds, and thus a wide variety of derivatives can be obtainable from the disclosed methods.

A. General Structure

Generally, the compounds have a structure of Formula I, an enantiomer or diastereomer thereof, or a pharmaceutically acceptable salt thereof,

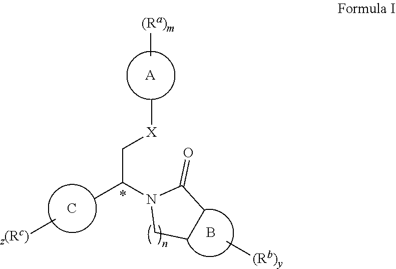

wherein rings A, B and C are individually and independently five- or six-membered aryl, heteroaryl, carbocycle, or heterocycle;
wherein X is O, S, or NH;
wherein n is 1, 2 or 3;
wherein m, y, and z are individually and independently 0, 1, 2, 3, or 4; and
wherein R^{a}, R^{b}, and R^{c}, in each occurrence, are individually and independently selected from hydrogen, deuterium, halogen, nitro, cyano, hydroxyl, trifluoromethoxy, trifluoromethyl, alkylsilyl (such as trimethylsilyl, methyl(methyl)(ethyl)silyl, triethylsilyl, triisopropylsilyl, methyl(methyl)(tert-butyl)silyl, methyl(methyl)(isobutyl)silyl), formyl, carboxyl, mercapto, sulfamoyl, alkyl (such as methyl, ethyl, isopropyl, tert-butyl), alkyloxy (such as methoxy, ethoxy), acyl (such as acetyl), acyloxy (such as acetoxy), amino, alkylamino (such as methylamino, ethylamino, dimethylamino, diethylamino, N-methyl-N-ethylamino), acylamino (acetylamino), carbamoyl, N-alkylcarbamoyl (such as N-methylcarbamoyl, N-ethylcarbamoyl, N,N-dimethylcarbamoyl, N,N-diethylcarbamoyl, N-methyl-N-ethylcarbamoyl), alkylthio (such as methylthio, ethylthio), alkylsulfinyl (such as methylsulfinyl, ethylsulfinyl), alkylsulfonyl (such as mesyl, ethylsulfonyl), alkyloxycarbonyl (such as methoxycarbonyl, ethoxycarbonyl), N-alkylsulfamoyl (such as N-methylsulfamoyl, N-ethylsulfamoyl, N,N-dimethylsulfamoyl, N,N-diethylsulfamoyl, N-methyl-N-ethylsulfamoyl), arylalkyl (such as benzyl), arylcarbonyl (such as benzoyl), heteroalkyl, carbocyclyl, heterocyclyl, aryl, and heteroaryl.

It is understood that ring B is fused with the rest of the ring structure containing the cyclic amide moiety.

Optionally, R^{a}, R^{b}, and R^{c}, in each occurrence, can be individually and independently substituted by one or more substituents. Exemplary substituents include halogen, hydroxyl, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NR^{m}Rⁿ, —NR^{m}C(═O)Rⁿ, —NR^{m}C(═O)NR^{m}R^{o}, —NR^{m}C(═O)ORⁿ, —NR^{m}SO₂Rⁿ, —C(═O)R^{m}, —C(═O)OR^{m}, —C(═O)NR^{m}Rⁿ, —OC(═O)NR^{m}Rⁿ, —OR^{m}, —SR^{m}, —SORⁿ, —S(═O)₂R^{m}, —OS(═O)₂R^{m}, and —S(═O)₂OR^{m}. R^{m}, Rⁿ, and R^{o} may be the same or different, and independently hydrogen, halogen, hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, or heteroarylalkyl.

In some embodiments, X is O. In some embodiments, X is S. In some embodiments, X is NH.

In some embodiments, rings A, B and C are individually and independently five- or six-membered aryl or heteroaryl. Optionally, ring A is phenyl or pyridinyl (such as 2-pyridinyl).

Optionally, ring B is phenyl. Optionally, ring C is phenyl.

In some embodiments, n is 1 or 2. In some embodiments, m is 1. In some embodiments, y is 0 or 1. In some embodiments, z is 0 or 1.

In some embodiments, R^{a}, R^{b}, and R^{c}, in each occurrence, are individually and independently selected from hydrogen, deuterium, halogen, nitro, cyano, hydroxyl, trifluoromethoxy, trifluoromethyl, trimethylsilyl, formyl, carboxyl, carbamoyl, mercapto, sulfamoyl, alkyl, alkoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, benzyl, benzoyl, carbocyclyl, aryl, and heterocyclyl. Optionally, R^{a}, R^{b}, and R^{c}, in each occurrence, can be individually and independently substituted by one or more substituents described above.

In some embodiments, R^{a}, R^{b}, and R^{c}, in each occurrence, are individually and independently selected from hydrogen, halogen, hydroxyl, alkyl (such as methyl), trifluoromethyl, trifluoromethoxy, and alkyloxy (such as methoxy, ethoxy, and isopropoxy). In some embodiments, R^{a} is alkyl (such as methyl), trifluoromethyl, trifluoromethoxy, or alkyloxy (such as methoxy, ethoxy, and isopropoxy). In some embodiments, R^{b} is alkyl (such as methyl), trifluoromethyl, trifluoromethoxy, or alkyloxy (such as methoxy, ethoxy, and isopropoxy). In some embodiments, R^{c} is halogen. In some embodiments, R^{a}, R^{b}, and R^{c}, in each occurrence, are individually and independently selected from the group consisting of hydrogen, halogen, hydroxyl, methyl, trifluoromethyl, trifluoromethoxy, methoxy, ethoxy, and isopropoxy.

In some embodiments, m is 1, and R^{a} is alkyl (such as methyl), trifluoromethyl, trifluoromethoxy, or alkyloxy (such as methoxy, ethoxy, and isopropoxy).

In some embodiments, y is 1, and R^{b} is alkyl (such as methyl), trifluoromethyl, trifluoromethoxy, or alkyloxy (such as methoxy, ethoxy, and isopropoxy).

In some embodiments, z is 1 or 2, and R^{c}, in each occurrence, is halogen.

The compounds of Formula I can be in either one of the following configurations:

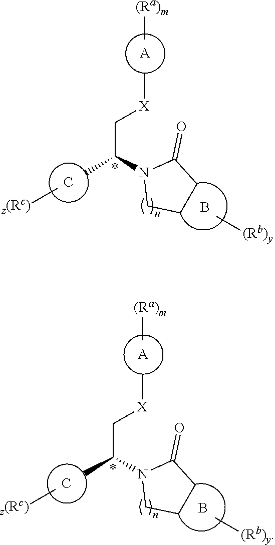

B. Exemplary Structures

Optionally, the compounds have a structure of Formula II, an enantiomer or diastereomer thereof, or a pharmaceutically acceptable salt thereof,

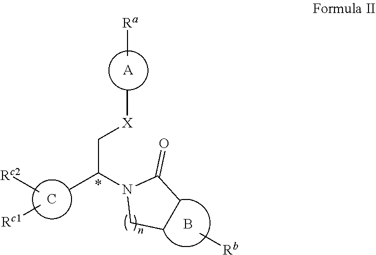

wherein rings A, B and C are individually and independently five- or six-membered aryl, heteroaryl, carbocycle, or heterocycle;
wherein X is O, S, or NH;
wherein n is 1, 2 or 3; and
wherein R^{a}, R^{b}, R^{c1}, and R^{c2}, in each occurrence, are individually and independently selected from hydrogen, deuterium, halogen, nitro, cyano, hydroxyl, trifluoromethoxy, trifluoromethyl, alkylsilyl (such as trimethylsilyl, methyl(methyl)(ethyl)silyl, triethylsilyl, triisopropylsilyl, methyl(methyl)(xtert-butyl)silyl, methyl(methyl)(isobutyl)silyl), formyl, carboxyl, mercapto, sulfamoyl, alkyl (such as methyl, ethyl, isopropyl, tert-butyl), alkyloxy (such as methoxy, ethoxy), acyl (such as acetyl), acyloxy (such as acetoxy), amino, alkylamino (such as methylamino, ethylamino, dimethylamino, diethylamino, N-methyl-N-ethylamino), acylamino (acetylamino), carbamoyl, N-alkylcarbamoyl (such as N-methylcarbamoyl, N-ethylcarbamoyl, N,N-dimethylcarbamoyl, N,N-diethylcarbamoyl, N-methyl-N-ethylcarbamoyl), alkylthio (such as methylthio, ethylthio), alkylsulfinyl (such as methylsulfinyl, ethylsulfinyl), alkylsulfonyl (such as mesyl, ethylsulfonyl), alkyloxycarbonyl (such as methoxycarbonyl, ethoxycarbonyl), N-alkylsulfamoyl (such as N-methylsulfamoyl, N-ethylsulfamoyl, N,N-dimethylsulfamoyl, N,N-diethylsulfamoyl, N-methyl-N-ethylsulfamoyl), arylalkyl (such as benzyl), arylcarbonyl (such as benzoyl), heteroalkyl, carbocyclyl, heterocyclyl, aryl, and heteroaryl.

Optionally, R^{a}, R^{b}, R^{c1}, and R^{c2}, in each occurrence, can be individually and independently substituted by one or more substituents, such as those described above.

In some embodiments, X is O. In some embodiments, X is S. In some embodiments, X is NH.

In some embodiments, rings A, B and C are individually and independently five- or six-membered aryl or heteroaryl. Optionally, ring A is phenyl or pyridinyl (such as 2-pyridinyl). Optionally, ring B is phenyl. Optionally, ring C is phenyl.

In some embodiments, n is 1 or 2.

In some embodiments, rings A, B and C are individually and independently five- or six-membered aryl or heteroaryl, X is O, and n is 1 or 2.

In some embodiments, R^{a}, R^{b}, R^{c1}, and R^{c2}, in each occurrence, are individually and independently selected from hydrogen, deuterium, halogen, nitro, cyano, hydroxyl, trifluoromethoxy, trifluoromethyl, trimethylsilyl, formyl, carboxyl, carbamoyl, mercapto, sulfamoyl, alkyl, alkoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, benzyl, benzoyl, carbocyclyl, aryl, and heterocyclyl. Optionally, R^{a}, R^{b}, R^{c1}, and R^{c2}, in each occurrence, can be individually and independently substituted by one or more substituents described above.

In some embodiments, R^{a}, R^{b}, R^{c1}, and R^{c2}, in each occurrence, are individually and independently selected from hydrogen, halogen, hydroxyl, alkyl (such as methyl), trifluoromethyl, trifluoromethoxy, and alkyloxy (such as methoxy, ethoxy, and isopropoxy). In some embodiments, R^{a} is alkyl (such as methyl), trifluoromethyl, trifluoromethoxy, or alkyloxy (such as methoxy, ethoxy, and isopropoxy). In some embodiments, R^{b} is alkyl (such as methyl), trifluoromethyl, trifluoromethoxy, or alkyloxy (such as methoxy, ethoxy, and isopropoxy). In some embodiments, R^{c1} and R^{c2} are halogen. In some embodiments, R^{a}, R^{b}, R^{c1}, and R^{c2}, in each occurrence, are individually and independently selected from the group consisting of hydrogen, halogen, hydroxyl, methyl, trifluoromethyl, trifluoromethoxy, methoxy, ethoxy, and isopropoxy.

The compounds of Formula II can be in either one of the following configurations:

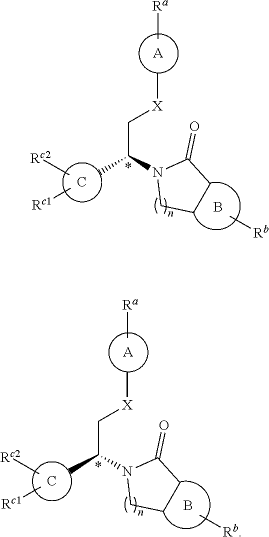

Optionally, the compounds have a structure of Formula IIA, an enantiomer or diastereomer thereof, or a pharmaceutically acceptable salt thereof,

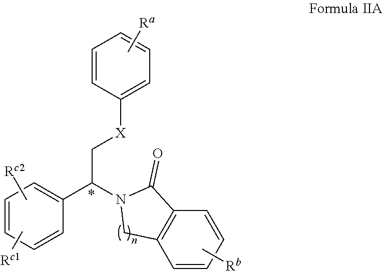

wherein X, n, R^{a}, R^{b}, R^{c1}, and R^{c2} are the same as those described in Formula II.

In some embodiments, R^{a}, R^{b}, R^{c1}, and R^{c2}, in each occurrence, are individually and independently selected from hydrogen, halogen, hydroxyl, alkyl (such as methyl), trifluoromethyl, trifluoromethoxy, and alkyloxy (such as methoxy, ethoxy, and isopropoxy). In some embodiments, R^{a} is alkyl (such as methyl), trifluoromethyl, trifluoromethoxy, or alkyloxy (such as methoxy, ethoxy, and isopropoxy). In some embodiments, R^{b} is alkyl (such as methyl), trifluoromethyl, trifluoromethoxy, or alkyloxy (such as methoxy, ethoxy, and isopropoxy). In some embodiments, R^{c1} and R^{c2} are halogen. In some embodiments, R^{a}, R^{b}, R^{c1}, and R^{c2}, in each occurrence, are individually and independently selected from the group consisting of hydrogen, halogen, hydroxyl, methyl, trifluoromethyl, trifluoromethoxy, methoxy, ethoxy, and isopropoxy.

In some embodiments, R^{a} is methoxy, optionally at the 4 position. In some embodiments, R^{b} is hydrogen.

The compounds of Formula IIA can be in either one of the following configurations:

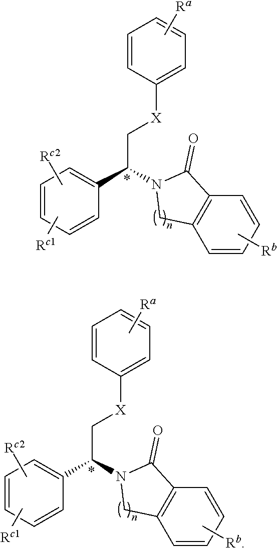

Optionally, the compounds have a structure of Formula IIB, an enantiomer or diastereomer thereof, or a pharmaceutically acceptable salt thereof,

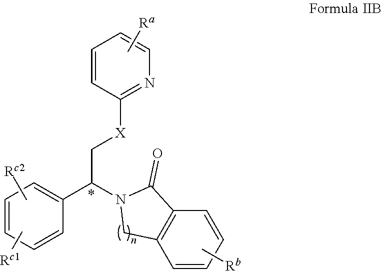

wherein X, n, R^{a}, R^{b}, R^{c1}, and R^{c2} are the same as those described in Formula II.

In some embodiments, R^{a}, R^{b}, R^{c1}, and R^{c2}, in each occurrence, are individually and independently selected from hydrogen, halogen, hydroxyl, alkyl (such as methyl), trifluoromethyl, trifluoromethoxy, and alkyloxy (such as methoxy, ethoxy, and isopropoxy). In some embodiments, R^{a} is alkyl (such as methyl), trifluoromethyl, trifluoromethoxy, or alkyloxy (such as methoxy, ethoxy, and isopropoxy). In some embodiments, R^{b} is alkyl (such as methyl), trifluoromethyl, trifluoromethoxy, or alkyloxy (such as methoxy, ethoxy, and isopropoxy). In some embodiments, R^{c1} and R^{c2} are halogen. In some embodiments, R^{a}, R^{b}, R^{c1}, and R^{c2}, in each occurrence, are individually and independently selected from the group consisting of hydrogen, halogen, hydroxyl, methyl, trifluoromethyl, trifluoromethoxy, methoxy, ethoxy, and isopropoxy.

In some embodiments, R^{a} is methyl, optionally at the 3 position. In some embodiments, R^{b} is hydrogen.

The compounds of Formula IIB can be in either one of the following configurations:

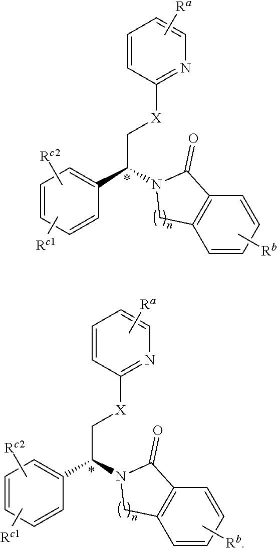

C. Exemplary Compounds

Exemplary compounds are described in the Examples.

In some embodiments, the compounds are selected from:
2-(2-(4-methoxyphenoxy)-1-(4-methoxyphenyl)ethyl)isoindolin-1-one,
2-(1-(4-isopropoxyphenyl)-2-(4-methoxyphenoxy)ethyl)isoindolin-1-one,
2-(2-(4-methoxyphenoxy)-1-phenyl-ethyl)isoindolin-1-one,
2-(2-(4-methoxyphenoxy)-1-phenylethyl)-3,4-dihydroisoquinolin-1(2H)-one,
2-(2-(4-methoxyphenoxy)-1-(4-methoxyphenyl)ethyl)-3,4-dihydroisoquinolin-1(2H)-one,
2-(2-(4-ethoxyphenoxy)-1-(4-isopropoxyphenyl)ethyl)isoindolin-1-one,
2-(1-(4-isopropoxyphenyl)-2-((6-methylpyridin-2-yl)oxy)ethyl)isoindolin-1-one,
6-fluoro-2-(1-(4-isopropoxyphenyl)-2-((6-methylpyridin-2-yl)oxy)ethyl)isoindolin-1-one,
6-chloro-2-(2-(4-methoxyphenoxy)-1-(4-methoxyphenyl)ethyl)isoindolin-1-one,
6-bromo-2-(2-(4-methoxyphenoxy)-1-(4-methoxyphenyl)ethyl)isoindolin-1-one,
5-fluoro-2-(2-(4-methoxyphenoxy)-1-(4-methoxyphenyl)ethyl)isoindolin-1-one,
6-fluoro-2-(2-(4-methoxyphenoxy)-1-(4-methoxyphenyl)ethyl)isoindolin-1-one,
2-(1-(2-fluoro-4-methoxyphenyl)-2-(4-methoxyphenoxy)ethyl)isoindolin-1-one,
6-chloro-2-(1-(2-fluoro-4-methoxyphenyl)-2-(4-methoxyphenoxy)ethyl)isoindolin-1-one,
6-fluoro-2-(1-(2-fluoro-4-methoxyphenyl)-2-(4-methoxyphenoxy)ethyl)isoindolin-1-one,
6-fluoro-2-(1-(4-isopropoxyphenyl)-2-(4-methoxyphenoxy)ethyl)isoindolin-1-one,
6-chloro-2-(2-(4-ethoxyphenoxy)-1-(4-isopropoxyphenyl)ethyl)isoindolin-1-one,
6-fluoro-2-(2-(4-ethoxyphenoxy)-1-(4-isopropoxyphenyl)ethyl)isoindolin-1-one,
6-bromo-2-(1-(4-methoxyphenyl)-2-((6-methylpyridin-2-yl)oxy)ethyl)isoindolin-1-one,
6-fluoro-2-(1-(4-methoxyphenyl)-2-((6-methylpyridin-2-yl)oxy)ethyl)isoindolin-1-one,
6-chloro-2-(1-(2-fluoro-4-methoxyphenyl)-2-((6-methylpyridin-2-yl)oxy)ethyl)isoindolin-1-one,
5-bromo-2-(1-(2-fluoro-4-methoxyphenyl)-2-((6-methylpyridin-2-yl)oxy)ethyl)isoindolin-1-on,
6-bromo-2-(1-(2-fluoro-4-methoxyphenyl)-2-((6-methylpyridin-2-yl)oxy)ethyl)isoindolin-1-one,
6-chloro-2-(1-(2,6-difluoro-4-methoxyphenyl)-2-((6-methylpyridin-2-yl)oxy)ethyl)isoindolin-1-one, and
2-(1-(2,6-difluoro-4-methoxyphenyl)-2-((6-methylpyridin-2-yl)oxy)ethyl)-6-fluoroisoindolin-1-one.

In some embodiments, the compounds are selected from:
2-(2-(4-methoxyphenoxy)-1-(4-methoxyphenyl)ethyl)isoindolin-1-one,
2-(1-(4-isopropoxyphenyl)-2-(4-methoxyphenoxy)ethyl)isoindolin-1-one,
2-(2-(4-methoxyphenoxy)-1-phenyl-ethyl)isoindolin-1-one,
2-(2-(4-methoxyphenoxy)-1-phenylethyl)-3,4-dihydroisoquinolin-1(2H)-one,
2-(2-(4-methoxyphenoxy)-1-(4-methoxyphenyl)ethyl)-3,4-dihydroisoquinolin-1(2H)-one,
2-(1-(4-isopropoxyphenyl)-2-((6-methylpyridin-2-yl)oxy)ethyl)isoindolin-1-one,
6-fluoro-2-(1-(4-isopropoxyphenyl)-2-((6-methylpyridin-2-yl)oxy)ethyl)isoindolin-1-one,
6-chloro-2-(2-(4-methoxyphenoxy)-1-(4-methoxyphenyl)ethyl)isoindolin-1-one,
5-fluoro-2-(2-(4-methoxyphenoxy)-1-(4-methoxyphenyl)ethyl)isoindolin-1-one,
6-fluoro-2-(2-(4-methoxyphenoxy)-1-(4-methoxyphenyl)ethyl)isoindolin-1-one,
2-(1-(2-fluoro-4-methoxyphenyl)-2-(4-methoxyphenoxy)ethyl)isoindolin-1-one,
6-chloro-2-(1-(2-fluoro-4-methoxyphenyl)-2-(4-methoxyphenoxy)ethyl)isoindolin-1-one,
6-fluoro-2-(1-(4-isopropoxyphenyl)-2-(4-methoxyphenoxy)ethyl)isoindolin-1-one,
6-fluoro-2-(2-(4-ethoxyphenoxy)-1-(4-isopropoxyphenyl)ethyl)isoindolin-1-one,
6-fluoro-2-(1-(4-methoxyphenyl)-2-((6-methylpyridin-2-yl)oxy)ethyl)isoindolin-1-one,
6-chloro-2-(1-(2,6-difluoro-4-methoxyphenyl)-2-((6-methylpyridin-2-yl)oxy)ethyl)isoindolin-1-one, and
2-(1-(2,6-difluoro-4-methoxyphenyl)-2-((6-methylpyridin-2-yl)oxy)ethyl)-6-fluoroisoindolin-1-one.

D. Additional Structures and Compounds

In accordance with an embodiment of the present disclosure there is provided a compound of Formula III or a salt thereof,

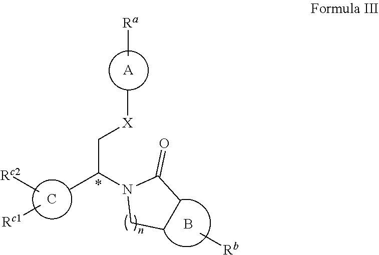

wherein rings A, B and C are individually and independently selected from a five- or six-membered aryl, heteroaryl, carbocycle, and heterocycle;
X is O, S or NH;
R^{a}, R^{b}, R^{c1} and R^{c2} are individually and independently selected from hydrogen, deuterium, halogen, nitro, cyano, hydroxyl, trifluoromethoxy, trifluoromethyl, trimethylsilyl, formyl, carboxyl, carbamoyl, mercapto, sulfamoyl, alkyl, alkoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, benzyl, benzoyl, carbocyclyl, aryl, and heterocyclyl; and
n is 1, 2 or 3.

In certain embodiments, the disclosure contemplates derivatives of the compound, such as those containing one or more, the same or different, substituents. For example, R^{a}, R^{b}, R^{c1}, and R^{c2}, in each occurrence, can be individually and independently substituted by one or more substituents, such as those described above.

Further features of this embodiment provide for R^{a}, R^{b}, R^{c1} and R^{c2} to be individually and independently selected from hydrogen, halogen, hydroxyl, methyl, trifluoromethyl, trifluoromethoxy, methoxy, ethoxy, and isopropoxy. Yet further features provide for R^{a}, R^{c1} and R^{c2} to be individually and independently selected from hydrogen, halogen, hydroxyl, methyl, trifluoromethyl, trifluoromethoxy, methoxy, ethoxy, and isopropoxy and for R^{b} to be a hydrogen.

The compounds of Formula III can be in either one of the following configurations:

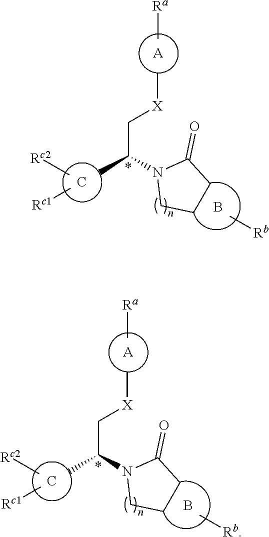

In accordance with another embodiment of the present disclosure there is provided a compound of Formula IIIA or a salt thereof,

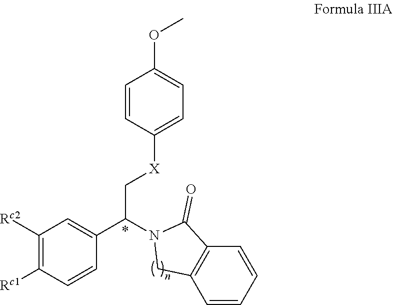

wherein X is O, S, or NH;
R^{c1} and R^{c2} are individually and independently selected from hydrogen, deuterium, halogen, nitro, cyano, hydroxyl, trifluoromethoxy, trifluoromethyl, trimethylsilyl, formyl, carboxyl, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, benzyl, benzoyl, carbocyclyl, aryl, and heterocyclyl; and
n is 1, 2 or 3.

The compounds of Formula IIIA can be in either one of the following configurations:

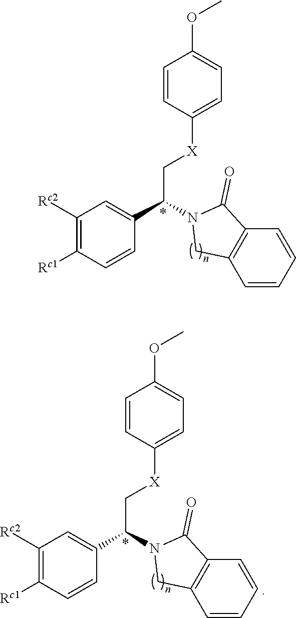

Exemplary compounds of Formulas III and IIIA include, but are not limited to:

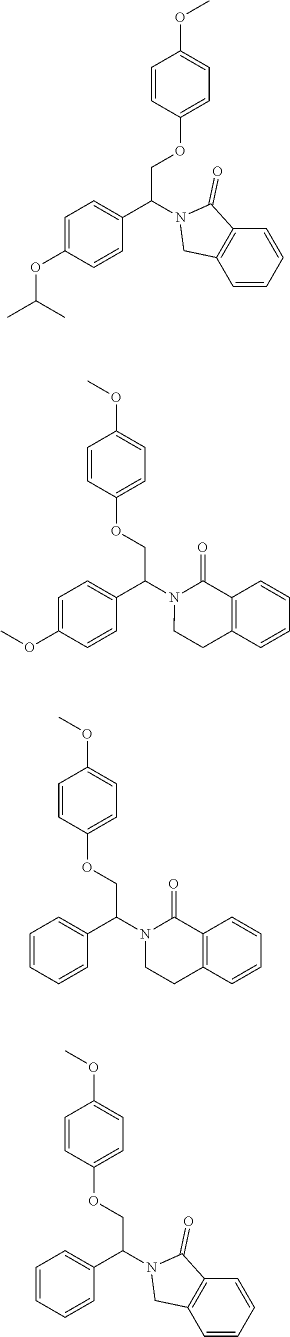 [SMILES: COc1ccc(OCC(c2ccc(OC(C)C)cc2)N2Cc3ccccc3C2=O)cc1 ; COc1ccc(OCC(c2ccc(OC)cc2)N2CCc3ccccc3C2=O)cc1 ; COc1ccc(OCC(c2ccccc2)N2CCc3ccccc3C2=O)cc1 ; COc1ccc(OCC(c2ccccc2)N2Cc3ccccc3C2=O)cc1]

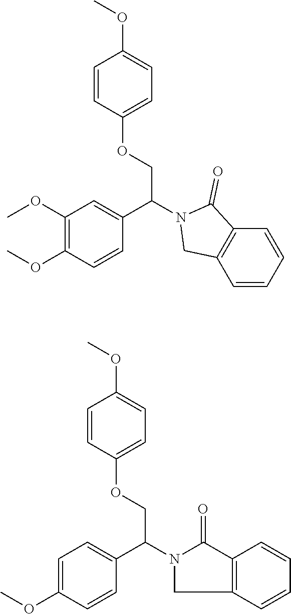 [SMILES: COc1ccc(OCC(c2ccc(OC)c(OC)c2)N2Cc3ccccc3C2=O)cc1 ; COc1ccc(OCC(c2ccc(OC)cc2)N2Cc3ccccc3C2=O)cc1]

### III. Compositions

The disclosed compounds may be present in a mixture of stereoisomers. In some embodiments, the compounds in the mixture of stereoisomers may be in greater than 60%, 70%, 80%, 90%, 95%, or 98% diastereomeric or enantiomeric excess. In some embodiments, the compounds in the mixture of stereoisomers may be in greater than 95% diastereomeric or enantiomeric excess.

Disclosed are compositions containing the afore-mentioned mixture of stereoisomers. In some embodiments, the compositions contain a compound disclosed herein, wherein the compound is in greater than 60%, 70%, 80%, 90%, 95%, or 98% enantiomeric excess with respect to the stereocenter labeled by the “*” sign in Formulas I, II, IIA, IIB, III, and IIIA.

For example, the compositions contain a compound disclosed herein, wherein the compound is in greater than 95% enantiomeric excess for the configuration depicted below:

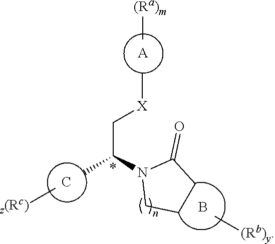

Another example, the compositions contain a compound disclosed herein, wherein the compound is in greater than 95% enantiomeric excess for the configuration depicted below:

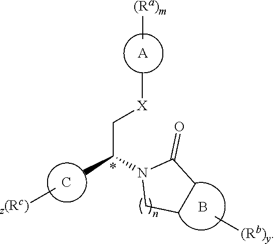

The disclosed compounds may be present in a mixture of the salt form and the non-salt form. In some embodiments, more than 50%, 60%, 70%, 80%, 90%, 95%, or 98% of the compound in the mixture may be in the salt form, calculated as the ratio of the weight of the salt form to the total weight of the salt form and the non-salt form. In some embodiments, more than 90% of the compound in the mixture may be in the salt form.

### IV. Formulations

Disclosed are pharmaceutical formulations containing a compound or composition described above. Generally, the pharmaceutical formulations also contain a pharmaceutically acceptable excipient. The pharmaceutical formulations may also include one or more further active agents or may be administered in combination with one or more such active agents.

The pharmaceutical formulations can be in the form of tablet, capsule, pill, caplets, cream, gel, granule, solution (such as aqueous solution, e.g., saline or buffered saline), emulsion, suspension, nanoparticle formulation, etc. In some embodiments, the pharmaceutical formulations are oral formulations. In some embodiments, the pharmaceutical formulations are intravenous formulations. In some embodiments, the pharmaceutical formulations are topical formulations.

The pharmaceutical formulations may be prepared in a manner known per se, which usually involves mixing a compound or composition according to the disclosure with the pharmaceutically acceptable excipient, and, if desired, in combination with other pharmaceutical active agent(s), when necessary under aseptic conditions.

As used herein, “emulsion” refers to a composition containing a mixture of non-miscible components homogenously blended together. In some forms, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion.

As used herein, “biocompatible” refers to materials that are neither themselves toxic to the host (e.g., a non-human animal or human), nor degrade (if the material degrades) at a rate that produces monomeric or oligomeric subunits or other byproducts at toxic concentrations in the host.

As used herein, “biodegradable” refers to materials degrades or breaks down into its component subunits, or digestion, e.g., by a biochemical process, of the material into smaller (e.g., non-polymeric) subunits.

As used herein, “enteric polymers” refer to polymers that become soluble in the higher pH environment of the lower gastrointestinal tract or slowly erode as the dosage form passes through the gastrointestinal tract.

As used herein, “enzymatically degradable polymers” refer to polymers that are degraded by bacterial enzymes present in the lower gastrointestinal tract, particularly in the colon.

As used herein, “pharmaceutically acceptable” refers to compounds, materials, compositions, and/or formulations which are, within the scope of sound medical judgment, suitable for use in contact with the tissues of human beings and animals without excessive toxicity, irritation, allergic response, or other problems or complications that commensurate with a reasonable benefit/risk ratio, in accordance with the guidelines of agencies such as the Food and Drug Administration (FDA).

As used herein, “nanoparticle” generally refers to particles having a diameter from about 1 nm to 1000 nm, preferably from about 10 nm to 1000 nm, more preferably from about 100 nm to 1000 nm, most preferably from about 250 nm to 1000 nm. In some embodiments, “nanoparticles” can also refer to “microparticles,” which are particles having a diameter from about 1 micron to about 100 microns, preferably from about 1 to about 50 microns, more preferably from about 1 to about 30 microns, most preferably from about 1 micron to about 10 microns. In some embodiments, the nanoparticles can be a mixture of nanoparticles, as defined above, and microparticles, as defined above.

As used herein, the term “surfactant” refers to any agent which preferentially absorbs to an interface between two immiscible phases, such as the interface between water and an organic polymer solution, a water/air interface, or organic solvent/air interface. Surfactants generally possess a hydrophilic moiety and a lipophilic moiety.

As used herein, “gel” is a semisolid system containing a dispersion of the active agent, i.e., the compound, in a liquid vehicle that is rendered semisolid by the action of a thickening agent or polymeric material dissolved or suspended in the liquid vehicle. The liquid vehicle may include a lipophilic component, an aqueous component or both.

As used herein, “hydrogel” refers to a swollen, water-containing network of finely-dispersed polymer chains that are water-insoluble, where the polymeric molecules are in the external or dispersion phase and water (or an aqueous solution) forms the internal or dispersed phase. The chains can be chemically cross-linked (chemical gels) or physically cross-linked (physical gels). Chemical gels possess polymer chains that are connected through covalent bonds, whereas physical gels have polymer chains linked by non-covalent bonds or cohesion forces, such as van der Waals interactions, ionic interaction, hydrogen bonding, or hydrophobic interaction.

As used herein, drug-containing “beads” refer to beads made with drug and one or more excipients. Drug-containing beads can be produced by applying drug to an inert support, e.g., inert sugar beads coated with drug or by creating a “core” comprising both drug and the one or more excipients. As is also known, drug-containing “granules” and “particles” comprise drug particles that may or may not include one or more additional excipients. Typically, granules and particles do not contain an inert support. Granules generally comprise drug particles and require further processing. Generally, particles are smaller than granules, and are not further processed. Although beads, granules and particles may be formulated to provide immediate release, beads and granules are generally employed to provide delayed release.

A. Physical Forms and Unit Dosages

Depending upon the manner of introduction, the compounds or compositions described herein may be formulated in a variety of ways. The pharmaceutical formulations can be prepared in various forms, such as granules, tablets, capsules, pills, caplets, suppositories, powders, controlled release formulations, nanoparticle formulations, solutions (such as aqueous solutions, e.g., saline, buffered saline), suspensions, emulsions, creams, gels, ointments, salves, lotions, aerosols, and the like.

In some embodiments, the pharmaceutical formulations are in solid dosage forms suitable for simple, and preferably oral, administration of precise dosages. Solid dosage forms for oral administration include, but are not limited to, tablets, soft or hard gelatin or non-gelatin capsules, and caplets. However, liquid dosage forms, such as solutions, syrups, suspensions (including nano- or microsuspensions), shakes, emulsions, etc. can also be utilized. Intravenous formulations are usually in liquid dosage forms, including solutions, emulsions, and suspensions. Suitable topical formulations include, but are not limited to, lotions, ointments, creams, and gels. In some embodiments, the topical formulations are in the form of gels or creams.

In some embodiments, the pharmaceutical formulations are in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages contain between 1 and 1000 mg, and usually between 5 and 500 mg, of at least one compound from the disclosure, e.g., about 10, 25, 50, 100, 200, 300 or 400 mg per unit dosage.

The concentration of the compound to the pharmaceutically acceptable excipient may vary from about 0.5 to about 100 wt %. For oral use, the pharmaceutical formulations generally contain from about 5 to about 100 wt % of the compound. For other uses, the pharmaceutical formulations generally have from about 0.5 to about 50 wt % of the compound.

B. Pharmaceutically Acceptable Excipients

As used herein, “excipient” refers to all components present in the pharmaceutical formulations other than the active ingredient(s). Pharmaceutically acceptable excipients are composed of materials that are considered safe and may be administered to an individual without causing undesirable biological side effects or unwanted interactions. For example, the pharmaceutically acceptable excipients can be compounds or materials recognized by the FDA as “generally recognized as safe” or “GRAS”.

Generally, excipients include, but are not limited to, diluents (fillers), binders, lubricants, disintegrants, pH modifying or buffering agents, preservatives, antioxidants, solubility enhancers, wetting or emulsifying agents, plasticizers, colorants (such as pigments and dyes), stabilizers, glidants, solvent or dispersion medium, surfactants, pore formers, and coating or matrix materials.

In some embodiments, drug-containing tablets, beads, granules or particles contain one or more of the following excipients: diluents, binders, lubricants, disintegrants, pigments, stabilizers, and surfactants. If desired, the tablets, beads, granules, or particles may also contain minor amount of nontoxic auxiliary substances such as wetting or emulsifying agents, dyes, pH buffering agents, or preservatives.

Examples coating or matrix materials include, but are not limited to, cellulose polymers (such as methylcellulose, ethyl cellulose, cellulose acetate, cellulose acetate phthalate, hydroxypropyl cellulose, hydroxyethyl cellulose, hydroxymethyl cellulose, hydroxypropyl methylcellulose, hydroxypropyl methylcellulose phthalate, hydroxypropyl methylcellulose acetate succinate, cellulose acetate trimellitate, and carboxymethylcellulose sodium), vinyl polymers and copolymers (such as polyvinyl pyrrolidone, polyvinyl acetate, polyvinyl acetate phthalate, vinyl acetate-crotonic acid copolymer, and ethylene-vinyl acetate copolymer), acrylic acid polymers and copolymers (such as those formed from acrylic acid, methacrylic acid, methyl acrylate, ethyl acrylate, methyl methacrylate and/or ethyl methacrylate, and other methacrylic resins that are commercially available under the tradename EUDRAGIT®), enzymatically degradable polymers (such as azo polymers, pectin, chitosan, amylose and guar gum), zein, shellac, and polysaccharides. In some embodiments, the coating or matrix materials may contain conventional excipients such as plasticizers, colorants, glidants, stabilizers, pore formers, and surfactants.

In some embodiments, the coating or matrix materials are pH-sensitive or pH-responsive polymers, such as the enteric polymers commercially available under the tradename EUDRAGIT®. For example, EUDRAGIT® L30D-55 and L100-55 are soluble at pH 5.5 and above; EUDRAGIT® L100 is soluble at pH 6.0 and above; EUDRAGIT® S is soluble at pH 7.0 and above, as a result of a higher degree of esterification.

In some embodiments, the coating or matrix materials are water-insoluble polymers having different degrees of permeability and expandability, such as EUDRAGIT® NE, RL, and RS.

Depending on the coating or matrix materials, the decomposition/degradation or structural change of the pharmaceutical formulations may occur at different locations of the gastrointestinal tract. In some embodiments, the coating or matrix materials are selected such that the pharmaceutical formulations can survive exposure to gastric acid and release the compound in the intestines after oral administration.

Diluents, also referred to as “fillers,” can increase the bulk of a solid dosage form so that a practical size is provided for compression of tablets or formation of beads, granules, or particles. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate, powdered sugar, and combinations thereof.

Binders are used to impart cohesive qualities to a solid dosage formulation, and thus ensure that a tablet, bead, granule, or particle remains intact after the formation of the dosage forms. Suitable binder materials include, but are not limited to, starch, pregelatinized starch, gelatin, sugars (such as sucrose, glucose, dextrose, lactose and sorbitol), polyethylene glycol, waxes, natural and synthetic gums (such as acacia, tragacanth, and sodium alginate), cellulose (such as hydroxypropylmethylcellulose, hydroxypropylcellulose, and ethylcellulose), veegum, and synthetic polymers (such as acrylic acid and methacrylic acid copolymers, methacrylic acid copolymers, methyl methacrylate copolymers, aminoalkyl methacrylate copolymers, polyacrylic acid, polymethacrylic acid, and polyvinylpyrrolidone), and combinations thereof.

Lubricants are used to facilitate tablet manufacture. Suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, gums or cross-linked polymers, such as cross-linked polyvinylpyrrolidone (e.g., POLYPLASDONE® XL from GAF Chemical Corp.).

Plasticizers are normally present to produce or promote plasticity and flexibility and to reduce brittleness. Examples of plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil, and acetylated monoglycerides.

Stabilizers are used to inhibit or retard decomposition reactions of the active agents in the formulations or stabilize particles in a dispersion. For example, when the decomposition reactions involve an oxidation reaction of an active agent in the formulations, the stabilizer can be an antioxidant or a reducing agent. Stabilizers also include nonionic emulsifiers such as sorbitan esters, polysorbates, and polyvinylpyrrolidone.

Glidants are used to reduce sticking effects during film formation and drying. Exemplary glidant include, but are not limited to talc, magnesium stearate, and glycerol monostearates.

Pigments such as titanium dioxide may also be used.

Preservatives can inhibit the deterioration and/or decomposition of a pharmaceutical formulation. Deterioration or decomposition can be brought about by any of microbial growth, fungal growth, and undesirable chemical or physical changes. Suitable preservatives include benzoate salts (e.g., sodium benzoate), ascorbic acid, methyl hydroxybenzoate, ethyl p-hydroxybenzoate, n-propyl p-hydroxybenzoate, n-butyl p-hydroxybenzoate, potassium sorbate, sorbic acid, proprionate salts (e.g., sodium propionate), chlorobutanol, benzyl alcohol, and combinations thereof.

Surfactants may be anionic, cationic, amphoteric or nonionic surface-active agents. Exemplary anionic surfactants include, but are not limited to, those containing carboxylate, sulfonate and sulfate ions. Examples of anionic surfactants include sodium, potassium, ammonium of long chain (e.g., 13-21) alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, polyxamers (such as poloxamer 401), stearoyl monoisopropanolamide, and polyoxyethylene hydrogenated tallow amide. Examples of amphoteric surfactants include, but are not limited to, sodium N-dodecyl-p-alanine, sodium N-lauryl-p-iminodipropionate, myristoamphoacetate, lauryl betaine and lauryl sulfobetaine.

Pharmaceutical formulations in liquid forms typically contain a solvent or dispersion medium such as water, aqueous solution (such as saline and buffered saline), ethanol, polyol (such as glycerol, propylene glycol, and liquid polyethylene glycol), oil (such as vegetable oil, e.g., peanut oil, corn oil, sesame oil, etc.), and combinations thereof. Preferably, the pharmaceutical formulations in liquid forms are aqueous formulations. Suitable solvent or dispersion medium for intravenous formulations include, but are not limited to, water, saline, buffered saline (such as phosphate buffered saline), and Ringer's solution.

C. Pharmaceutical Acceptable Carriers

In some embodiments, the pharmaceutical formulations are prepared using a pharmaceutically acceptable carrier, which encapsulates, embeds, entraps, dissolves, disperses, absorbs, or bind to a compound or composition disclosed herein. The pharmaceutical acceptable carrier is composed of materials that are considered safe and can be administered to a subject without causing undesirable biological side effects or unwanted interactions. Preferably, the pharmaceutically acceptable carrier does not interfere with the effectiveness of the compound or composition in performing its function. The pharmaceutically acceptable carrier can be formed of biodegradable materials, non-biodegradable materials, or combinations thereof. The pharmaceutical acceptable excipient described above may be partially or entirely present in the pharmaceutical acceptable carrier.

In some embodiments, the pharmaceutical acceptable carrier is a controlled-release carrier, such as delayed-release carriers, sustained-release (extended-release) carriers, and pulsatile-release carriers.

In some embodiments, the pharmaceutical acceptable carrier is pH-sensitive or pH-responsive. In some forms, the pharmaceutical acceptable carrier can decompose or degrade in a certain pH range. In some forms, the pharmaceutical acceptable carrier can experience a structural change when experiencing a change in the pH.

Exemplary pharmaceutical acceptable carriers include, but are not limited to, nanoparticles, liposomes, hydrogels, polymer matrices, and solvent systems.

In some embodiments, the pharmaceutical acceptable carrier is nanoparticles. In some forms, the compound is embedded in the matrix formed by materials of the nanoparticles.

The nanoparticles can be biodegradable, and preferably are capable of biodegrading at a controlled rate for delivery of the compound. The nanoparticles can be made of a variety of materials. Both inorganic and organic materials can be used. Both polymeric and non-polymeric materials can be used.

Preferably, the nanoparticles are polymeric nanoparticles formed of one or more biocompatible polymers, copolymers, or blends thereof. In some forms, the biocompatible polymers are biodegradable. In some forms, the biocompatible polymers are non-biodegradable. In some forms, the nanoparticles are formed of a mixture of biodegradable and non-biodegradable polymers. The polymers may be tailored to optimize different characteristics of the nanoparticles including: (i) interactions between the compound and the polymer to provide stabilization of the compound and retention of activity upon delivery; (ii) rate of polymer degradation and, thereby, rate of release; (iii) surface characteristics and targeting capabilities via chemical modification; and (iv) particle porosity.

Exemplary polymers include, but are not limited to, polymers prepared from lactones such as poly(caprolactone) (PCL), polyhydroxy acids and copolymers thereof such as poly(lactic acid) (PLA), poly(glycolic acid) (PGA), poly(lactic acid-co-glycolic acid) (PLGA), and blends thereof, polyalkyl cyanoacralate, polyurethanes, polyamino acids such as poly-L-lysine (PLL), poly(valeric acid), and poly-L-glutamic acid, hydroxypropyl methacrylate (HPMA), polyanhydrides, polyorthoesters, poly(ester amides), polyamides, poly(ester ethers), polycarbonates, ethylene vinyl acetate polymer (EVA), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), celluloses including derivatized celluloses such as alkyl celluloses, hydroxyalkyl celluloses, cellulose ethers, cellulose esters, nitro celluloses, hydroxypropylcellulose, and carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), and poly(octadecyl acrylate) (jointly referred to herein as “polyacrylic acids”), polydioxanone and its copolymers, polyhydroxyalkanoates, polypropylene fumarate, polyoxymethylene, poloxamers, poly(butyric acid), trimethylene carbonate, polyphosphazenes, polysaccharides, peptides or proteins, and copolymers or blends thereof.

Preferably, the polymer is an FDA approved biodegradable polymer such as polyhydroxy acids (e.g., PLA, PLGA, PGA), polyanhydride, polyhydroxyalkanoate such as poly(3-butyrate) or poly(4-butyrate), and copolymer or blends thereof.

Materials other than polymers may be used to form the nanoparticles. Suitable materials include excipients such as surfactants.

The use of surfactants in the nanoparticles may improve surface properties by, for example, reducing particle-particle interactions, and render the surface of the particles less adhesive. Both naturally occurring surfactants and synthetic surfactants can be incorporated into the nanoparticles. Exemplary surfactants include, but are not limited to, phosphoglycerides such as phosphatidylcholines (e.g., L-α-phosphatidylcholine dipalmitoyl), diphosphatidyl glycerol, hexadecanol, fatty alcohols, polyoxyethylene-9-lauryl ether, fatty acids such as palmitic acid or oleic acid, sorbitan trioleate, glycocholate, surfactin, poloxomers, sorbitan fatty acid esters such as sorbitan trioleate, tyloxapol, and phospholipids.

The nanoparticles can contain a plurality of layers. The layers can have similar or different release kinetic profiles for the compound. For example, the nanoparticles can have a controlled-release core surrounded by one or more additional layers. The one or more additional layers can include an instant-release layer, preferably on the surface of the nanoparticles. The instant-release layer can provide a bolus of the compound shortly after administration.

The composition and structure of the nanoparticles can be selected such that the nanoparticles are pH-sensitive or pH-responsive. In some forms, the particles are formed of pH-sensitive or pH-responsive polymers such as the enteric polymers commercially available from under the tradename EUDRAGIT®, as described above. Depending on the particle materials, the decomposition/degradation or structural change of the nanoparticles may occur at different locations of the gastrointestinal tract. In some embodiments, the particle materials are selected such that the pharmaceutical formulations can survive exposure to gastric acid and release the compound in the intestines after oral administration.

D. Controlled Release

In some embodiments, the pharmaceutical formulations can be controlled release formulations. Examples of controlled release formulations include extended-release formulations, delayed release formulations, pulsatile release formulations, and combinations thereof. In some embodiments, each dosage unit in capsule may contain a plurality of drug-containing beads, granules, or particles, having different release profiles.

1. Extended Release

In some embodiments, the extended-release formulations are prepared as diffusion or osmotic systems, for example, as described in “Remington—The science and practice of pharmacy” (20th Ed., Lippincott Williams & Wilkins, 2000).

A diffusion system is typically in the form of a matrix, generally prepared by compressing the drug with a slowly dissolving carrier, optionally into a tablet form. The three major types of materials used in the preparation of the matrix are insoluble plastics, hydrophilic polymers, and fatty compounds. Plastic matrices include, but are not limited to, methyl acrylate-methyl methacrylate copolymer, polyvinyl chloride, and polyethylene. Hydrophilic polymers include, but are not limited to, cellulosic polymers such as methyl ethyl cellulose, hydroxyalkylcelluloses (such as hydroxypropylcellulose, hydroxypropylmethylcellulose), sodium carboxymethylcellulose, CARBOPOL® 934, polyethylene oxides, and mixtures thereof. Fatty compounds include, but are not limited to, various waxes such as carnauba wax and glyceryl tristearate, wax-type substances including hydrogenated castor oil and hydrogenated vegetable oil, and mixtures thereof.

In some embodiments, the plastic material is a pharmaceutically acceptable acrylic polymer, including but not limited to, acrylic acid and methacrylic acid copolymers, methyl methacrylate copolymers, ethoxyethyl methacrylate copolymers, cyanoethyl methacrylate copolymers, aminoalkyl methacrylate copolymers, poly(acrylic acid), poly(methacrylic acid), methacrylic acid alkylamine copolymers, poly(methyl methacrylate), poly(methacrylic acidxanhydride), polymethacrylate, polyacrylamide, poly(methacrylic acid anhydride), and glycidyl methacrylate copolymers.

In some embodiments, the acrylic polymer can be an ammonio methacrylate copolymer. Ammonio methacrylate copolymers are well known in the art and are described as fully polymerized copolymers of acrylic and methacrylic acid esters with a low content of quaternary ammonium groups.

In some embodiments, the acrylic polymer is an acrylic resin lacquer such as those commercially available from under the tradename EUDRAGIT®. In some embodiments, the acrylic polymer contains a mixture of two acrylic resin lacquers, EUDRAGIT® RL30D and EUDRAGIT® RS30D. EUDRAGIT® RL30D and EUDRAGIT® RS30D are copolymers of acrylic and methacrylic esters with a low content of quaternary ammonium groups, the molar ratio of ammonium groups to the remaining neutral methacrylic esters being 1:20 in EUDRAGIT® RL30D and 1:40 in EUDRAGIT® RS30D. In some embodiments, the mean molecular weight for both copolymers is about 150,000. The code designations RL (high permeability) and RS (low permeability) refer to the permeability properties of these polymers. EUDRAGIT® RL/RS mixtures are insoluble in water and in digestive fluids. However, multiparticulate systems formed to include the same are swellable and permeable in aqueous solutions and digestive fluids. In some embodiments, the acrylic polymer can also be or include other EUDRAGIT® acrylic resin lacquers, such as EUDRAGIT® S-100, EUDRAGIT® L-100, or a mixture thereof.

The polymers described above such as EUDRAGIT® RL/RS may be mixed in any desired ratio in order to ultimately obtain a sustained-release formulation having a desirable dissolution profile. Desirable sustained release multiparticulate systems may be obtained, for instance, from 100% EUDRAGIT® RL, to 50% EUDRAGIT® RL+50% EUDRAGIT® RS, and to 10% EUDRAGIT® RL+90% EUDRAGIT® RS.

Matrices with different drug release mechanisms described above can be combined in a final dosage form containing single or multiple units. Examples of multiple units include, but are not limited to, multilayer tablets and capsules containing tablets, beads, or granules. An immediate release portion can be added to the extended-release system by means of either applying an immediate release layer on top of the extended-release core using a coating or compression process or in a multiple unit system such as a capsule containing extended and immediate release beads.

Extended-release tablets containing hydrophilic polymers are prepared by techniques commonly known in the art such as direct compression, wet granulation, or dry granulation.

Extended-release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Alternatively, extended-release formulations can be prepared using osmotic systems or by applying a semi-permeable coating to a solid dosage form. In the latter case, the desired drug release profile can be achieved by combining low permeable and high permeable coating materials in suitable proportion.

2. Delayed Release

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a coating material. The drug-containing composition may be a tablet for incorporation into a capsule, a tablet for use as an inner core in a “coated core” dosage form, or a plurality of drug-containing beads, particles, or granules, for incorporation into either a tablet or capsule. Suitable coating materials include bioerodible, gradually hydrolyzable, gradually water-soluble, and/or enzymatically degradable polymers, such as those described above. In some embodiments, the coating material is or contains enteric polymers. Combinations of different coating materials may also be used. Multilayer coatings using different polymers may also be applied.

The preferred coating weights for particular coating materials may be readily determined by those skilled in the art by evaluating individual release profiles for tablets, beads and granules prepared with different quantities of the coating materials.

The coating materials may contain conventional additives, such as plasticizers (generally represent about 10 wt % to 50 wt % relative to the dry weight of the coating material), colorants, stabilizers, glidants, etc., such as those described above.

3. Pulsatile Release

Pulsatile-release formulations release a plurality of drug doses at spaced-apart time intervals. Generally, upon administration, such as ingestion, of the pulsatile-release formulations, release of the initial dose is substantially immediate, e.g., the first drug release “pulse” occurs within about one hour of administration. This initial pulse is followed by a first time-interval (lag time) during which very little or no drug is released from the formulations, after which a second dose is then released. Similarly, a second lag time (nearly drug release-free interval) between the second and third drug release pulses may be designed. The duration of the lag times will vary depending on the formulation design, especially on the length of the desired drug administration interval, e.g., a twice daily dosing profile, a three times daily dosing profile, etc.

For pulsatile-release formulations providing a twice daily dosage profile, the nearly drug release-free interval has a duration of approximately 3 hours to 14 hours between the first and second dose. For dosage forms providing a three times daily profile, the nearly drug release-free interval has a duration of approximately 2 hours to 8 hours between each of the three doses.

In some forms, the pulsatile-release formulations contain a plurality of drug carriers with different drug-release kinetics.

In some forms, the pulsatile-release formulations contain a drug carrier with a plurality of drug-loaded layers. The drug-loaded layers may have different drug release kinetics. The layers may be separated by a delayed-release coating. For example, the carrier may have a drug-loaded layer on the surface for the first pulse and a drug-loaded core for the second pulse; the drug-loaded core may be surrounded by a delayed-release coating, which creates a lag time between the two pulses.

In some embodiments, the pulsatile release profile is achieved with formulations that are closed and preferably sealed capsules housing at least two drug-containing “dosage units” wherein each dosage unit within the capsule provides a different drug release profile. Control of the delayed release dosage unit(s) is accomplished by a controlled release polymer coating on the dosage unit, or by incorporation of the drug in a controlled release polymer matrix. Each dosage unit may comprise a compressed or molded tablet, wherein each tablet within the capsule provides a different drug release profile.

### V. Methods of Using

Disclosed are methods of enhancing synaptic function and/or treating a neurological condition or disorder in a subject in need thereof. The methods include administering an effective amount of a compound disclosed herein to the subject.

In some embodiments, the compound is administered in the form of a pharmaceutical formulation, such as those described above. The compound or pharmaceutical formulation can be administered in a variety of manners, depending on whether local or systemic administration is desired. In some embodiments, the compound is directly administered to a specific bodily location of the subject, e.g., topically administration. In some embodiments, the compound is administered in a systemic manner, such as enteral administration (e.g., oral administration) or parenteral administration (e.g., injection, infusion, and implantation). Exemplary administration routes include oral administration, intravenous administration such as intravenous injection or infusion, and topical administration. In some embodiments, the compound is administered orally. In some embodiments, the compound is administered intravenously. In some embodiments, the compound is administered intranasally.

In general, the neurological condition or disorder is mediated by abnormality in GluN3-containing NMDA receptors, including those containing GluN3A or GluN3B. The abnormality in GluN3-containing NMDA receptors can be caused by or associated with elevated expression levels and/or unregulated or under-regulated activity of these receptors.

Exemplary neurological conditions or disorders in this context include, but are not limited to, (1) major mental disorders (such as depression, bipolar disorder, attention-deficit disorder, schizophrenia, anxiety, various psychoses, and epilepsies), (2) conditions that involve basal ganglia or altered dopamine (such as dystonia, Huntingtin's disease, Parkinson's disease, and L-DOPA-induced dyskinesias or dyskinesias that result from medication), (3) substance abuse/addiction or predisposition to substance abuse/addiction, (4) pain disorders, (5) developmental delay or situations with impaired learning, memory, and/or cognition (such as neurodegenerative diseases, e.g., Alzheimer's disease, Parkinson's disease, and frontal lobe dementia, and motor retraining after acute injury), (6) acute neuronal or glial injuries (such as those that occur during hypoxia, ischemia, stroke, periventricular leukomalacia, traumatic brain injury, spinal cord injury, neonatal seizures, or status epilepticus), and (7) circuit disorders (such as Huntington's disease).

In some embodiments, the compound can be administered in combination with one or more additional pharmaceutically active agents, such as other medications for neurological diseases or disorders. The one or more additional pharmaceutically active agents can be formulated in the same pharmaceutical formulation as the compound. Alternatively, the one or more additional pharmaceutically active agents can be formulated in separate pharmaceutical formulation(s).

As used herein, “effective amount” of a material refers to a nontoxic but sufficient amount of the material to provide the desired result. The exact amount required may vary from subject to subject, depending on the species, age, and general condition of the subject, the severity of the disease, disorder, or condition that is being treated, the particular drug or therapy used, its mode of administration, and the like.

A. Physiological Relevance and Target Indications

NMDA receptors, including those that contain one or more GluN3 subunits, play a role in neurological functions including memory, learning (Tang, et al., Nature, 1999, 401, 63-69; Collingridge, et al., Neuropharmacology, 2013, 64, 13-26), and synaptic plasticity (Okabe, et al., J. Neurosci., 1998, 18, 4177-4188; Bliss, et al., Nature, 1993, 361, 31-39) and have long been connected to basic neurological function. They have been implicated in a wide range of neurological diseases and disorders. Both hypo- and hyper-function of NMDA receptors have been connected to Alzheimer's disease, Parkinson's disease, schizophrenia, depression, stroke, epilepsy, autism, developmental delay, intellectual disability, and psychosis, among others (Paoletti, et al., Nature Reviews, 2013, 13, 383-400; Traynelis, et al., Pharmacol. Rev., 2010, 62(3), 405-496; and Kemp, et al., Nat. Neurosci., 2002, 5, 1039-1042). There has thus been significant interest in developing NMDA receptor modulators as altered NMDA receptor activity has been implicated in schizophrenia (Moghaddam, Neuropsychopharmacology, 2012, 37, 4-15), autism (Won, et al., Nature, 2012, 486, 261-265; Schmeisser, et al., Nature, 2012, 486, 256-260), encephalitis (Hughes, et al., J. Neurosci., 2010, 30, 5866-5875) and age-related memory loss (Paoletti, et al., Nat. Rev. Neurosi., 2013, 14, 383-400).

Previous FDA-approved drugs that target NMDA receptors, e.g., memantine for moderate-to-severe Alzheimer's disease (Reisberg, et al., N. Engl. J. Med., 2003, 348, 1333-1341), non-selectively block the well-conserved ion pore irrespective of subunit composition, thereby leading to extensive neurological side effects.

Subunit-selective modulators of the NMDA receptors can reduce the side effect profile while maintaining the ability to alleviate receptor dysfunction and ultimately provide treatment for these conditions.

During development of the central nerve system (CNS), an overproduction of synapses yields networks of connected neurons, and the resulting circuit is refined by strengthening and stabilizing some synapses but eliminating others, yielding a circuit with sparse but highly specific and durable connections. These types of synaptic refinement are essential and are prominent during critical periods of development, when experience-dependent synapse refinement converts early patterns of connectivity into adult circuits that allow information processing.

GluN3A-containing NMDA receptors function to destabilize synapses, and hence have a role in tagging some synapses for elimination (or pruning), which is important for synaptic refinement and circuit function (Perez-Otano, et al., Nature Reviews, 2016, 17, 623-635). Normal downregulation of GluN3A-containing NMDA receptors starting at adolescence provides a developmental switch for the activity-dependent maturation and stabilization of selected synapses, and a growing number of studies links GluN3A dysregulation to major brain disorders.

Reduction or enhancement of GluN3A expression in mice through genetic strategies shows that GluN3A is a modulator of synapse maturation and stabilization. Specifically, Grin3a-knockout mice exhibit a large increase in the number of dendritic spines (and an enlargement in their size) in cortical pyramidal neurons (Das, et al., Nature, 1998, 393, 377-381), which can enhance synaptic NMDA receptor responses and enhance the developmental onset of long-term potentiation (LTP) (Roberts, et al., Neuron, 2009, 63, 342-356). Conversely, increasing the level of GluN3A decreases spine stability and promotes pruning without altering the rates at which new spines are formed (Kehoe, et al, J. Neurosci., 2014, 34, 9213-9221). Thus, GluN3A is involved in learning and memory, and influences all conditions that depend on excitatory synaptic transmission and thus specific circuit connectivity.

Modulation of either GluN3A- or GluN3B-containing NMDA receptors can sculpt neuronal circuit function, and thus is of utility in a broad range of neurological conditions or disorders that are derived from altered circuit function, including major mental disorders such as depression, bipolar disorder, attention-deficit disorder, schizophrenia, anxiety, various psychoses, as well as epilepsies. Negative modulation of GluN3-containing NMDA receptors is of utility for conditions that involve basal ganglia or altered dopamine, including dystonia, Huntingtin's disease, Parkinson's disease, and L-DOPA-induced dyskinesias or dyskinesias that result from medication. A predisposition to substance abuse or addiction also involves abnormal circuitry that can be modified by modulation of GluN3, as does a wide range of pain disorders in addition to the perception of pain.

Synaptic plasticity is well known to be controlled by NMDA receptors, and modulation of excitatory synapses and synaptic connectivity by inhibition of GluN3-containing NMDA receptors can alter synaptic plasticity, and be of use in developmental delay or situations with impaired learning, memory, and/or cognition, which include all neurodegenerative diseases such as Alzheimer's disease, Parkinson's disease, frontal lobe dementia, as well as in motor retraining after acute injury and motor coordination and function in general.

The response of nervous tissue to acute neuronal or glial injuries, as can occur during hypoxia, ischemia, stroke, periventricular leukomalacia, traumatic brain injury, spinal cord injury, neonatal seizures, or status epilepticus can be modulated by GluN3-containing receptors, as can the ultimate circuit response to such insults.

Aberrant or inappropriately timed upregulation for GluN3 subunits (either GluN3A or GluN3B) can lead to circuit disorder, and as such inhibitors of receptors containing GluN3 can be beneficial. For example, elevated GluN3 levels in patients with Huntington's disease may contribute to the disease progression, and reduction in GluN3A expression in animal models is shown to improve synaptic phenotype, improve cognitive symptoms, and delay neurodegeneration (Marco, et al., Nature Medicine, 2013, 19, 1030-1038; Mahfooz, et al., Neurobiol. Dis., 2016, 93, 47-56).

### EXAMPLES

The present disclosure will now be described in more detail with reference to the following non-limiting examples. It should be noted that the particular assays used in the examples section are designed to provide an indication of activity.

### General Information for Molecular Biology, Bioanalytical Assays, and Pharmacokinetics

### A. Molecular Biology and Cell Biology

cDNA encoding human wild-type (WT) GluN1-1a (GenBank accession numbers NP_015566, hereafter GluN1) and rat GluN1-1a (NP_058706.1) were synthesized and subcloned into pGEMHE plasmid; some experiments used the rat splice variant GluN1-4a (NP_001257531.1). Human GluN3A (NP_597702.2) and rat GluN3B (NP_579842.2) were synthesized and subcloned into SP6 plasmids. For most experiments, human GluN1 was co-expressed with human GluN3A, except for mutagenesis experiments where rat GluN1 was co-expressed with human GluN3A. Rat GluN1-4a was always expressed with rat GluN3B.

EU1180-438 was also tested at 20 μM for actions at AMPA (rat GluAlflip, rat GluA2flip-R607Q, human GluA3flip-L531Y, human GluA4flip-L505Y (Stem-Bach, et al., Neuron, 1998, 907-918)), rat kainate (GluK1), rat GABAA (al0272S), rat GABAC (ρ1), rat glycine (α1), rat nicotinic acetylcholine (nAChR, α1β1δγ, α4β2, α7), and purinergic (human P2X2) receptors expressed in Xenopus oocytes. The cDNA was linearized by restriction enzymes (Thermo Fisher Scientific, Lithuania), and the cRNA encoding the receptor subunits was synthesized from the linear template cDNA using the mMESSAGE mMACHINE kit (Invitrogen/Thermo Fisher Scientific, Lithuania). The α1β1δγ nAChR subunits were injected at a 1:1:1:1 ratio while α4 ρ2 nAChR subunits were injected at a 1:1 ratio. The cDNAs encoding GABA and glycine subunits were provided by Dr. D. Weiss (University of Texas Health Science Center at San Antonio). The cDNAs encoding nAChR subunits were provided by Drs. R. Papke (University of Florida) and S. Heinemann (Salk Institute). The cDNA encoding the purinergic receptor was provided by Dr. R. Hume (University of Michigan).

Responses in the presence of EU1180-438 were expressed as a percent of control calculated as the average of the responses before and after EU1180-438 exposure. Site-directed mutagenesis was performed using standard molecular biology protocols and verified by DNA sequencing (Eurofins MWG Operon, Huntsville, AL).

### B. Two-Electrode Voltage-Clamp (TEVC) Recordings

Defolliculated Xenopus laevis oocytes (stage V-VI) were obtained from Ecocyte BioScience (Austin, Texas), or prepared from commercially available Xenopus ovaries (Xenopus 1, Dexter, MI) as previously described (Hansen, et al., Mol. Pharmacol., 2013). A Drummond Nanoject II (Broomall, PA) was used to inject oocytes with cRNAs encoding GluN1 and GluN3 at a 1:3 ratio in RNase-free water. The cRNA was diluted as needed to give responses with amplitudes ranging between 200-2000 nA (0.2-10 ng total cRNA). Following cRNA injection, the oocytes were stored at 15-19° C. in Barth's solution that contained (in mM) 88 NaCl, 2.4 NaHCO₃, 1 KCl, 0.33 Ca(NO₃)₂, 0.41 CaCl₂), 0.82 MgSO₄, and 5 Tris-HCl (pH 7.4 with NaOH), supplemented with 1 μg/ml streptomycin (Invitrogen, Carlsbad, CA) and 100 μg/ml gentamicin (Fisher Scientific, Pittsburgh, PA). Recordings were performed 2-5 days following cRNA microinjection at room temperature (23° C.) using a two-electrode voltage-clamp amplifier (OC725, Warner Instrument, Hamilton, CT). The signal was low-pass filtered at 10-20 Hz (4-pole, −3 dB Bessel) and digitized at the Nyquist rate using PCI-6025E or USB-6212 BNC data acquisition boards (National Instruments, Austin, TX) using EasyOocyte (http://www.easyoocyte.io/). Oocytes were placed in a custom-made chamber and continuously perfused (2.5 ml/min) with oocyte recording solution containing (in mM) 90 NaCl, 1 KCl, 10 HEPES, 0.5-1.0 BaCl₂, and 0.01 EDTA (pH 7.4 with NaOH). Solutions were applied by gravity, and solution exchange was controlled through a rotary valve (Hamilton, Reno, NV). Recording electrodes were filled with 0.3-3.0 M KCl, and current responses were recorded at a holding potential of −40 mV.

Agonist concentration-effect curves were expressed as a percentage of the response to 1 mM glycine and fitted by

Response=maximum/(1+(EC₅₀/[concentration])^{nH}) (1)
where nH is the Hill slope, EC₅₀ is the concentration of agonist that produces a half-maximal response, and maximum is the maximal response predicted for saturating agonist.

The IC₅₀ values for the negative allosteric modulators were expressed as a percentage of the response to maximally effective concentration of agonist (1 mM glycine) in the absence of test ligand and fitted by

Response (%)=(100−minimum)/(1+([concentration]/IC₅₀)^{nH})+minimum (2)
where nH is the Hill slope, IC₅₀ is the concentration that produces a half-maximal effect, and minimum is the degree of residual inhibition at a saturating concentration of the negative allosteric modulator.

### C. Hippocampal Slice Preparation

All procedures were approved by the University of Montana Institutional Animal Care and Use Committee. Wild type C57BL/6J mice and 3A-KO mice (Das, et al., Nature, 1998, 377-381) (B6; 129X1-Grin3atm1Nnk, Jackson Laboratory, Stock No: 029974) were given access to food and water at libitum and housed together in one breeding pair per cage. Mice (aged P8-15) of both sexes were used in studies of glycine and NMDA responses in acute hippocampal slices. Transverse hippocampal slices were cut as previously described (Yi, et al., J. Physiol., 2014, 453-467). Briefly, immediately after euthanasia by deep anesthesia using isoflurane, the mouse was cardiac perfused with ice-old oxygenated high sucrose solution containing (in mM) 3 KCl, 24 NaHCO₃, 1.25 NaHzPO₄, 10 glucose, 230 sucrose, 0.5 CaCl₂), and 10 MgSO₄, saturated with 95% O₂/5% CO₂. When removed, the mouse brain was immediately submerged in ice-cold oxygenated cutting solution containing (in mM) 130 NaCl, 3 KCl, 24 NaHCO₃, 1.25 NaH₂PO₄, 10 glucose, 1 CaCl₂), and 3 MgSO₄. Transverse hippocampal slices (300 μm) were cut by a vibrating microtome (VT1200S, Leica Microsystems, Buffalo Grove, IL) and then incubated in the cutting solution at room temperature for at least one hour.

D. Whole-Cell Patch-Clamp Recordings from Hippocampal Neurons

A hippocampal slice was transferred to a recording chamber (RC-26GLP, Warner Instruments, Hamden, CT) mounted on a SliceScope Pro 2000 (Scientifica, Clarksburg, NJ). The slice was continuously perfused at a rate of 2-3 ml/min with oxygenated ACSF solution containing (in mM) 130 NaCl, 3 KCl, 24 NaHCO₃, 1.25 NaH₂PO₄, 10 glucose, 2 CaCl₂), and 1 MgSO₄, maintained at 32° C. using dual in-line and heated platform temperature control (TC-344A, Warner Instruments). Recording electrodes with a tip resistance of 2-4 MΩ were fabricated using a micropipette puller (P-1000, Sutter Instruments, Novato, CA) and filled with internal solution containing (in mM): 120 Cs-methanesulfonate, 4.6 MgCl₂, 10 HEPES, 15 BAPTA, 4 Na₂-ATP, 0.4 Na-GTP, 1 QX-314, and 10 K₂-creatine phosphate, pH 7.25, 280-290 mOsm. Whole-cell recordings were made using a Multiclamp 700B amplifier (Molecular Devices) with filtering at 4 kHz (Bessel) and digitized at 10 kHz using Digidata 1440A with the pCLAMP 10 software (Molecular Devices). For recordings of glycine-induced or NMDA-induced current responses, glycine (10 mM, 100 ms duration, 5 psi) or NMDA (0.5 mM, 15-30 ms duration, 5 psi) was delivered using a custom-made picospritzer (openspritzer; Forman, et al., Sci. Rep., 2017, 2188) every 30-60 s to the soma of patched cell through a borosilicate capillary tube (˜10 μM in tip diameter). For the recordings of glycine responses, the external solution was supplemented with 10 μM gabazine, 2 μM NBQX, 100 μM DL-APV, 50 μM strychnine to block GABAA, AMPA, GluN1/GluN2 NMDA, and glycine receptors respectively. After at least 5 min of stable baseline recordings, 1 μM CGP-78608 was bath-applied to the slice to prevent glycine-induced desensitization of GluN1/GluN3A NMDA receptors. Subsequently, test compound EU1180-438 at 30 μM or vehicle (0.15% DMSO) was added to the recording solution and bath-applied to the recorded cells from WT mice. For the recordings of NMDA responses, the external solution was supplemented with 10 μM gabazine, 2 μM NBQX, and 3 μM glycine. After at least 5 min stable baseline recordings, test compound EU1180-438 at 30 μM or vehicle (0.15% DMSO) was added to the recording solution and bath-applied to the recorded cells from WT mice for 10 min. In the end 400 μM DL-APV was applied for 5 min for full inhibition of GluN1/GluN2 NMDA receptors.

For all the recorded cells, series resistance (typically <20 MΩ) was monitored throughout the experiment with a 200 ms long, 5 mV hyperpolarizing voltage jump. The recording was excluded from analysis if series resistance changes of >20% were observed. For glycine and NMDA experiments in hippocampal slices, analyses of peak amplitudes were performed with Axograph (axograph.com). Data are shown as mean±SEM. Paired/unpaired t test were used for statistical comparisons as indicated, and P<0.05 was considered statistically significant.

Nonstationary variance analysis (Perszyk, et al., Nat. Chem. Biol., 2020, 16(2), 188-196) was conducted on the deactivation phase of the neuronal glycine-evoked current responses bathed in vehicle (control) or 30 μM EU1180-438 (V_{HOLD}: −60 mV). Ten individual responses to pressure-applied glycine were identified both during control and after inhibition by EU1180-438 had reached steady-state. The current was divided into 50 equally spaced segments, and the mean current amplitude of each segment was determined. The current was high pass filtered with a cutoff of 1 Hz, and the current variance was determined for 50 segments. The plot of variance vs. current for each response was fitted by the equation:

Variance=iI−I²/N
where I is the current amplitude of macroscopic response, i is the weighted mean unitary current, and N is the channel numbers. Virtually identical results were found if the current-variance relationship for all 10 responses for each cell were averaged and then fitted by the foregoing equation. Chord conductance y was determined assuming 0 mV for the reversal potential.

### E. Homology Modeling

Amino acids were numbered with the initiating methionine set to 1. A protein alignment was created for GluN1, GluN2, and GluN3A subunit sequences using the program Muscle (Edgar, BMC Bioinformatics, 2004, 113). Five GluN1/GluN3A receptor homology models made up of 3313 amino acids were generated using modeler 9v21 (ŠAli, et al., Protein Sci., 1994, 1582-1596) from three different templates (molpdf scores ranged from 113309-115258). The templates consisted of two diheteromeric GluN1/GluN2B receptor structures (PDB entries 4PE5 and 5FXH with resolution of 3.96 and 5 Å, respectively) and a monomer that represents the ABD domain of GluN3A (PDB entry 2RC7 with resolution of 1.58 Å). Regions of GluN3A for which there were no template structure coverage were removed during model building.

Quality analysis was performed using the PDBsum generator (Laskowski, Nucleic Acid Res., 2009, D355-D359), Schrödinger 2019-1 (Schrödinger Release 2019-1: Schrödinger Suite 2019-1 Protein Preparation Wizard; Epik, Schrödinger, LLC, New York, NY, 2016; Impact, Schrödinger, LLC, New York, NY, 2016; Prime, Schrödinger, LLC, New York, NY, 2019) and Modeler (ŠAli, et al., Protein Sci., 1994, 1582-1596). The model was prepared for analysis using the protein preparation wizard in which protonation states were assigned followed by an energy minimization cycle to relieve unfavorable constraints (Schrödinger Release 2019-1: Schrödinger Suite 2019-1 Protein Preparation Wizard; Epik, Schrödinger, LLC, New York, NY, 2016; Impact, Schrödinger, LLC, New York, NY, 2016; Prime, Schrödinger, LLC, New York, NY, 2019). The minimization cycle consisted of first minimizing only hydrogens, followed by a restrained minimization using imperf with a convergence of the RMSD of heavy atoms to 0.3 Å (Schrödinger Release 2019-1: Schrödinger Suite 2019-1 Protein Preparation Wizard; Epik, Schrödinger, LLC, New York, NY, 2016; Impact, Schrödinger, LLC, New York, NY, 2016; Prime, Schrödinger, LLC, New York, NY, 2019).

### F. In Vivo Analysis of Pharmacokinetic Properties

EU1180-438 was dosed at 20 mg/kg intraperitoneal in seven freely-fed male C57BL/6 mice. EU1180-438 was formulated in 5% DMA, 10% PEG, 30% HPBCD, qs to volume w/water.

Plasma was sampled at seven points: pre-dose, 0.25, 0.5, 1, 2, 4, and 8 hours (n=3 per time point, 21 plasma samples per treatment group). Brain samples were taken at four points: 0.25, 1, 2, and 4 hours (n=3 per time point, 12 brain samples per treatment group). Samples were analyzed via LC-MS/MS to quantify the amount of parent compound present in each sample.

### General Information for Chemical Reagents, Synthesis and Characterizations

The compound [(1S)-1-[[(7-bromo-1,2,3,4-tetrahydro-2,3-dioxo-5-quinoxalinyl)methyl]amino]ethyl] phosphonic acid hydrochloride (CGP-78608, Tocris) was prepared as a stock solution of 10 mM in 2.2 eq. NaOH solution. DL-APV, QX-314, NBQX and gabazine were purchased from Hello Bio. All the other regents were purchased from Sigma Aldrich, Oakwood Chemicals, Carbosynth, or other companies, and used directly without further purification. EU1180-438 was synthesized as described below and prepared in DMSO at stock concentrations of 20 mM.

Tetrahydrofuran (THF), dichloromethane (DCM), toluene, dimethylformamide (DMF) chloroform, pyridine, and dimethylsulfoxide (DMSO) were used from DRISOLV® under inert conditions using argon gas. Reactions requiring anhydrous conditions were performed under argon atmosphere.

Chemical reactions were generally monitored by thin layer chromatography (TLC) on precoated aluminum plates (silica gel 60 F254, 0.25 mm) or LC-MS on an Agilent Technologies 1200 series instrument. TLC visualization was achieved with a UV lamp, KMNO₄ stain, CAM stain, or p-anisaldehyde stain. LC-MS analysis was performed on an Agilent 1200 series instrument coupled to a 6120 quadrupole mass spectrometer (ESI-API), with UV detection at 254 and 210 nm and an Agilent Zorbax™ XDB-18 C18 column (50 mm×4.6 mm, 3.5 μm). Purification by flash column chromatography was performed using a Teledyne ISCO CombiFlash™ Companion instrument with Teledyne Redisep™ normal phase columns.

¹H and ¹³C NMR spectra were recorded on a 300 MHz Varian VNMRS (75 MHz for ¹³C), a 400 MHz Varian INOVA (101 MHz for ¹³C), a 400 MHz Varian VNMRS, a 500 MHz Varian INOVA (126 MHz for ¹³C) or a 600 MHz Varian INOVA (150 MHz for ¹³C). CDCl₃, DMSO-d₆ and MeOD-d₄ were used as standard deuterated solvents. The chemical shifts were reported in ppm and referenced to the residual deuterated solvent. The coupling constants, J, were reported in Hertz (Hz). High resolution mass spectra were recorded on a VG 70-S Nier Johnson or JEOL instrument by the Emory University Mass Spectroscopy Center.

The maximum solubility for the synthesized compounds was determined using a BMG Labtech Nephelostar™ nephelometer according to the manufacturer's instructions. For example, EU1180-438 was taken up in DMSO to prepare a 10 mM stock solution. The 10 mM solution was serially diluted into eleven concentrations (10, 5, 2.5, 1.25, 0.625, 0.3125, 0.1563, 0.0781, 0.0391, 0.0193, and 0.0098 mM) at the top of a clear-bottom 96-well plate. These were then each distributed into the subsequent 7 wells in 2.5 μL increments and diluted 100-fold (2.5 μL to 250 μL) with oocyte recording buffer (pH 7.4) into concentrations of 100, 50, 25, 12.5, 6.25, 3.125, 1.563, 0.781 0.391, 0.193, and 0.098 μM. The 96-well plate was shaken gently for 90 minutes before analysis. A segmental regression showed that the maximum solubility of EU1180-438 is 37 μM.

Below is a list of general synthetic procedures used in preparing the compounds in the Examples below.

General Procedure A—The starting acetophenone (1 eq.), ammonium acetate (10 eq.), and sodium cyanoborohydride (6 eq.) were combined in a round bottom flask equipped with a stir bar and dissolved in methanol. After stirring the reaction under reflux for 24 hours, the methanol was removed under reduced pressure, the crude residue was reconstituted in ethyl acetate, and the organic phase was washed with 1 M disodium citrate (aq.). The organic layer was decanted, dried over anhydrous Na₂SO₄, filtered, and concentrated under reduced pressure to a viscous oil. The product was then purified by flash column chromatography (silica gel, 0-100% ethyl acetate in hexanes) to afford the pure racemic secondary amine as a viscous oil.

General Procedure B—The starting acetophenone (1 eq.) and ammonium formate (10 eq.) were dissolved in absolute ethanol in a round bottom flask equipped with a stir bar. After stirring the reaction solution at 80° C. for one hour, sodium cyanoborohydride (6 eq.) was added, and the mixture was stirred at 80° C. overnight. The solvent was removed under reduced pressure, the crude residue was reconstituted in ethyl acetate, and the organic phase was washed with aqueous saturated sodium carbonate. The organic layer was decanted, dried over anhydrous Na₂SO₄, filtered, and concentrated under reduced pressure to a viscous oil. The product was then purified by flash column chromatography (silica gel, 0-100% ethyl acetate in hexanes) to afford the pure racemic secondary amine as a viscous oil.

General Procedure C—A solution of the amine (1 eq.) in methanol was supplemented with triethylamine (2.1 eq.) and the methyl 2-(bromomethyl)benzoate substrate (1.2 eq.) and stirred under reflux overnight. The methanol was removed under reduced pressure, the crude residue in dichloromethane was loaded onto a column (silica gel), and the product was purified by flash column chromatography (0-30% ethyl acetate in hexanes) to afford the pure product as a sticky colorless oil/white solid.

General Procedure D—A solution of the amine (1 eq.) in ethanol was supplemented with N,N-diisopropylethylamine (DIPEA, 2.5 eq.) and the methyl 2-(bromomethyl)benzoate substrate (1.2 eq.) and stirred under reflux overnight. The ethanol was removed under reduced pressure, the crude residue in dichloromethane was loaded onto a column (silica gel), and the product was purified by flash column chromatography (0-30% ethyl acetate in hexanes) to afford the pure product as a sticky colorless oil/white solid.

General Procedure E—A solution of the amine (1 eq.) in toluene was supplemented with N,N-diisopropylethylamine (DIPEA, 2.5 eq.) and the phthalic anhydride (1.2-2.0 eq.) and stirred at 110° C. overnight. The toluene was removed under reduced pressure, the crude residue in dichloromethane was loaded onto a column (silica gel), and the product was purified by flash column chromatography (0-20% ethyl acetate in hexanes) to afford the pure product as a sticky colorless oil/white solid.

General Procedure F—The starting amine (1 eq.) and 4-(trifluoromethyl)phthalic acid (1.2 eq.) were suspended in glacial acetic acid and stirred at 80° C. overnight, eventually becoming a homogeneous solution after approximately 20 minutes. After removing the acetic acid under a steady stream of argon, the crude residue was loaded onto a column (silica gel) with dichloromethane, and the product was purified by flash column chromatography (0-20% ethyl acetate in hexanes) to afford the pure product as a sticky colorless oil/white solid.

General Procedure G—The synthetic method for the synthesis of the final compounds containing an isoindolinone moiety was performed using the following procedure. The substituted benzylic secondary amine, halogenated (or halogen free) methyl 2-(bromomethyl)benzoate (1.2 eq. to 1.3 eq.), DIPEA (3 eq.), and anhydrous methanol were sealed in a reaction vial and stirred at 40° C. for a period ranging from 4 hours to overnight, where it was observed that the majority of the benzylic amine had been alkylated. The temperature was increased to 75-80° C. and stirred for a period ranging from 4 hours to overnight at that temperature. The reaction mixture was directly immobilized onto silica gel, and the product was purified via flash column chromatography using a 0-50% EtOAc/hexane gradient eluent to afford the desired cyclized product as a colorless oil or white solid.

### Example 1. Synthesis and Characterization of 2-(2-(4-methoxyphenoxy)-1-(4-methoxyphenyl)ethyl)isoindolin-1-one (EU1180-438)

EU1180-438 was synthesized according to the steps shown in Scheme 1 below.

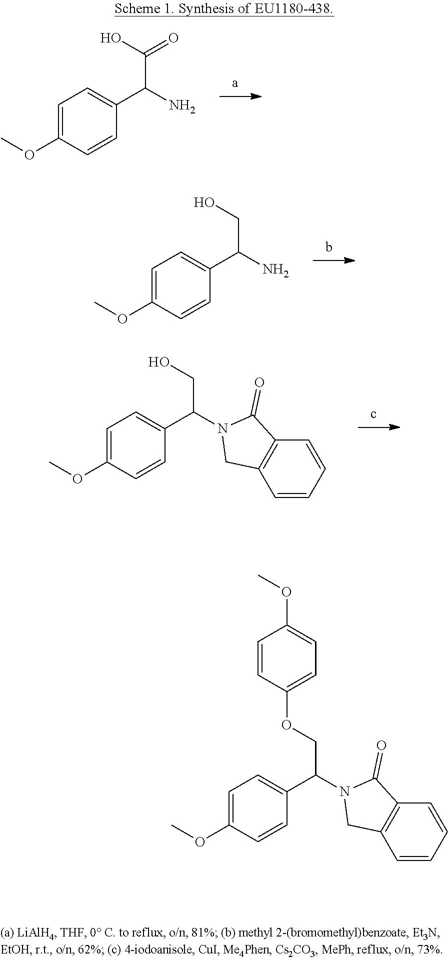

### A. Synthesis of 2-amino-2-(4-methoxyphenyl)ethan-1-ol

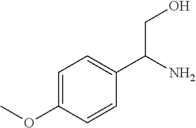

Lithium aluminum hydride (13.8 mL, 27.6 mmol, 2 M in THF, 5 eq.) was added to THF (31 mL, ˜0.18 M), cooled to 0° C., and 2-amino-2-(4-methoxyphenyl)acetic acid (1.00 g, 5.52 mmol, 1 eq.) was added in small portions. The solution was stirred at 0° C. for 1 hour and then brought to reflux overnight. Upon completion, the reaction was brought to 0° C. and 1.05 mL of water, 1.05 mL of 15% NaOH, and 3.15 mL of water were added dropwise in succession. The mixture was stirred until a white suspension was observed. The precipitate was filtered and the combined THF extracts were concentrated, brought up in DCM, washed with brine, dried over MgSO₄, and concentrated in vacuo. The crude product was purified via flash column chromatography (ISCO, Redisep 24 g column, solid load, 0-20% DCM/[MeOH w/6% v/v 7 M NH₃ in MeOH] gradient) to afford the title compound as a white solid (0.75 g, 81%).

### B. Synthesis of 2-(2-hydroxy-1-(4-methoxyphenyl)ethyl)isoindolin-1-one

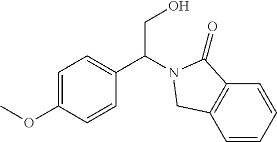 [SMILES: COc1ccc(C(CO)N2Cc3ccccc3C2=O)cc1]

2-amino-2-(4-methoxyphenyl)ethan-1-ol (0.20 g, 1.2 mmol, 1 eq.) was added to methyl 2-(bromomethyl)benzoate (0.33 g, 1.4 mmol, 1.2 eq.) and triethylamine (0.33 mL, 2.4 mmol, 2 eq.) in EtOH (6.0 mL, ˜0.20 M) and stirred at room temperature overnight. The reaction was quenched with water, extracted with DCM (3×), washed with brine, dried over MgSO₄, and concentrated in vacuo. The crude product was purified via flash column chromatography (ISCO, Redisep 12 g column, 0-100% EtOAc/hexanes gradient) to afford the title compound as a white foam (0.21 g, 62%).

### C. Synthesis of 2-(2-(4-methoxyphenoxy)-1-(4-methoxyphenyl)ethyl)isoindolin-1-one

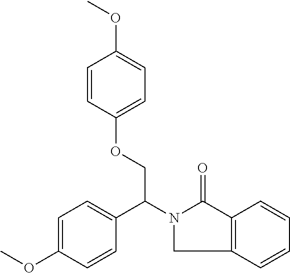 [SMILES: COc1ccc(OCC(c2ccc(OC)cc2)N2Cc3ccccc3C2=O)cc1]

An oven-dried microwave vial was charged with an oven-dried stir bar, 2-(2-hydroxy-1-(4-methoxyphenyl)ethyl)isoindolin-1-one (100 mg, 0.350 mmol, 1 eq.), copper(I) iodide (13 mg, 0.071 mmol, 0.2 eq.), 3,4,7,8-tetramethyl-1,10-phenanthroline (17 mg, 0.071 mmol, 0.2 eq.), 1-iodo-4-methoxybenzene (124 mg, 0.529 mmol, 1.5 eq.), and cesium carbonate (173 mg, 0.529 mmol, 1.5 eq.). The vessel was then capped and evacuated and back-filled with dry argon 3 times. Dry toluene (0.71 mL, ˜0.5 M) was then added, the vial evacuated and back-filled with dry argon once more and the reaction stirred at 110° C. for 48 hours. The reaction mixture was then cooled to room temperature, diluted with EtOAc, and filtered through a plug of celite, washing with additional EtOAc. The crude material was then concentrated and purified via flash column chromatography (ISCO, Redisep 4 g column, 0-100% EtOAc/hexanes gradient) to afford the title compound as a yellow foam (0.10 g, 73%). R_{f} (1:1 EtOAc:Hex): 0.48; ¹H NMR (500 MHz, CDCl₃) δ 7.89 (d, J=7.4 Hz, 1H), 7.50 (td, J=7.4, 1.3 Hz, 1H), 7.45 (td, J=7.4, 0.9 Hz, 1H), 7.40-7.34 (m, 3H), 6.91-6.85 (m, 4H), 6.84-6.80 (m, 2H), 5.87 (t, J=5.8 Hz, 1H), 4.58 (dd, J=10.0, 6.6 Hz, 1H), 4.54 (d, J=16.9 Hz, 1H), 4.48 (dd, J=10.0, 5.2 Hz, 1H), 4.24 (d, J=17.0 Hz, 1H), 3.79 (s, 3H), 3.75 (s, 3H). ¹³C NMR (126 MHz, CDCl₃) δ 168.66, 159.26, 154.29, 152.46, 141.72, 132.61, 131.33, 129.38, 129.13, 127.92, 123.80, 122.74, 115.83, 114.72, 114.17, 68.75, 55.71, 55.27, 53.35, 47.79. HRMS calcd. for C₂₄H₂₄O₄N, 390.16998 [M+H]⁺; found 390.17026 [M+H]⁺.

Purity was determined by LC-MS using an Agilent pump on a Zorbax XBD-C18 column (4.6 mm×50 mm, 3.5 μm). Method 1: 85-95% MeOH in water over 5 min at 1 mL/min (retention time=0.73 min). Method 2: 75-95% MeOH in water over 3 min at 1 mL/min (retention time 1.05 min). The purity of EU1180-438 was determined to be >95% under both methods. The solubility of EU1180-438 in the oocyte recording buffer (pH 7.4) was determined to be 37 μM, using methods described above.

### Example 2. Separation of EU1180-438 Enantiomers

Semipreparative separation of EU1180-438 enantiomers from racemic EU1180-438 (0.020 g) was done using a Kromasil 5-AmyCoat column (30 mm×250 mm) with the following conditions: 20 mL/min flow rate, 10 mL injection volume (2 mg/l mL), 90% hexanes/10% IPA over 150 min to afford (−)-EU1180-438 (tR 72.8 min) and (+)-EU1180-438 (tR 92.0 min). The enantiomeric excess (ee) was determined using an Agilent pump on a Kromasil 5-AmyCoat column (4.6 mm×150 mm, 5 μm) with the following conditions: 1 mL/min flow rate, 10 μL injection volume, 90% hexanes/10% IPA. (−)-EU1180-438: tR 38.1 min; >99% ee; [α]D20=−36 (c 0.10, dry CHCl₃). (+)-EU1180-438: tR 33.0 min, >99% ee; [α]D20=+37 (c 0.10, dry CHCl₃).

### Example 3. EU1180-438 Inhibits GluN1/GluN3 Receptors

Introduction of two mutations in the orthosteric glycine binding pocket of GluN1, F484A and T518L (hereafter GluN1^{FA,TL}), alters the glycine concentration-response profile for GluN1/GluN3 receptors by completely abolishing the inhibitory component caused by glycine binding to GluN1 in GluN1^{FA,TL}/GluN3 receptors (Kvist, et al., Neuropharmacology, 75, 324-336). Thus, GluN1^{FA,TL}/GluN3 receptors, which contain a GluN3 subunit that is virtually incapable of binding glycine, exhibit conventional concentration-response relationship that reaches maximal steady-state response, allowing determination of the glycine EC₅₀ values and thus potential assessment of GluN3-selective ligands (Kvist, et al., Neuropharmacology, 75, 324-336).

EU1180-438 was shown to be an inhibitor of recombinant GuN1^{FA,TL}/GluN3A receptors with no detectable activity toward recombinant GluN1/GluN2 NMDA receptors (including those containing GluN2A, GluN2B, GluN2C, or GluN2D).

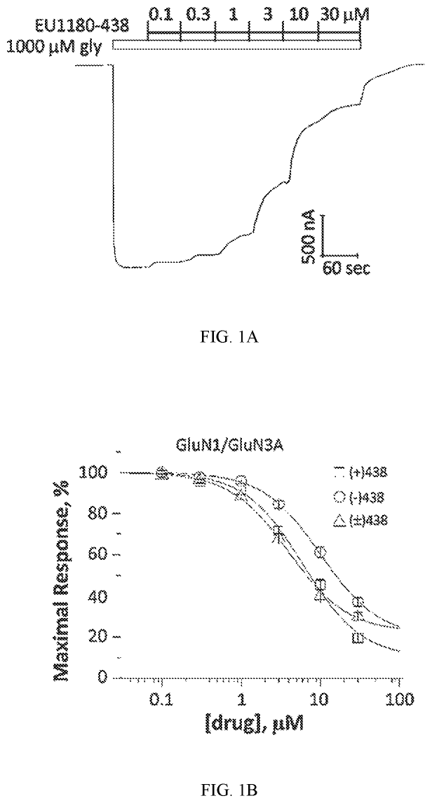

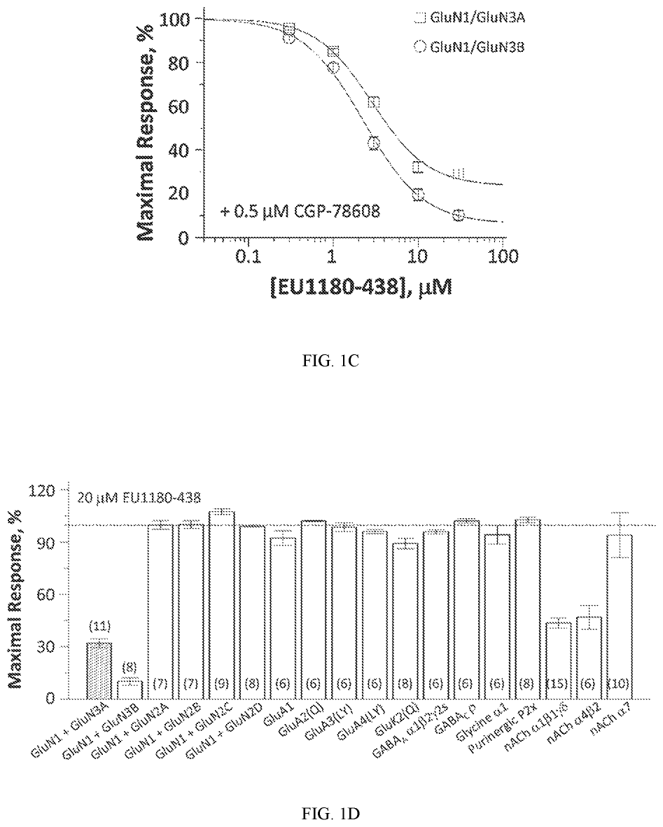

To evaluate inhibition of GluN1/GluN3 receptors by EU118N438, we established the concentration-effect relationship using a racemic mixture of EU1180-438, which inhibited GluN1^{FA,TL}/GluN3A receptors by 82% at a saturating concentration with an IC₅₀ value of 3.5 μM (FIGS. 1A and 1B and Table 1). The two enantiomers of EU1180-438 showed similar potency and effects toward GluN1^{FA,TL}/GluN3A receptors (FIG. 1B and Table 1).

TABLE 1
Pharmacological activities of EU1180-438 toward GluN1/GluN3 receptors

| | GluN1^{FATL}/GluN3A | GluN1/GluN3A + CGP ^{b} | GluN1/GluN3B + CGP ^{b} |
|---|---|---|---|
| | EC₅₀, (95% CI) | EC₅₀, (95% CI) | EC₅₀, (95% CI) |
| | % Inhibition^{a}, (n) | % Inhibition^{a}, (n) | % Inhibition^{a}, (n) |
| (±) EU1180-438, pH 7.4 | 3.5 μM, (2.8-3.8) | 1.8 μM, (1.5-2.1) | 2.2 μM, (1.9-2.6) |
| | 82%, (n = 18) | 85%, (n = 9) | 93%, (n = 8) |
| (+) EU1180-438, pH 7.4 | 7.1 μM, (5.7, 7.7) | ND | ND |
| | 96%^{a}, (n = 22) | | |
| (−) EU1180-438, pH 7.4 | 13 μM, (9.0-14) | ND | ND |
| | 84%^{a}, (n = 20) | | |
| (±) EU1180-438, pH 6.8 | 4.2 μM, (3.0-5.2) | ND | ND |
| | 72%^{a}, (n = 7) | | |

Data are shown as mean with 95% confidence interval given in parentheses, determined from mean of log(EC₅₀). For experiments involving EU1180-438, the percent response in saturating ligand is given, and (n) is the number of oocytes. Holding potential was −40 mV. Receptors contained human GluN1^{FATL}/GluN3A or rat GluN1-4a/GluN3B.
^{a}Percentage inhibition determined from fitted maximum at saturating test compound, calculated as 100 × (1 − I_{DRUG}/I_{CONTROL})
^{b} Experiments were performed in 0.5 μM GCP-78608
ND is not determined.

### Example 4. EU1180-438 is Selective for GluN1/GluN3 Receptors

The binding of glycine to the GluN1 subunits of the GluN1/GluN3 receptors appears to cause auto-inhibition by promoting entry into a desensitized state. Paoletti and colleagues (Grand, et al., Nat. Commun., 2018, 4769) therefore tested glycine site antagonists for their ability to enhance activation of GluN1/GluN3 by glycine by selectively preventing glycine binding to the GluN1 subunit. CGP-78608 was found to strongly enhance GluN1/GluN3A current responses, converting small and rapidly desensitizing currents from wild type GluN1/GluN3A into large and stable current responses. We thus evaluated the effects of EU1180-438 on human GluN1/GluN3A and rat GluN1/GluN3B (wild-type rat GluN1-4a/GluN3B) receptors activated in the presence of 500 nM CGP-78608, and found 85% maximal inhibition with an IC₅₀ of 1.8 μM at GluN1/GluN3A and 93% maximal inhibition with an IC₅₀ of 2.2 μM at GluN1/GluN3B, respectively (FIG. 1C, Table 1). In addition, rat GluN1-4a^{FA,TL}/GluN3B receptors, which contain a GluN1 splice variant that has been suggested to increase GluN3-mediated current amplitudes, were also inhibited by 82% at saturating concentrations with an IC₅₀ value of 5.2 μM (95% CI 4.4-5.9, n=6). EU1180-438 (20 μM) showed virtually no activity at GluN1/GluN2 NMDA receptors (rat GluN1-1a/GluN2A-D was used), GluA1-4 AMPA receptors, GluK2 kainate receptors, GABA receptors, or glycine receptors; EU1180-438 inhibited some, but not all, nicotinic receptors (FIG. 1D). These data demonstrate that EU1180-438 is a useful tool for exploration of GluN1/GluN3 activity in neuronal preparations.

### Example 5. EU1180-438 Inhibits Neuronal Responses Mediated by GluN3A-Containing Receptors

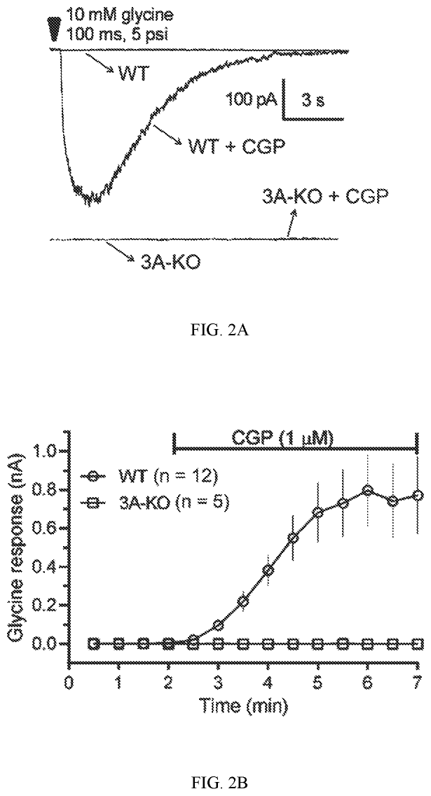

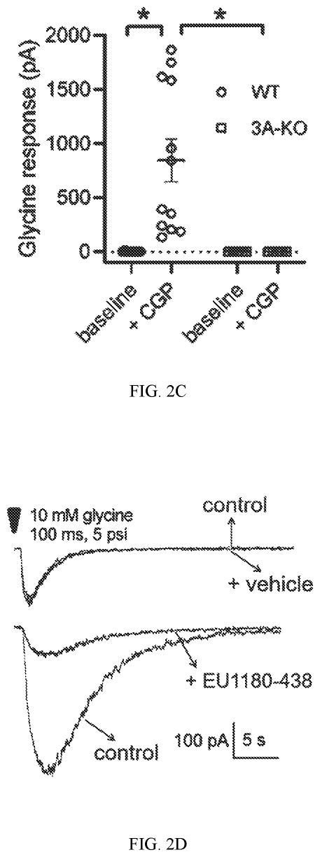

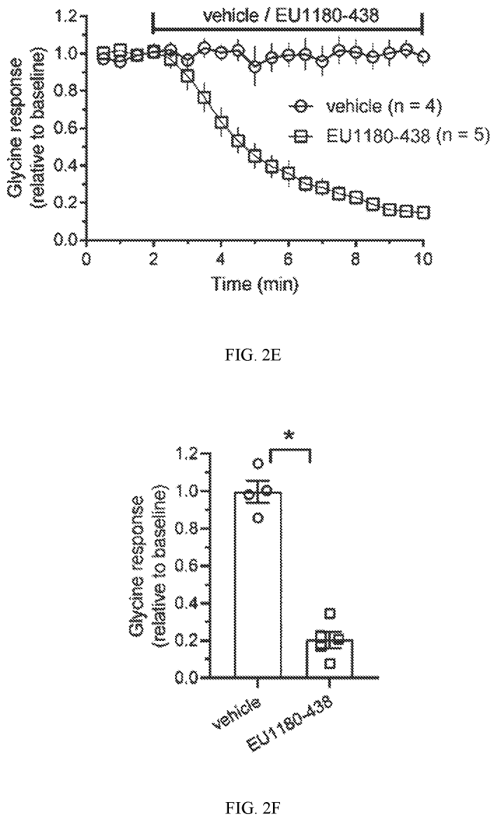

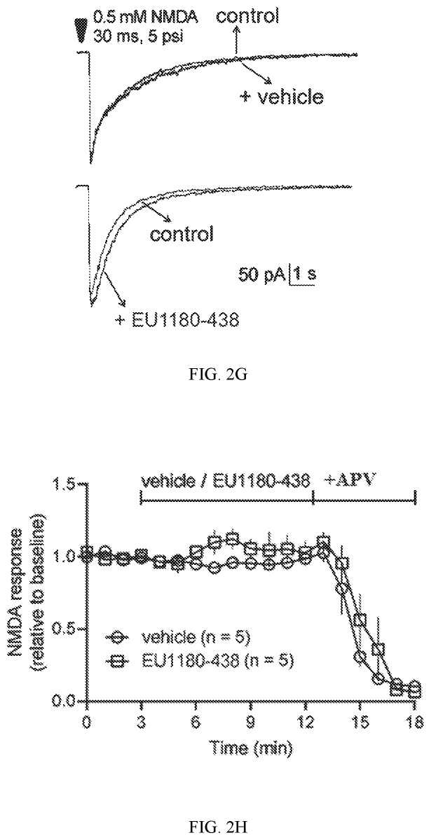

To evaluate the effects of EU1180-438 on responses from neuronal GluN3A-containing NMDA receptors, we first reproduced the recent evidence for native GluN1/GluN3A receptors expressed in hippocampal CA1 pyramidal cells (Grand, et al., Nat. Commun., 2018, 4769). Whole-cell recordings from hippocampal CA1 pyramidal cells in acute mouse brain slices (P8-15) were performed in the presence of strychnine, gabazine, NBQX, and APV to block inhibitory glycine receptors, GABAA receptors, AMPA/kainate receptors, and GluN1/GluN2 NMDA receptors, respectively (FIGS. 2A-2C). The presence of APV and CGP-78608 inhibits all “conventional” GluN1/GluN2 NMDA receptors. Responses were activated by brief pressure-induced application (hereafter puffs) of 10 mM glycine to the neuronal cell body in the absence and presence of bath-applied CGP-78608 (FIGS. 2A-2C). The glycine responses were abolished in slices from GluN3A-deficient mice (3A-KO) (FIGS. 2A-2C). These results demonstrate expression of native GluN1/GluN3A receptors in hippocampal CA1 pyramidal cells and show that these receptors are incapable of mediating glycine-activated current responses in the absence of a GluN1-selective antagonist.

Next, allosteric modulation by EU1180-438 of neuronal current responses was evaluated from native GluN1/GluN3A receptors in the presence of 1 μM CGP-78608 (FIGS. 2D-2F). EU1180-438 (30 μM) produced a strong inhibition of the glycine-activated responses compared to control responses in the absence of modulator (P<0.05; paired t-test) (FIGS. 2D-2F). By contrast, addition of vehicle (0.15% DMSO) did not affect glycine-activated responses from native GluN1/GluN3A receptors (P>0.05; paired t-test) (FIGS. 2D-2F).

To further establish the selectivity of EU1180-438 for GluN3-containing NMDA receptors, we investigated potential effects on responses activated by pressure application of 0.5 mM NMDA onto the hippocampal CA1 pyramidal cells (FIGS. 2G-2I). These responses, which are mediated by GluN1/GluN2 NMDA receptors, were not modulated by EU1180-438 compared to control in the absence of modulator (P>0.05; paired t-test) or compared to vehicle (0.15% DMSO; P>0.05; unpaired t-test) (FIGS. 2G-2I). Thus, EU1180-438 is capable of producing strong inhibition of native GluN1/GluN3A receptors in acute hippocampal slices with high selectivity for GluN1/GluN3 over GluN1/GluN2 NMDA receptors.

### Example 6. EU1180-438 is a GluN1/GluN3 Negative Allosteric Modulator

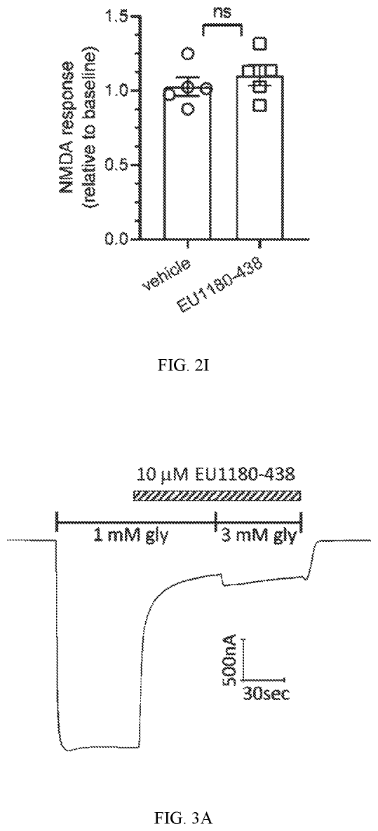

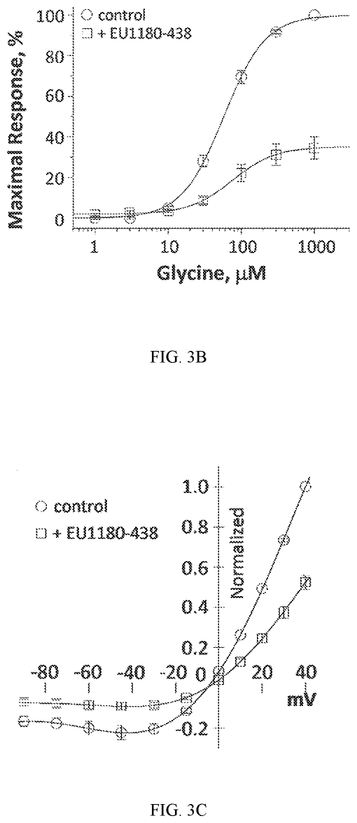

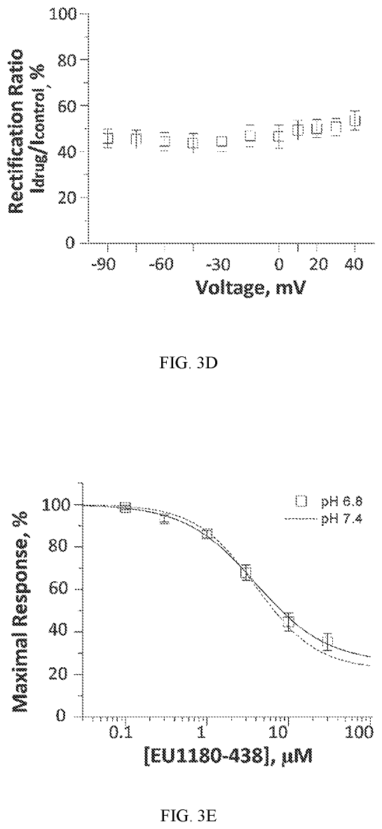

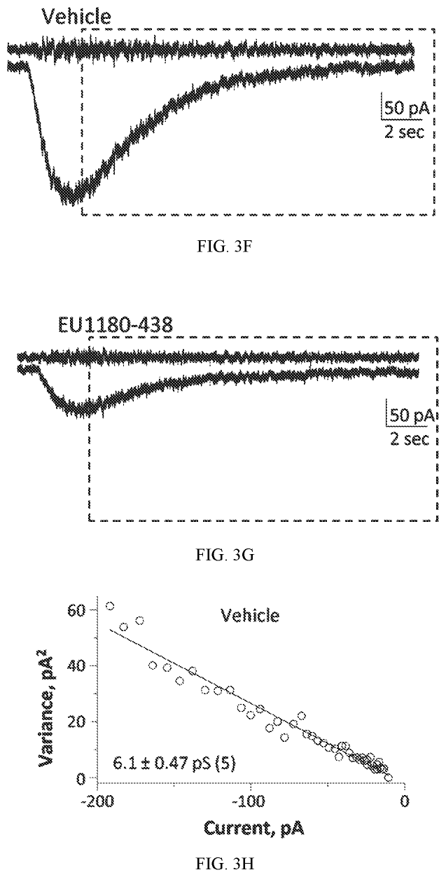

To determine the mechanism of action for EU1180-438 inhibition, experiments were performed to determine whether inhibition could be surmounted by increasing the glycine concentration. The inhibition of GluN1^{FA,TL}/GluN3A by EU1180-438 was not altered by increasing glycine concentration, consistent with a non-competitive mechanism of action (FIG. 3A). The response in the presence of 10 μM EU1180-438 was 29±4.2% of control in 1 mM glycine, and 29±4.2% of control in the presence 3 mM glycine (n=10). We subsequently recorded glycine concentration-response curves in the absence and presence of 30 μM EU1180-438 at GluN1^{FA,TL}/GluN3A receptors (FIG. 3B). The glycine EC₅₀ in the presence of EU1180-438 was 71 μM at GluN1^{FA,TL}/GluN3A and the 95% CI determined from log EC₅₀ was 50-84 μM (n=10), compared to an EC₅₀ value of 61 μM (95% CI, 46-71 μM, n=10) observed in the absence of EU1180-438. We also recorded the current-voltage (I-V) relationship for glycine-activated currents in the absence and presence of 4 μM EU1180-438 (FIG. 3C). Analysis of the I-V relationship revealed a similar degree of inhibition of the current response by EU1180-438 at all voltages (FIG. 3D), which we interpret as evidence that the inhibition does not reflect voltage-dependent channel block. These data demonstrate that EU1180-438 is a negative allosteric modulator of GluN1/GluN3 receptors.

Reduced extracellular pH has been shown to increase GluN1/GluN3A-mediated current amplitudes (Cummings and Popescu, Sci. Rep., 2016, 6, 23344). We tested whether EU1180-438 can inhibit currents elicited by glycine at pH 6.8. We found that EU1180-438 inhibited GluN1^{FA,TL}/GluN3A responses with an IC₅₀ value of 4.2 μM, with maximal inhibition of 72% at saturating concentrations of EU1180-438 (n=7, FIG. 3E, Table 1), compared to an ICso value of 3.5 μM at pH 7.4. These data suggest that EU1180-438 can inhibit GluN1/GluN3A responses during activity-dependent or ischemia-dependent changes in extracellular pH.

Some allosteric modulators with molecular determinants in the pre-M1 can reduce single channel conductance for glutamate receptors (Perszyk, et al., Nat. Chem. Biol., 2020, 16(2), 188-196; Yuan, et al., ACS Med. Chem. Lett., 2019, 10 (3), 237-242). To determine if EU1180-438 also changes unitary conductance of GluN3A-containing receptors, we performed nonstationary noise analysis on neuronal current responses (FIGS. 3F and 3G) to estimate unitary current of GluN1/GluN3A channels from the relationship between the macroscopic current amplitude and variance. The average chord conductance of GluN1/GluN3A channels that give rise to the macroscopic response in the presence of EU1180-438 showed no significant difference compared to the vehicle (vehicle: 6.1±0.47 pS, EU1180-438: 6.3±0.90 pS; n=5, p=0.78, paired t-test; FIGS. 3H and 3I). These data show that inhibition of GluN3A-containing receptor current by EU1180-438 does not involve changes in channel conductance.

### Example 7. Site of Action for EU1180-438

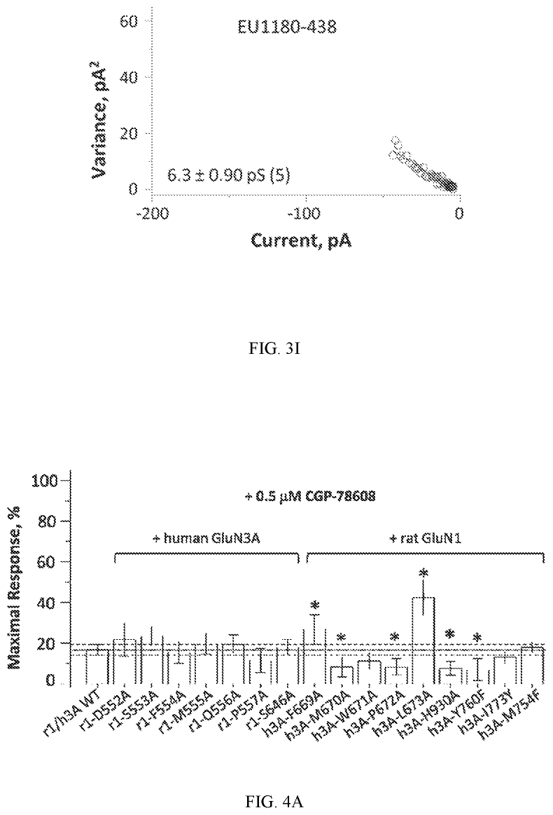

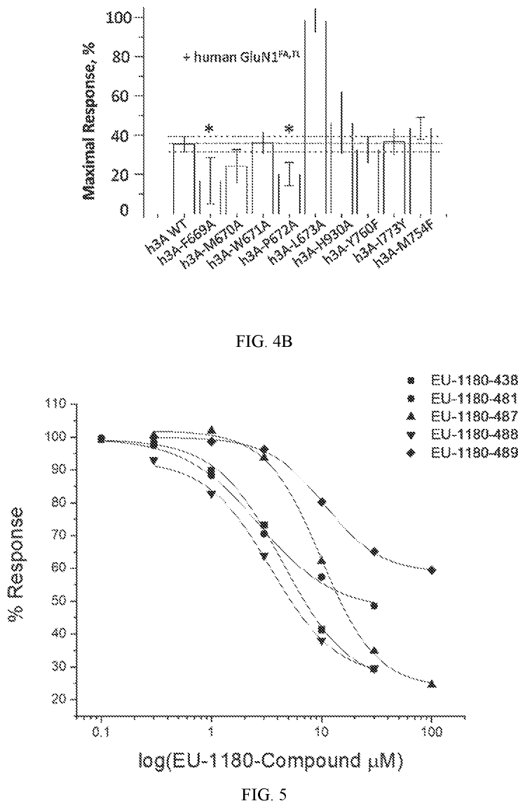

Ionotropic glutamate receptors harbor a binding site for modulators at the interface of the transmembrane region and the agonist binding domain (Perszyk, et al., Nat. Chem. Biol., 2020, 16(2), 188-196; Ogden and Traynelis, Mol. Pharmacol., 2013, 83(5), 1045-1056). This site appears promiscuous and has been shown by crystallography of AMPA receptors to bind distinct scaffolds within the same binding site (Yelshanskaya, et al., Neuron, 2016, 91 (6), 1305-1315). The latter pockets are located between the pre-M1 helix, the extracellular end of the M3 transmembrane helix, and parts of the M4 linker (Yelshanskaya, et al., Neuron, 2016, 91 (6), 1305-1315; Ogden and Traynelis, Mol. Pharmacol., 2013, 83(5), 1045-1056). At present, NMDA receptor structures have not been resolved with modulators bound to this region. We have identified mutations affecting the binding of other modulators within this region (Ogden and Traynelis, Mol. Pharmacol., 2013, 83(5), 1045-1056). We therefore performed mutagenesis studies to narrow down the potential binding pocket for EU1180-438. Site-directed mutagenesis was performed in proximity to the pre-M1 region of GluN1^{FA,TL} and GluN3A, and responses were recorded from mutant GluN1^{FA,TL}/GluN3A receptors. Pre-application of the GluN1-selective glycine site competitive antagonist, CGP-78608, “unmasks” glycine-activated responses from native GluN1/GluN3A receptors by preventing desensitization. We initially determined the effect of all mutations in the presence of CGP-78608 (FIG. 4A). There were no detectable effects of any GluN1 mutations on EU1180-438 inhibition, but 6 out of 9 GluN3A mutations perturbed the effects of EU1180-438 (FIG. 4A). We repeated analysis of these mutations in the absence of CGP-78608, but using the GluN1^{FA,TL} subunit to prevent desensitization, and found similar results for adjacent residues GluN3-P672 and GluN3-L673, which line a cavity that resides above the pre-M1 helix (FIG. 4B). These data are compatible with structural determinants of action for EU1180-438 in the pre-M1 region of GluN3A rather than GluN1. The cavity in GluN3A lined by residues affecting EU1180-438 inhibition was determined, which is lined by L773, W671, P672, L673, Y760, M670, F669, and H930. The shape and size of this cavity is permissive for EU1180-438 occupancy, suggesting that it could harbor the binding pocket for EU1180-438 in GluN3A.

Taken together, we identified EU1180-438 as a novel modulator for GluN1/GluN3 receptors that shows remarkable selectivity for GluN1/GluN3 receptors over all other glutamate receptors tested. The inhibitor EU1180-438 acts in a voltage-independent and non-competitive fashion. Given the specificity for GluN1/GluN3, EU1180-438 interacts primarily with GluN3 residues rather than GluN1 residues and the pre-M1 region in GluN3 is a likely target of action.

This region connects the agonist binding domain to the first M1 transmembrane domain, near a short two turn pre-M1 helix that lies parallel to the plane of the membrane.

### Example 8. EU1180-438 is Brain-Penetrable

EU1180-438 was tested in male C57BL/6 mice for brain penetrability. After a single intraperitoneal dose of 20 mg/kg the concentration of EU1180-438 in the plasma was measured. The max concentration was 2.75 μM, with a half-life of 1.57 hours. The concentration of EU1180-438 in the brain was also measured to determine if the compound can pass the blood-brain barrier. The compound was detected in the brain tissue up to 8 hours after dosage administration.

TABLE 2
Brain-to-plasma ratio of EU1180-438 in mice

| | Plasma | Brain | Brain to | |
|---|---|---|---|---|
| Time | Concentration | Concentration | Plasma | Mean |
| (hour) | (μM) | (μM) | Ratio | Ratio |
| 0.25 | 2.30 | 3.68 | 1.60 | 1.74 |
| | 2.05 | 4.06 | 1.98 | |
| | 2.21 | 3.63 | 1.64 | |
| 1 | 1.25 | 1.96 | 1.57 | 1.79 |
| | 1.04 | 2.04 | 1.96 | |
| | 1.17 | 2.14 | 1.83 | |
| 2 | 0.89 | 1.05 | 1.19 | 0.88 |
| | 0.92 | 0.66 | 0.72 | |
| | 0.96 | 0.69 | 1.72 | |
| 8 | 0.05 | 0.06 | 1.37 | 1.02 |
| | 0.04 | 0.04 | 0.90 | |
| | 0.04 | 0.03 | 0.72 | |

### Example 9. Synthesis and Characterization of 2-(1-(4-isopropoxyphenyl)-2-(4-methoxyphenoxy)ethyl)isoindolin-1-one (EU1180-481)

### A. Synthesis of methyl 2-amino-2-(4-hydroxyphenyl)acetate

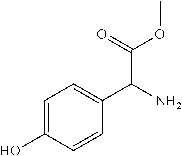

To a solution of 4-hydroxyphenylglycine (1 g, 5.98 mmol) in methanol (10 mL) was added thionyl chloride (0.52 mL, 7.18 mmol) dropwise. The reaction stirred at room temperature for 6 hours or until complete by TLC or LC-MS. Upon completion, the solvent was removed in vacuo to yield methyl-2-amino-2-(4-hydroxyphenyl)acetate (1 g, 5.52 mmol, 92.3% yield) as a pale-yellow solid. The product was confirmed via LC-MS and carried forward without further purification.

### B. Synthesis of methyl-2-(4-hydroxyphenyl)-2-(1-oxoisoindolin-2-yl)acetate

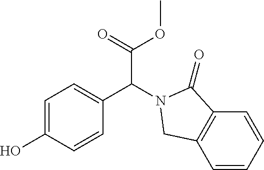 [SMILES: COC(=O)C(c1ccc(O)cc1)N1Cc2ccccc2C1=O]

To a solution of methyl 2-(bromomethyl)benzoate (250.32 mg, 1.09 mmol) in ethanol (2 mL) and triethylamine (0.28 mL, 1.99 mmol) was added methyl-2-amino-2-(4-hydroxyphenyl)acetate (180 mg, 0.99 mmol). The reaction stirred at room temperature overnight or until complete by TLC or LC-MS. Upon completion, the reaction was diluted with water and extracted three times with dichloromethane. The combined organic layers were washed once with brine, dried over anhydrous sodium sulfate and concentrated in vacuo. The residue was then purified via silica gel column chromatography using hexanes and ethyl acetate to yield methyl-2-(4-hydroxyphenyl)-2-(1-oxoisoindolin-2-yl)acetate (60.6 mg, 0.204 mmol, 20.5% yield) as a pale-yellow oil.

### C. Synthesis of methyl-2-(4-isopropoxyphenyl)-2-(1-oxoisoindolin-2-yl)acetate

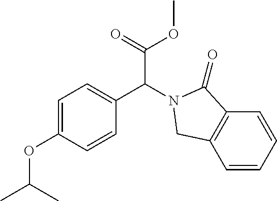 [SMILES: COC(=O)C(c1ccc(OC(C)C)cc1)N1Cc2ccccc2C1=O]

To a solution of methyl-2-(4-hydroxyphenyl)-2-(1-oxoisoindolin-2-yl)acetate (60.6 mg, 0.200 mmol) in DMF (5 mL) was added potassium carbonate (33.8 mg, 0.240 mmol) and 2-iodopropane (0.02 mL, 0.240 mmol). The reaction stirred at 50° C. under argon overnight or until complete by TLC or LC-MS. Upon completion, the reaction was diluted with water and extracted three times with ethyl acetate. The combined organic layers were washed once with brine, dried over anhydrous sodium sulfate and concentrated in vacuo. The residue was purified via silica gel column chromatography using hexanes and ethyl acetate to yield methyl-2-(4-isopropoxyphenyl)-2-(1-oxoisoindolin-2-yl)acetate (64.5 mg, 0.190 mmol, 93.2% yield) as a pale-yellow oil.

### D. Synthesis of 2-(2-hydroxy-1-(4-isopropoxyphenyl)ethyl)isoindolin-1-one

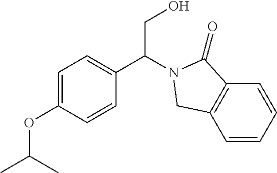 [SMILES: CC(C)Oc1ccc(C(CO)N2Cc3ccccc3C2=O)cc1]

To a solution of methyl-2-(4-isopropoxyphenyl)-2-(1-oxoisoindolin-2-yl)acetate (64.5 mg, 0.190 mmol) in tetrahydrofuran (2 mL) was added lithium borohydride (0.29 mL, 0.570 mmol). The reaction stirred at room temperature overnight until complete by TLC or LC-MS. Upon completion, the reaction was diluted with saturated ammonium chloride and extracted three times with ethyl acetate. The combined organic layers were washed once with brine, dried over anhydrous sodium sulfate and concentrated in vacuo. The product was confirmed by LC-MS and carried forward without further purification.

### E. Synthesis of 2-(1-(4-isopropoxyphenyl)-2-(4-methoxyphenoxy)ethyl)isoindolin-1-one

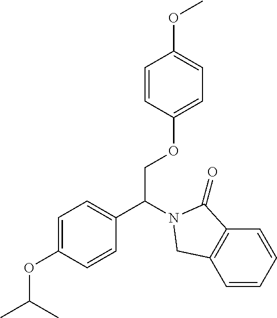 [SMILES: COc1ccc(OCC(c2ccc(OC(C)C)cc2)N2Cc3ccccc3C2=O)cc1]

An oven dried microwave vial was charged with an oven dried stir bar, 2-(2-hydroxy-1-(4-isopropoxyphenyl)ethyl)isoindolin-1-one (35 mg, 0.110 mmol), copper(I) iodide (4.28 mg, 0.0200 mmol), 3,4,7,8-tetramethyl-1,10-phenanthroline (5.31 mg, 0.0200 mmol), 1-iodo-4-methoxybenzene (39.46 mg, 0.170 mmol), and cesium carbonate (54.94 mg, 0.170 mmol). The vessel was then capped and evacuated and backfilled with argon three times. Dry, degassed, toluene (0.353 mL) was added and the vial was evacuated and backfilled once more, before heating and stirring at 110° C. for 48 hours. Once the reaction was complete, the mixture was diluted with ethyl acetate and filtered through a plug of celite, and further washed with additional ethyl acetate. The crude material was concentrated and purified via flash chromatography using hexanes and ethyl acetate to yield 2-(1-(4-isopropoxyphenyl)-2-(4-methoxyphenoxy)ethyl)isoindolin-1-one (8.8 mg, 0.0211 mmol, 18.75% yield) as a bright-yellow oil. ¹H NMR (600 MHz, CDCl₃) δ 7.86 (dt, J=1.1, 7.5 Hz, 1H), 7.48 (tt, J=1.1, 7.5 Hz, 1H), 7.43 (td, J=1.0, 7.4 Hz, 1H), 7.35 (dq, J=0.9, 7.5 Hz, 1H), 7.33-7.29 (m, 2H), 6.85-6.81 (m, 4H), 6.81-6.77 (m, 2H), 5.83 (t, J=5.8 Hz, 1H), 4.57-4.48 (m, 3H), 4.44 (ddd, J=0.9, 5.1, 10.0 Hz, 1H), 4.22 (d, J=16.9 Hz, 1H), 3.73 (d, J=0.9 Hz, 3H), 1.30 (dd, J=4.3, 6.0 Hz, 6H). ¹³C NMR (151 MHz, CDCl₃) δ 196.89, 172.47, 168.64, 157.53, 152.41, 141.68, 132.58, 131.27, 129.09, 128.98, 127.88, 123.79, 122.68, 115.88, 115.75, 114.65, 69.81, 68.70, 55.68, 53.26, 47.76, 21.99, 21.97. HRMS calcd. for C₂₆H₂₈O₄N [M+H]⁺ 418.20128; found 418.20106. Purity was established using an Agilent pump on a Zorbax XBD-C18 column (4.6 mm×50 mm, 3.5 μm). Method 1: 75-95% MeOH in water over 6 min at 1 mL/min (retention time=2.59 min). Method 2: 50-95% MeOH in water over 6 min at 1 mL/min (retention time 5.63 min).

### Example 10. Synthesis and Characterization of 2-(2-(4-methoxyphenoxy)-1-(4-methoxyphenyl)ethyl)-3,4-dihydroisoquinolin-1(2H)-one (EU1180-489)

### A. Synthesis of methyl-2-hydroxy-2-(4-methoxyphenyl)acetate

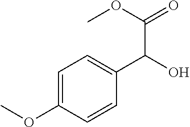

To a solution of 2-hydroxy-2-(4-methoxyphenyl)acetic acid (6.24 g, 34.25 mmol) in methanol (20 mL), thionyl chloride (3.25 mL, 44.53 mmol) was added dropwise. The reaction stirred at room temperature until complete by TLC or LC-MS. Upon completion, the solvent was removed in vacuo, washed once with methanol and evaporated to dryness. The residue was purified via silica gel column chromatography using hexanes and ethyl acetate to yield methyl-2-hydroxy-2-(4-methoxyphenyl)acetate (4.75 g, 24.21 mmol, 70.68% yield) as a white solid.

### B. Synthesis of methyl-2-(4-methoxyphenyl)-2-(1-oxo-3,4-dihydroisoquinolin-2-yl)acetate

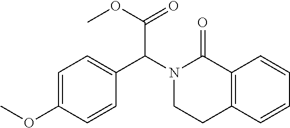 [SMILES: COC(=O)C(c1ccc(OC)cc1)N1CCc2ccccc2C1=O]

Step 1: In a flame dried flask under argon containing a solution of methyl-2-hydroxy-2-(4-methoxyphenyl)acetate (2.95 g, 15.04 mmol) in anhydrous dichloromethane (50.12 mL) was added phosphorous tribromide (0.99 mL, 10.52 mmol) at 0° C. The reaction stirred at 0° C. for one hour. Afterwards, the reaction was diluted with water and extracted three times with dichloromethane. The combined organic layers were washed with brine, dried over anhydrous sodium sulfate and concentrated in vacuo. The residue was taken up in 5 mL anhydrous tetrahydrofuran and added to the second step.

Step 2: 3,4-Dihydro-2H-isoquinolin-1-one (2.21 g, 15.04 mmol) in 30 mL anhydrous tetrahydrofuran at 0° C. was added sodium hydride (60% in mineral oil)(721.71 mg, 18.04 mmol). After stirring for 30 minutes, the brominated product from Step 1 was added slowly. The reaction continued to stir at room temperature overnight or until complete by TLC or LC-MS. Upon completion, the reaction was diluted with aqueous ammonium chloride and extracted three times with ethyl acetate. The combined organic layers were washed once with brine, dried over anhydrous sodium sulfate and concentrated in vacuo. The residue was purified via silica gel column chromatography using hexanes and ethyl acetate to yield methyl-2-(4-methoxyphenyl)-2-(1-oxo-3,4-dihydroisoquinolin-2-yl)acetate (2.74 g, 8.42 mmol, 56.01% yield) as a pale-yellow solid.

### C. Synthesis of 2-(2-hydroxy-1-(4-methoxyphenyl)ethyl)-3,4-dihydroisoquinolin-1-one

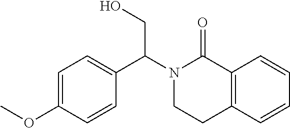 [SMILES: COc1ccc(C(CO)N2CCc3ccccc3C2=O)cc1]

To a solution of methyl-2-(4-methoxyphenyl)-2-(1-oxo-3,4-dihydroisoquinolin-2-yl)acetate (2.74 g, 8.42 mmol) in tetrahydrofuran (84 mL) at 0° C., lithium borohydride (16.84 mL, 33.69 mmol) was slowly added. The reaction mixture stirred at room temperature for 2-5 hours or until complete by TLC or LC-MS. Upon completion, the reaction was diluted with aqueous ammonium chloride and extracted three times with ethyl acetate. The combined organic layers were washed once with brine, dried over anhydrous sodium sulfate and concentrated in vacuo. The residue was purified via silica gel column chromatography using hexanes and ethyl acetate to yield 2-(2-hydroxy-1-(4-methoxyphenyl)ethyl)-3,4-dihydroisoquinolin-1-one (1.93 g, 6.49 mmol, 77.03% yield) as a white sticky foam.

### D. Synthesis of 2-(2-(4-methoxyphenoxy)-1-(4-methoxyphenyl)ethyl)-3,4-dihydroisoquinolin-1(2H)-one

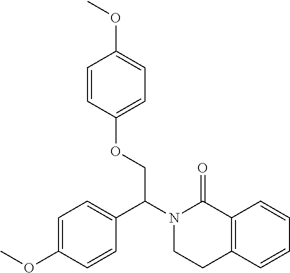 [SMILES: COc1ccc(OCC(c2ccc(OC)cc2)N2CCc3ccccc3C2=O)cc1]

To an oven-dried microwave tube with a stir bar, 2-(2-hydroxy-1-(4-methoxyphenyl)ethyl)-3,4-dihydroisoquinolin-1-one (0.3 g, 1.03 mmol), copper(I) iodide (8.14 mg, 0.0400 mmol), cesium carbonate (417.66 mg, 1.28 mmol), 3,4,7,8-tetramethyl-1,10-phenanthroline (20.19 mg, 0.0900 mmol), and 1-iodo-4-methoxybenzene (200 mg, 0.850 mmol) were added and then covered with a rubber septum. The tube was evacuated with vacuum and back-filled with argon three times. Degassed toluene (2 mL) was added and the tube was capped quickly. The tube was heated at 110° C. for 48 hours. Upon completion, the reaction was filtered through a pad of silica gel, and eluted with excess ethyl acetate. The filtrate was concentrated and purified via silica gel column chromatography using hexanes and ethyl acetate to yield 2-(2-(4-methoxyphenoxy)-1-(4-methoxyphenyl)ethyl)-3,4-dihydroisoquinolin-1(2H)-one (257 mg, 0.637 mmol, 74.54% yield). ¹H NMR (600 MHz, CDCl₃) δ 8.12 (dd, J=1.4, 7.7 Hz, 1H), 7.42-7.30 (m, 4H), 7.13-7.09 (m, 1H), 6.91-6.84 (m, 4H), 6.84-6.77 (m, 2H), 6.27 (t, J=5.7 Hz, 1H), 4.49-4.41 (m, 2H), 3.79 (s, 3H), 3.74 (s, 3H), 3.54 (ddd, J=5.1, 8.5, 12.4 Hz, 1H), 3.31 (ddd, J=5.2, 7.2, 12.4 Hz, 1H), 2.91-2.76 (m, 2H). ¹³C NMR (151 MHz, CDCl₃) δ 164.80, 159.07, 154.17, 152.69, 138.28, 131.71, 129.68, 129.42, 129.29, 128.55, 127.02, 126.82, 115.74, 114.71, 114.02, 68.00, 55.74, 55.28, 54.38, 42.37, 28.45. HRMS calcd. for C₂₅H₂₆NO₄ [M+H]⁺404.18563; found 404.18608. Purity was determined by LC-MS using an Agilent pump on a Zorbax XBD-C18 column (4.6 mm×50 mm, 3.5 μm). Method 1: 85-95% MeOH in water over 5 min at 1 mL/min (retention time=1.44 min). Method 2: 75-95% MeOH in water over 3 min at 1 mL/min (retention time 2.07 min).

### Example 11. Synthesis and Characterization of 2-(2-(4-methoxyphenoxy)-1-phenylethyl)-3,4-dihydroisoquinolin-1(2H)-one (EU1180-488)

### A. Synthesis of methyl-2-bromo-2-phenyl-acetate

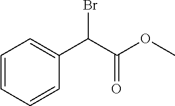

To a solution of methyl-2-phenylacetate (5 g, 33.3 mmol) in carbon tetrachloride (20 mL) was added N-bromosuccinimide (7.11 g, 39.95 mmol) followed by 2,2′-azobis(2-methylpropionitrile) (0.05 g, 0.330 mmol). The reaction mixture stirred at reflux until complete by TLC or LC-MS, approximately 4 hours. Upon completion, the reaction was diluted with hexanes and filtered through a plug of silica. The filtrate was concentrated in vacuo to yield methyl-2-bromo-2-phenyl-acetate (6.8 g, 29.685 mmol, 89.16% yield).

### B. Synthesis of methyl-2-(1-oxo-3,4-dihydroisoquinolin-2-yl)-2-phenyl-acetate

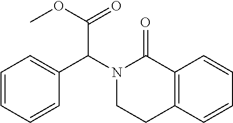 [SMILES: COC(=O)C(c1ccccc1)N1CCc2ccccc2C1=O]

To a solution of 3,4-dihydro-2H-isoquinolin-1-one (209 mg, 1.42 mmol) in DMF (10 mL) was added sodium hydride (60% in mineral oil) (62.49 mg, 1.56 mmol). The mixture stirred for 30 minutes before the addition of methyl-2-bromo-2-phenyl-acetate (357.84 mg, 1.56 mmol). The reaction stirred at room temperature until complete by TLC or LC-MS. Upon completion, the reaction was quenched with an aqueous solution of ammonium chloride. This mixture was extracted three times with ethyl acetate. The combined organic layers were washed once with brine, dried over anhydrous sodium sulfate and concentrated in vacuo. The resulting residue was purified via silica gel column chromatography using hexanes and ethyl acetate to yield methyl-2-(1-oxo-3,4-dihydroisoquinolin-2-yl)-2-phenyl-acetate (78 mg, 0.264 mmol, 18.60% yield) as a colorless oil.

### C. Synthesis of 2-(2-hydroxy-1-phenylethyl)-3,4-dihydroisoquinolin-1(2H)-one

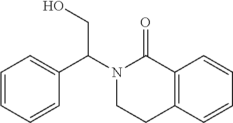 [SMILES: O=C1c2ccccc2CCN1C(CO)c1ccccc1]

To a solution of methyl-2-(1-oxo-3,4-dihydroisoquinolin-2-yl)-2-phenyl-acetate (965 mg, 3.27 mmol) in tetrahydrofuran (33 mL) at 0° C. was added lithium borohydride (6.54 mL, 13.07 mmol) (2 M in tetrahydrofuran). The reaction stirred at room temperature overnight or until complete by TLC or LC-MS. Upon completion, the reaction was diluted with aqueous ammonium chloride and extracted three times with ethyl acetate. The combined organic layers were washed once with brine, dried over anhydrous sodium sulfate and concentrated in vacuo. The residue was purified via silica gel column chromatography to yield 2-(2-hydroxy-1-phenylethyl)-3,4-dihydroisoquinolin-1(2H)-one (965 mg, 3.27 mmol, 85%) as a white foam.

### D. Synthesis of 2-(2-(4-methoxyphenoxy)-1-phenylethyl)-3,4-dihydroisoquinolin-1(2H)-one

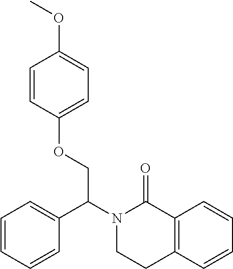 [SMILES: COc1ccc(OCC(c2ccccc2)N2CCc3ccccc3C2=O)cc1]

An oven-dried microwave tube was equipped with 2-(2-hydroxy-1-phenylethyl)-3,4-dihydroisoquinolin-1(2H)-one (137.07 mg, 0.510 mmol), 1-iodo-4-methoxybenzene (80 mg, 0.340 mmol), copper(I) iodide (3.26 mg, 0.020 mmol), cesium carbonate (167.07 mg, 0.510 mmol), and 3,4,7,8-tetramethyl-1,10-phenanthroline (8.08 mg, 0.030 mmol). The tube was fitted with a rubber septum, evacuated and backfilled with argon three times. Degassed toluene (0.684 mL) was added, the septum was removed, and the tube was capped and sealed quickly. The tube was heated in a heat-block to 110° C. for 24-48 hours until the reaction was shown to be complete by TLC or LC-MS. Upon completion, the cooled reaction was filtered through a pad of silica gel, eluted with excess ethyl acetate. The filtrate was concentrated and purified via silica gel column chromatography using hexanes and ethyl acetate to yield 2-(2-(4-methoxyphenoxy)-1-phenylethyl)-3,4-dihydroisoquinolin-1(2H)-one (51.6 mg, 0.138 mmol, 40.42% yield). ¹H NMR (600 MHz, CDCl₃) δ 8.14 (dd, J=1.5, 7.8 Hz, 1H), 7.47-7.43 (m, 2H), 7.40 (td, J=1.5, 7.5 Hz, 1H), 7.37-7.32 (m, 4H), 7.32-7.26 (m, 1H), 7.13 (dd, J=1.2, 7.5 Hz, 1H), 6.90-6.85 (m, 2H), 6.85-6.79 (m, 2H), 6.34 (t, J=5.7 Hz, 1H), 4.50 (dd, J=2.5, 5.7 Hz, 2H), 3.74 (s, 2H), 3.57 (ddd, J=5.5, 8.2, 12.5 Hz, 1H), 3.33 (ddd, J=5.5, 6.8, 12.4 Hz, 1H), 2.88-2.82 (m, 2H). ¹³C NMR (151 MHz, CDCl₃) δ 164.89, 154.22, 152.67, 138.31, 137.48, 129.63, 128.69, 128.60, 128.01, 127.75, 127.05, 126.86, 115.76, 114.73, 114.26, 67.87, 55.75, 54.93, 42.57, 28.44. HRMS calc. for C₂₄H₂₄NO₃ [M+H]⁺374.17507; found 374.17540. Purity was determined by LC-MS using an Agilent pump on a Zorbax XBD-C18 column (4.6 mm×50 mm, 3.5 μm). Method 1: 50-95% MeOH in water over 6 min at 1 mL/min (retention time=5.40 min). Method 2: 75-95% MeOH in water over 3 min at 1 mL/min (retention time 2.17 min).

### Example 12. Synthesis and Characterization of 2-(2-(4-methoxyphenoxy)-1-phenyl-ethyl)isoindolin-1-one (EU1180-487)

### A. Synthesis of methyl-2-(1-oxoisoindolin-2-yl)-2-phenyl-acetate

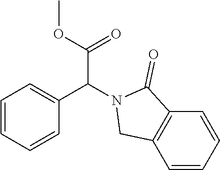 [SMILES: COC(=O)C(c1ccccc1)N1Cc2ccccc2C1=O]

To a solution of isoindolin-1-one (100 mg, 0.750 mmol) in dimethylformamide (3 mL), sodium hydride (60% in mineral oil) (33.05 mg, 0.830 mmol) was added at room temperature.

After stirring for 30 minutes, methyl-2-bromo-2-phenyl-acetate (189.24 mg, 0.830 mmol) was added dropwise. The reaction continued to stir at room temperature overnight or until complete by TLC or LC-MS. Upon completion, the reaction was cooled to 0° C., quenched with saturated aqueous ammonium chloride and extracted three times with ethyl acetate. The combined organic layers were washed once with brine, dried over anhydrous sodium sulfate and concentrated in vacuo to yield methyl-2-(1-oxoisoindolin-2-yl)-2-phenyl-acetate (98.7 mg, 0.351 mmol, 46.72% yield) as a thick pale-yellow oil. The crude material was carried forward without further purification.

B. Synthesis of 2-(2-hydroxy-1-phenyl-ethyl)isoindolin-1-one

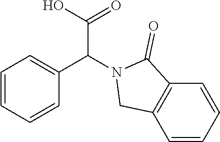 [SMILES: O=C(O)C(c1ccccc1)N1Cc2ccccc2C1=O]

To a solution of methyl-2-(1-oxoisoindolin-2-yl)-2-phenyl-acetate (537 mg, 1.91 mmol) in tetrahydrofuran (19 mL) at 0° C. lithium borohydride (3.82 mL, 7.64 mmol) (2M in tetrahydrofuran) were added slowly. The reaction stirred at room temperature overnight or until complete by TLC or LC-MS. Upon completion, the reaction was diluted with aqueous ammonium chloride and extracted three times with ethyl acetate. The combined organic layers were washed once with brine, dried over anhydrous sodium sulfate and concentrated in vacuo. The residue was purified via silica gel column chromatography using hexanes and ethyl acetate to obtain 2-(2-hydroxy-1-phenyl-ethyl)isoindolin-1-one (347 mg, 1.370 mmol, 71.76% yield) as a light-yellow foam.

C. Synthesis of 2-(2-(4-methoxyphenoxy)-1-phenyl-ethyl)isoindolin-1-one

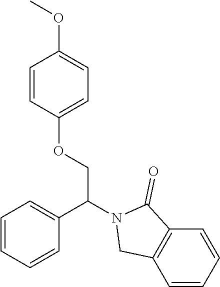 [SMILES: COc1ccc(OCC(c2ccccc2)N2Cc3ccccc3C2=O)cc1]

In an oven-dried microwave vial equipped with a stir-bar, copper(I) iodide (7.07 mg, 0.0400 mmol), 3,4,7,8-tetramethyl-1,10-phenanthroline (8.77 mg, 0.0400 mmol), cesium carbonate (90.68 mg, 0.280 mmol), 1-iodo-4-methoxybenzene (65.14 mg, 0.280 mmol), and 2-(2-hydroxy-1-phenyl-ethyl)isoindolin-1-one (47 mg, 0.190 mmol) were added. The tube was sealed; purged and back-filled with argon three-times. Degassed toluene (0.5 mL) was added and the reaction stirred at 110° C. for 48 hours until complete by TLC or LC-MS. Upon completion, the reaction was cooled to room temperature, filtered through a pad of silica gel, and eluted with ethyl acetate. The filtrate was concentrated, and the resulting residue was purified via silica gel column chromatography using hexanes and ethyl acetate to yield 2-(2-(4-methoxyphenoxy)-1-phenyl-ethyl)isoindolin-1-one (17 mg, 0.0473 mmol, 25.49% yield). ¹H NMR (600 MHz, CDCl₃) δ 7.93-7.84 (m, 1H), 7.53-7.25 (m, 8H), 6.90-6.84 (m, 2H), 6.82-6.78 (m, 2H), 5.90 (dd, J=5.0, 6.5 Hz, 1H), 4.67-4.44 (m, 3H), 4.24 (d, J=16.9 Hz, 2H), 3.76-3.70 (m, 3H). ¹³C NMR (151 MHz, CDCl₃) δ 172.47, 168.74, 152.35, 149.39, 141.67, 137.24, 131.36, 128.80, 127.95, 127.93, 127.80, 123.84, 122.72, 115.75, 114.67, 68.56, 55.70, 53.81, 47.85. HRMS calcd. for C₂₃H₂₂NO₃ [M+H]⁺ 360.15942; found 360.15892. Purity was determined by LC-MS using an Agilent pump on a Zorbax XBD-C18 column (4.6 mm×50 mm, 3.5 μm). Method 1: 75% MeOH in water over 3 min at 1 mL/min (retention time=1.73 min). Method 2: 75-95% MeOH in water over 3 min at 1 mL/min (retention time 1.71 min).

### Example 13. Synthesis and Characterization of 2-(1-(3,4-dimethoxyphenyl)-2-(4-methoxyphenoxy)ethyl)isoindolin-1-one (EU1180-494)

### A. Synthesis of ethyl-2-(3,4-dimethoxyphenyl)-2-oxo-acetate

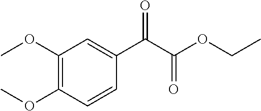 [SMILES: CCOC(=O)C(=O)c1ccc(OC)c(OC)c1]

In a round-bottom flask, in a brine and ice bath, under an argon atmosphere, aluminum chloride (1.74 g, 13.03 mmol) was suspended in dichloromethane (15 mL). To this mixture, ethyl oxalyl chloride (1.46 mL, 13.03 mmol) was added dropwise. After approximately 10 minutes, 1,2-dimethoxybenzene (1 g, 7.24 mmol) was added dropwise. After which, the reaction stirred at room temperature for one hour. Once complete, the reaction was diluted slowly with water and extracted three times with ethyl acetate. The combined organic layers are washed once with brine, dried over anhydrous sodium sulfate and concentrated in vacuo. The residue was purified via silica gel column chromatography to yield ethyl-2-(3,4-dimethoxyphenyl)-2-oxo-acetate (5.17 g, 21.70 mmol, 99.94% yield) as a colorless oil.

### B. Synthesis of ethyl-2-(3,4-dimethoxyphenyl)-2-hydroxy-acetate

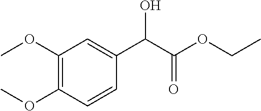 [SMILES: CCOC(=O)C(O)c1ccc(OC)c(OC)c1]

Under inert conditions, ethyl-2-(3,4-dimethoxyphenyl)-2-oxo-acetate (1.7 g, 7.14 mmol) was suspended in ethanol (50 mL) in a dry-ice acetonitrile bath. Sodium borohydride (0.27 g, 7.14 mmol) was added in small portions. The reaction was checked via LC-MS after 15 minutes. Upon completion, the reaction was quenched by the addition of 10% hydrochloric acid. The reaction was then diluted with deionized water and extracted three times with ethyl acetate. The combined organic layers were washed with brine, dried over anhydrous sodium sulfate and concentrated in vacuo. The residue was purified via silica gel column chromatography using hexanes and ethyl acetate to yield ethyl-2-(3,4-dimethoxyphenyl)-2-hydroxy-acetate (877 mg, 3.65 mmol, 51.16% yield) as a colorless oil.

### C. Synthesis of ethyl-2-(3,4-dimethoxyphenyl)-2-(1-oxoisoindolin-2-yl)acetate

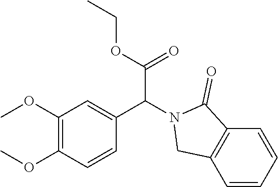 [SMILES: CCOC(=O)C(c1ccc(OC)c(OC)c1)N1Cc2ccccc2C1=O]

Step 1: To a solution of ethyl-2-(3,4-dimethoxyphenyl)-2-hydroxy-acetate (530 mg, 2.21 mmol) in dichloromethane (10 mL) at 0° C., phosphorous tribromide (0.14 mL, 1.46 mmol) was added. The reaction stirred at this temperature for 2 hours or until complete by TLC. The reaction mixture was diluted with deionized water and extracted three times with dichloromethane. The combined organic layers were washed once with brine, dried over anhydrous sodium sulfate, and concentrated in vacuo. The residue was taken up in tetrahydrofuran (5 mL) and added dropwise to the second step.

Step 2: To a solution of isoindolin-1-one (323.11 mg, 2.43 mmol) in tetrahydrofuran (10 mL), sodium hydride (60% in mineral oil) (105.89 mg, 2.65 mmol) was added. After stirring at room temperature for 30 minutes, the brominated product from Step 1 was added. The reaction mixture continued to stir at room temperature overnight or until complete by TLC or LC-MS. Upon completion, the reaction was diluted with aqueous ammonium chloride and extracted three times with ethyl acetate. The combined organic layers were washed once with brine, dried over anhydrous sodium sulfate and concentrated in vacuo. The crude material was purified via silica gel column chromatography using hexanes and ethyl acetate to yield ethyl-2-(3,4-dimethoxyphenyl)-2-(1-oxoisoindolin-2-yl)acetate (302.9 mg, 0.852 mmol, 38.64% yield) as a yellow solid.

### D. Synthesis of 2-(1-(3,4-dimethoxyphenyl)-2-hydroxy-ethyl)isoindolin-1-one

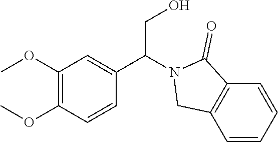 [SMILES: COc1ccc(C(CO)N2Cc3ccccc3C2=O)cc1OC]

To a solution of ethyl-2-(3,4-dimethoxyphenyl)-2-(1-oxoisoindolin-2-yl)acetate (783.90 mg, 2.21 mmol) in ethanol (20 mL) at room temperature, sodium borohydride (417.23 mg, 11.03 mmol) was added portion-wise over a two-hour period. The reaction continued to stir at room temperature overnight until complete by TLC or LC-MS. Upon completion, the reaction was slowly quenched with water and extracted three times with dichloromethane. The combined organic layers were washed once with brine, dried over anhydrous sodium sulfate and concentrated in vacuo. The crude material was used without further purification.

### E. Synthesis of 2-(1-(3,4-dimethoxyphenyl)-2-(4-methoxyphenoxy)ethyl)isoindolin-1-one

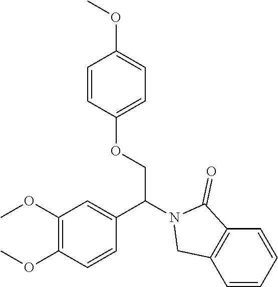 [SMILES: COc1ccc(OCC(c2ccc(OC)c(OC)c2)N2Cc3ccccc3C2=O)cc1]

To an oven-dried microwave tube containing a stir-bar, cesium carbonate (120.08 mg, 0.370 mmol), copper(I) iodide (2.34 mg, 0.010 mmol), 1-iodo-4-methoxybenzene (57.5 mg, 0.250 mmol), and 3,4,7,8-tetramethyl-1,10-phenanthroline (5.81 mg, 0.020 mmol) were added. The tube was purged via vacuum and filled with argon three times. 2-(1-(3,4-dimethoxyphenyl)-2-hydroxy-ethyl)isoindolin-1-one (92.39 mg, 0.290 mmol) was dissolved in degassed toluene (1 mL) and added to the reaction mixture and the tube was capped quickly. The reaction was heated at 110° C. for 48 hours until complete by TLC or LC-MS. Upon completion, the reaction was filtered through celite and eluted with ethyl acetate. The filtrate was concentrated in vacuo and purified via silica gel column chromatography to afford 2-(1-(3,4-dimethoxyphenyl)-2-(4-methoxyphenoxy)ethyl)isoindolin-1-one (29 mg, 0.0691 mmol, 28.14% yield) as an amber-oil. ¹H NMR (500 MHz, CDCl₃) δ 7.89 (ddd, J=0.8, 1.3, 7.5 Hz, 1H), 7.54-7.49 (m, 1H), 7.49-7.44 (m, 1H), 7.41-7.36 (m, 1H), 7.02 (ddd, J=0.7, 2.1, 8.2 Hz, 1H), 6.98 (d, J=2.1 Hz, 1H), 6.92-6.79 (m, 6H), 5.86 (t, J=5.8 Hz, 1H), 4.63-4.52 (m, 2H), 4.53-4.44 (m, 1H), 4.24 (d, J=17.0 Hz, 1H), 3.87 (s, 3H), 3.84 (s, 3H), 3.76 (s, 4H). ¹³C NMR (126 MHz, CDCl₃) δ 168.65, 154.30, 152.43, 149.24, 148.81, 141.69, 132.56, 131.35, 129.88, 129.12, 127.93, 123.82, 122.72, 119.87, 115.84, 114.73, 114.15, 111.72, 111.15, 68.84, 56.00, 55.91, 55.71, 53.62, 47.87. HRMS calcd. for C₂₅H₂₆NO₅ [M+H]⁺420.1806; found 420.1802. Purity was determined by LC-MS using an Agilent pump on a Zorbax XBD-C18 column (4.6 mm×50 mm, 3.5 μm). Method 1: 50-95% MeOH in water over 3 min at 1 mL/min (retention time=1.81 min). Method 2: 75-95% MeOH in water over 3 min at 1 mL/min (retention time 0.94 min).

### Example 14. Biological Activity of EU1180-481, EU1180-487, EU1180-488, EU1180-489, and

EU1180-494

EU1180-481, EU1180-487, EU1180-488, EU1180-489, and EU1180-494 were screened in the same assay as EU1180-438 to determine their effect on hGluN1^{FA,TL}/hGluN3A receptors. Compounds that inhibited by more than 20% at 10 μM were analyzed further, and a dose-response curve was generated, as shown in FIG. 5. Table 3 summarizes the biological activity results of EU1180-481, EU1180-487, EU1180-488, EU1180-489, and EU1180-494. These compounds were also shown to have no detectable activity (at 30 μM) toward recombinant GluN1/GluN2 NMDA receptors (including those containing GluN2A, GluN2B, GluN2C, or GluN2D).

TABLE 3
IC₅₀ and maximum inhibition values of EU1180-438 derivatives ^{a}

| | |
|---|---|
| IC₅₀ (95% CI) | |
| GluN3A | Max Inhibition |

| | | | |
|---|---|---|---|
| EU1180-481 | 3.12 | (2.31, 3.54) | 54% |
| EU1180-487 | 10.75 | (9.64, 11.69) | 78% |
| EU1180-488 | 3.29 | (2.94, 3.57) | 88% |
| EU1180-489 | 10.82 | (9.73, 11.86) | 44% |

| | | |
|---|---|---|
| EU1180-494 | NE | 3% |

^{a} Data are shown as mean with 95% confidence interval given in parentheses, determined from log(IC₅₀). Percentage inhibition was determined from fitted maximum at saturating test compound calculated as 100 × (1 − I_{drug}/I_{control}). Max potentiation is reported as percent potentiation at 10 μM drug. Data are from between 6 and 19 oocytes from 2-3 frogs for each compound.

### Example 15. Synthesis and Characterization of 2-(1-(4-isopropoxyphenyl)-2-((6-methylpyridin-2-yl)oxy)ethyl)isoindolin-1-one (EU1180-508)

EU1180-508 was synthesized according to the steps shown in Scheme 2 below.

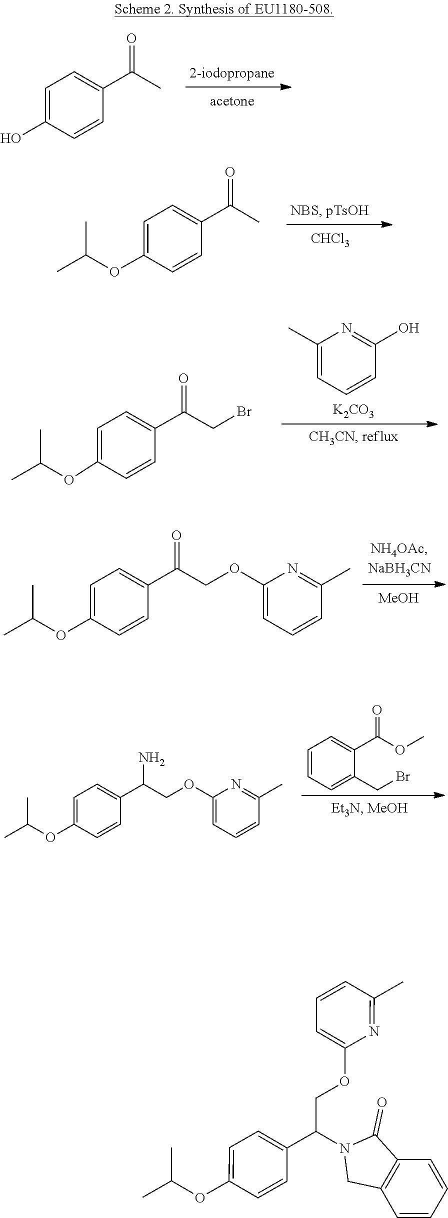

### A. Synthesis of 4-isopropoxyacetophenone

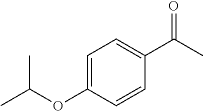

4-hydroxyacetophenone (1 eq., 73.5 mmol, 10.0 g) was dissolved in acetone (150 mL), and 2-iodopropane (2 eq., 147 mmol, 14.7 mL) and anhydrous potassium carbonate (2 eq., 147 mmol, 20.3 g) were added. The reaction mixture was stirred under reflux for 24 hours, after which the acetone was removed under reduced pressure, and the product was partitioned between ethyl acetate and diH₂O. The organic layer was decanted, dried over anhydrous Na₂SO₄, filtered, and concentrated to a colorless oil to afford 4-isopropoxyacetophenone that was deemed pure by NMR.

Yield: 12.96 g (72.7 mmol, 99%).

### B. Synthesis of 2-bromo-4′-isopropoxyacetophenone

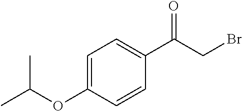

The starting 4-isopropoxyacetophenone (1 eq., 72.66 mmol, 12.95 g) was dissolved in chloroform (150 mL total). While stirring at room temperature, p-toluenesulfonic acid (0.2 eq., 14.53 mmol, 2.50 g) and N-bromosuccinimide (NBS, 1.1 eq., 79.93 mmol, 14.23 g) were subsequently added, and the solution was magnetically stirred room temperature overnight. After the organic phase was washed with diH₂O, the organic layer was separated, dried over anhydrous Na₂SO₄, and concentrated to a golden-colored viscous oil. The product was purified by flash column chromatography (silica gel, 0-60% dichloromethane in hexanes) to afford 2-bromo-4′-isopropoxyacetophenone as a colorless oil. Yield: 17.15 g (66.7 mmol, 92%).

### C. Synthesis of 1-(4-isopropoxyphenyl)-2-((6-methylpyridin-2-yl)oxy)ethan-1-one

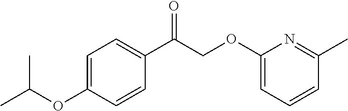 [SMILES: Cc1cccc(OCC(=O)c2ccc(OC(C)C)cc2)n1]

A solution of 2-bromo-4′-isopropoxyacetophenone (1 eq., 9.72 mmol, 2.50 g) in acetonitrile (50 mL) was supplemented with crushed anhydrous potassium carbonate (1.5 eq., 14.58 mmol, 2.02 g) and 2-hydroxy-6-methylpyridine (1.2 eq., 11.67 mmol, 1.27 g). The reaction mixture was stirred under reflux for 24 hours. After removing the acetonitrile under reduced pressure, the product was reconstituted in ethyl acetate (100 mL), and the organic phase was washed with diH₂O. The organic layer was removed, dried over anhydrous Na₂SO₄, filtered, and concentrated to a light brown crude oil. The product was purified by flash column chromatography (silica gel, 0-20% ethyl acetate in hexanes) to afford the product as a yellow oil. Yield: 2.44 g (8.55 mmol, 88%).

### D. Synthesis of 1-(4-isopropoxyphenyl)-2-((6-methylpyridin-2-yl)oxy)ethan-1-amine

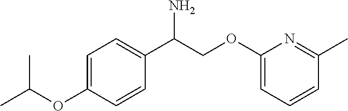 [SMILES: Cc1cccc(OCC(N)c2ccc(OC(C)C)cc2)n1]

Synthesized according to General Procedure A using 1-(4-isopropoxyphenyl)-2-((6-methylpyridin-2-yl)oxy)ethan-1-one (1 eq., 8.55 mmol, 2.44 g), NaCNBH₃ (6 eq., 51.3 mmol, 3.22 g), NH₄OAc (10 eq., 85.5 mmol, 6.59 g), and MeOH (60 mL). Yield: 1.86 g (6.50 mmol, 74%).

### E. Synthesis of 2-(1-(4-isopropoxyphenyl)-2-((6-methylpyridin-2-yl)oxy)ethyl)isoindolin-1-one

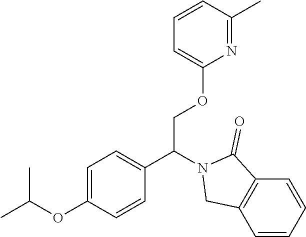 [SMILES: Cc1cccc(OCC(c2ccc(OC(C)C)cc2)N2Cc3ccccc3C2=O)n1]

Synthesized according to General Procedure C using 1-(4-isopropoxyphenyl)-2-((6-methylpyridin-2-yl)oxy)ethan-1-amine (1 eq., 0.279 mmol, 80 mg), methyl 2-(bromomethyl)benzoate (1.2 eq., 0.335 mmol, 77 mg), triethylamine (2.1 eq., 0.586 mmol, 82 μL), and MeOH (4 mL). Yield: 24 mg (0.060 mmol, 21%). ¹H NMR (500 MHz, CDCl₃) δ 1.32 (dd, J=6.1, 3.4 Hz, 6H), 2.40 (s, 3H), 4.23 (d, J=16.9 Hz, 1H), 4.48-4.57 (m, 2H), 4.85 (dd, J=11.4, 5.1 Hz, 2H), 4.98 (dd, J=11.4, 8.2 Hz, 1H), 5.93 (dd, J=8.2, 5.1 Hz, 1H), 6.50 (dt, J=8.2, 0.6 Hz, 1H), 6.67 (dt, J=7.3, 0.7 Hz, 1H), 6.86 (d, J=8.8 Hz, 2H), 7.33-7.45 (m, 5H), 7.49 (td, J=7.4, 1.2 Hz, 1H), 7.85 (td, J=7.5, 0.9 Hz, 1H). ¹³C NMR (125 MHz, CDCl₃) δ 22.2, 24.2, 47.3, 53.4, 64.8, 70.0, 107.9, 116.1, 116.2, 122.8, 123.9, 128.0, 128.1, 129.4, 131.3, 132.9, 139.0, 141.8, 156.2, 157.7, 162.8, 168.8. HRMS (APCI) m/z calculated for C₂₅H₂₇N₂O₃ [M+H]⁺403.20162, found 403.20057.

### Example 16. Synthesis and Characterization of 6-chloro-2-(1-(4-isopropoxyphenyl)-2-((6-methylpyridin-2-yl)oxy)ethyl)isoindolin-1-one (EU1180-509)

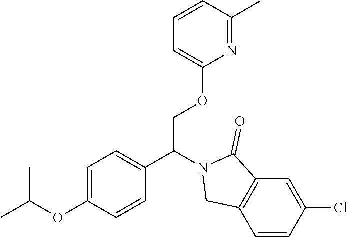 [SMILES: Cc1cccc(OCC(c2ccc(OC(C)C)cc2)N2Cc3ccc(Cl)cc3C2=O)n1]

Synthesized according to General Procedure C using 1-(4-isopropoxyphenyl)-2-((6-methylpyridin-2-yl)oxy)ethan-1-amine (1 eq., 0.384 mmol, 110 mg), methyl 2-(bromomethyl)-5-chlorobenzoate (1.2 eq., 0.461 mmol, 122 mg), triethylamine (2.1 eq., 0.806 mmol, 113 μL), and MeOH (5 mL). Yield: 30 mg (0.069 mmol, 18%). ¹H NMR (500 MHz, CDCl₃) δ 1.32 (dd, J=6.1, 3.2 Hz, 6H), 2.40 (s, 3H), 4.19 (d, J=17.1 Hz, 1H), 4.50-4.55 (m, 2H), 4.83 (dd, J=11.4, 5.0 Hz, 2H), 4.98 (dd, J=11.4, 8.3 Hz, 1H), 5.90 (dd, J=8.3, 5.0 Hz, 1H), 6.50 (d, J=8.2 Hz, 1H), 6.67 (d, J=7.2 Hz 1H), 6.87 (d, J=8.8 Hz, 2H), 7.29 (dd, J=8.1, 0.4 Hz, 1H), 7.33 (d, J=8.5 Hz, 2H), 7.40 (dd, J=8.2, 7.3 Hz, 1H), 7.44 (dd, J=8.1, 2.0 Hz, 1H), 7.80 (dd, J=2.0, 0.5 Hz, 1H).

¹³C NMR (125 MHz, CDCl₃) δ 22.1, 24.2, 47.1, 53.6, 64.6, 70.0, 107.9, 116.1, 116.3, 124.1, 129.1, 131.5, 134.3, 134.7, 139.1, 139.9, 156.1, 157.8, 162.7, 167.5. HRMS (APCI) m/z calculated for C₂₅H₂₆ClN₂O₃ [M+H]⁺437.16265, found 437.16178.

### Example 17. Synthesis and Characterization of 5-chloro-2-(1-(4-isopropoxyphenyl)-2-((6-methylpyridin-2-yl)oxy)ethyl)isoindolin-1-one (EU1180-510)

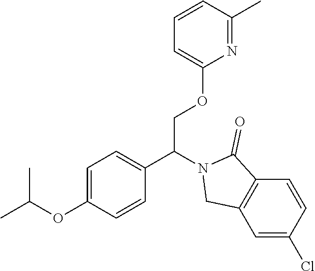 [SMILES: Cc1cccc(OCC(c2ccc(OC(C)C)cc2)N2Cc3cc(Cl)ccc3C2=O)n1]

Synthesized according to General Procedure C using 1-(4-isopropoxyphenyl)-2-((6-methylpyridin-2-yl)oxy)ethan-1-amine (1 eq., 0.419 mmol, 120 mg), methyl 2-(bromomethyl)-4-chlorobenzoate (1.2 eq., 0.503 mmol, 133 mg), triethylamine (2.1 eq., 0.880 mmol, 123 μL), and MeOH (5 mL). Yield: 41 mg (0.094 mmol, 22%). ¹H NMR (500 MHz, CDCl₃) δ 1.32 (dd, J=6.1, 3.2 Hz, 6H), 2.40 (s, 3H), 4.20 (d, J=17.2 Hz, 1H), 4.50-4.56 (m, 2H), 4.83 (dd, J=11.4, 5.0 Hz, 2H), 4.99 (dd, J=11.4, 8.3 Hz, 1H), 5.90 (dd, J=8.3, 5.0 Hz, 1H), 6.49 (dt, J=7.6, 0.7 Hz, 1H), 6.68 (dt, J=7.8, 0.7 Hz, 1H), 6.86 (d, J=8.8 Hz, 2H), 7.31-7.35 (m, 3H), 7.38-7.41 (m, 2H), 7.76 (dd, J=8.1, 0.4 Hz, 1H). ¹³C NMR (125 MHz, CDCl₃) δ 22.2., 24.2, 47.0, 53.5, 64.6, 70.0, 107.8, 116.1, 116.3, 123.2, 125.1, 128.6, 129.1, 129.1, 131.4, 137.7, 139.0, 143.3, 156.2, 157.8, 162.2, 167.7. HRMS (APCI) m/z calculated for C₂₅H₂₆ClN₂O₃ [M+H]⁺437.16265, found 437.16173.

### Example 18. Synthesis and Characterization of 2-(1-(4-isopropoxyphenyl)-2-((6-methylpyridin-2-yl)oxy)ethyl-6-(trifluoromethyl)isoindolin-1-one (EU1180-511)

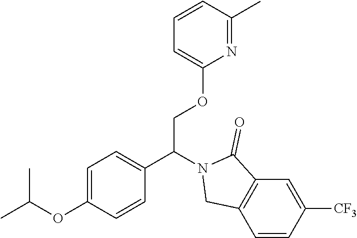 [SMILES: Cc1cccc(OCC(c2ccc(OC(C)C)cc2)N2Cc3ccc(C(F)(F)F)cc3C2=O)n1]

Synthesized according to General Procedure C using 1-(4-isopropoxyphenyl)-2-((6-methylpyridin-2-yl)oxy)ethan-1-amine (1 eq., 0.482 mmol, 138 mg), methyl 2-(bromomethyl)-5-(trifluoromethyl)benzoate (1.2 eq., 0.578 mmol, 172 mg), triethylamine (2.1 eq., 1.01 mmol, 141 μL), and MeOH (6 mL). Yield: 96 mg (0.204 mmol, 42%). ¹H NMR (500 MHz, CDCl₃) δ 1.32 (dd, J=6.1, 3.4 Hz, 6H), 2.38 (s, 3H), 4.29 (d, J=17.5 Hz, 1H), 4.52 (sep, J=6.1 Hz, 1H), 4.63 (d, J=17.5 Hz, 1H), 4.89 (dd, J=11.5, 4.9 Hz, 1H), 5.01 (dd, J=11.5, 8.4 Hz, 1H), 5.94 (dd, J=8.4, 4.9 Hz, 1H), 6.49 (d, J=8.2 Hz, 1H), 6.66 (d, J=7.2 Hz, 1H), 6.87 (d, J=8.8 Hz, 2H), 7.34 (d, J=8.5 Hz, 2H), 7.39 (dd, J=8.2, 7.3 Hz, 2H), 7.49 (d, J=8.0 Hz, 1H), 7.73 (dd, J=8.0, 1.0 Hz, 1H), 8.11 (s, 1H). ¹³C NMR (125 MHz, CDCl₃) δ 22.1, 24.1, 47.4, 53.6, 64.5, 70.0, 107.8, 116.2, 116.3, 121.2 (q, J_{CF}=3.8 Hz), 123.5, 124.0 (q, J_{CF}=279 Hz), 128.1 (q, J_{CF}=3.5 Hz), 128.9, 129.1, 130.9 (q, J_{CF}=33 Hz), 133.7, 139.0, 145.1, 156.1, 157.8, 162.6, 167.4. HRMS (APCI) m/z calculated for C₂₆H₂₆F₃N₂O₃ [M+H]⁺471.18900, found 471.18815.

### Example 19. Synthesis and Characterization of 6-fluoro-2-(1-(4-isopropoxyphenyl)-2-((6-methylpyridin-2-yl)oxy)ethyl)isoindolin-1-one (EU1180-512)

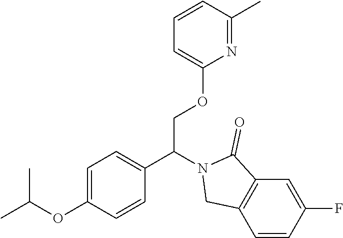 [SMILES: Cc1cccc(OCC(c2ccc(OC(C)C)cc2)N2Cc3ccc(F)cc3C2=O)n1]

Synthesized according to General Procedure C using 1-(4-isopropoxyphenyl)-2-((6-methylpyridin-2-yl)oxy)ethan-1-amine (1 eq., 0.454 mmol, 130 mg), methyl 2-(bromomethyl)-5-fluorobenzoate (1.2 eq., 0.545 mmol, 135 mg), triethylamine (2.1 eq., 0.953 mmol, 133 μL), and MeOH (6 mL). Yield: 41 mg (0.098 mmol, 22%). ¹H NMR (500 MHz, CDCl₃) δ 1.32 (dd, J=6.1, 3.1 Hz, 6H), 2.39 (s, 3H), 4.19 (d, J=16.7 Hz, 1H), 4.50-4.55 (m, 2H), 4.84 (dd, J=11.4, 5.0 Hz, 1H), 4.99 (dd, J=11.4, 8.3 Hz, 1H), 5.91 (dd, J=8.3, 5.0 Hz, 1H), 6.50 (dt, J=8.3, 0.7 Hz, 1H), 6.67 (dt, J=7.2, 0.7 Hz, 1H), 6.87 (d, J=8.8 Hz, 2H), 7.18 (ddd, J=9.0, 8.3, 2.5 Hz, 1H), 7.30-7.35 (m, 3H), 7.40 (dd, J=8.2, 7.2 Hz, 1H), 7.50 (dd, J=7.6, 2.4 Hz, 1H). ¹³C NMR (125 MHz, CDCl₃) δ 22.1, 24.2, 46.9, 53.6, 64.6, 70.0, 107.9, 110.6 (d, J_{CF}=23.2 Hz), 116.1, 116.2, 118.8 (d, J_{CF}=23.4 Hz), 124.2 (d, J_{CF}=8.2 Hz), 129.1, 129.2, 135.0 (d, J_{CF}=10.8 Hz), 137.1 (d, J_{CF}=1.9 Hz), 139.0, 156.2, 157.8, 162.7, 162.9 (d, J_{CF}=260 Hz), 167.7 (d, J_{CF}=3.7 Hz). HRMS (APCI) m/z calculated for C₂₅H₂₆FN₂O₃ [M+H]⁺421.19220, found 421.19118.

### Example 20. Synthesis and Characterization of 5-fluoro-2-(1-(4-isopropoxyphenyl)-2-((6-methylpyridin-2-yl)oxy)ethyl)isoindolin-1-one (EU1180-513)

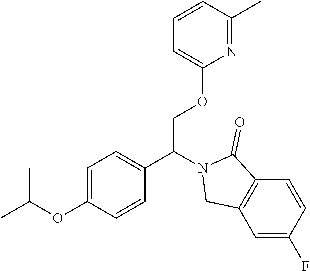 [SMILES: Cc1cccc(OCC(c2ccc(OC(C)C)cc2)N2Cc3cc(F)ccc3C2=O)n1]

Synthesized according to General Procedure C using 1-(4-isopropoxyphenyl)-2-((6-methylpyridin-2-yl)oxy)ethan-1-amine (1 eq., 0.454 mmol, 130 mg), methyl 2-(bromomethyl)-4-fluorobenzoate (1.2 eq., 0.545 mmol, 135 mg), triethylamine (2.1 eq., 0.953 mmol, 133 μL), and MeOH (6 mL). Yield: 41 mg (0.098 mmol, 22%). ¹H NMR (500 MHz, CDCl₃) δ 1.32 (dd, J=6.1, 3.1 Hz, 6H), 2.40 (s, 3H), 4.20 (d, J=17.1 Hz, 1H), 4.50-4.56 (m, 2H), 4.83 (dd, J=11.4, 5.1 Hz, 1H), 4.99 (dd, J=11.4, 8.2 Hz, 1H), 5.90 (dd, J=8.2, 5.0 Hz, 1H), 6.50 (d, J=8.3 Hz, 1H), 6.67 (d, J=7.3 Hz, 1H), 6.86 (d, J=8.8 Hz, 2H), 7.05 (dd, J=8.2, 2.0 Hz, 1H), 7.11 (td, J=8.8, 2.0 Hz, 1H), 7.33 (d, J=8.5 Hz, 2H), 7.40 (dd, J=8.2, 7.3 Hz, 1H), 7.81 (dd, J=8.4, 5.0 Hz, 1H). ¹³C NMR (125 MHz, CDCl₃) δ 22.2, 24.2, 47.1, 53.5, 64.7, 70.0, 107.9, 110.2 (d, J_{CF}=24 Hz), 115.8 (d, J=24 Hz), 116.1, 116.3, 125.8 (d, J_{CF}=9.7 Hz), 128.9, 129.1, 129.2, 139.0, 144.1 (d, J_{CF}=9.8 Hz), 156.2, 157.7, 162.7, 165.1 (d, J_{CF}=255 Hz), 167.8. HRMS (APCI) m/z calculated for C₂₅H₂₆FN₂O₃ [M+H]⁺421.19220, found 421.19127.

### Example 21. Synthesis and Characterization of 6-bromo-2-(1-(4-isopropoxyphenyl)-2-((6-methylpyridin-2-yl)oxy)ethyl)isoindolin-1-one EU1180-514

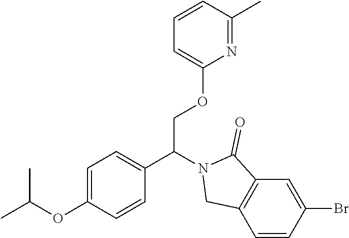 [SMILES: Cc1cccc(OCC(c2ccc(OC(C)C)cc2)N2Cc3ccc(Br)cc3C2=O)n1]

Synthesized according to General Procedure C using 1-(4-isopropoxyphenyl)-2-((6-methylpyridin-2-yl)oxy)ethan-1-amine (1 eq., 0.559 mmol, 160 mg), methyl 2-(bromomethyl)-5-bromobenzoate (1.2 eq., 0.670 mmol, 207 mg), triethylamine (2.1 eq., 1.17 mmol, 164 μL), and MeOH (8 mL). Yield: 223 mg (0.463 mmol, 83%). ¹H NMR (500 MHz, CDCl₃) δ 1.31 (dd, J=6.1, 3.1 Hz, 6H), 2.38 (s, 3H), 4.17 (d, J=17.2 Hz, 1H), 4.48-4.55 (m, 2H), 4.83 (dd, J=11.5, 5.0 Hz, 1H), 4.98 (dd, J=11.5, 8.4 Hz, 1H), 5.90 (dd, J=8.4, 5.0 Hz, 1H), 6.48 (d, J=8.3 Hz, 1H), 6.66 (d, J=7.2 Hz, 1H), 6.86 (d, J=8.8 Hz, 2H), 7.22 (dd, J=7.2, 0.4 Hz, 1H), 7.32 (d, J=8.5 Hz, 2H), 7.38 (dd, J=8.2, 7.3 Hz, 1H), 7.56 (dd, J=8.0, 1.9 Hz, 1H), 7.95 (d, J=1.9 Hz, 1H). ¹³C NMR (125 MHz, CDCl₃) δ 22.1, 24.1, 47.0, 53.5, 64.5, 69.9, 107.8, 115.9, 116.1, 116.2, 121.9, 124.3, 127.0, 129.0, 134.2, 134.9, 138.9, 140.3, 156.1, 157.7, 162.6, 167.2. HRMS (APCI) m/z calculated for C₂₅H₂₆BrN₂O₃ [M+H]⁺481.11213, found 481.11142.

### Example 22. Synthesis and Characterization of 5-bromo-2-(1-(4-isopropoxyphenyl)-2-((6-methylpyridin-2-yl)oxy)ethyl)isoindolin-1-one (EU1180-515)

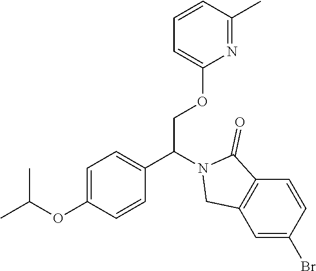 [SMILES: Cc1cccc(OCC(c2ccc(OC(C)C)cc2)N2Cc3cc(Br)ccc3C2=O)n1]

Synthesized according to General Procedure C using 1-(4-isopropoxyphenyl)-2-((6-methylpyridin-2-yl)oxy)ethan-1-amine (1 eq., 0.559 mmol, 160 mg), methyl 2-(bromomethyl)-4-bromobenzoate (1.2 eq., 0.670 mmol, 207 mg), triethylamine (2.1 eq., 1.17 mmol, 164 μL), and MeOH (8 mL). Yield: 223 mg (0.463 mmol, 83%). ¹H NMR (500 MHz, CDCl₃) δ 1.30 (dd, J=6.1, 3.2 Hz, 6H), 2.38 (s, 3H), 4.19 (d, J=17.2 Hz, 1H), 4.47-4.54 (m, 2H), 4.83 (dd, J=11.5, 5.0 Hz, 1H), 4.98 (dd, J=11.4, 8.4 Hz, 1H), 5.89 (dd, J=8.3, 5.0 Hz, 1H), 6.48 (d, J=8.3 Hz, 1H), 6.65 (d, J=7.2 Hz, 1H), 6.86 (d, J=8.8 Hz, 2H), 7.32 (d, J=8.6 Hz, 1H), 7.37 (dd, J=8.2, 7.3 Hz, 1H), 7.48 (d, J=1.0 Hz, 1H), 7.52 (dd, J=8.1, 1.7 Hz, 1H), 7.67 (d, J=8.1 Hz, 1H). ¹³C NMR (125 MHz, CDCl₃) δ 22.1, 24.1, 46.8, 53.4, 64.5, 69.9, 107.7, 116.0, 116.2, 125.2, 125.9, 126.1, 129.0, 131.3, 131.7, 138.9, 143.5, 156.0, 157.7, 162.5, 167.7. HRMS (APCI) m/z calculated for C₂₅H₂₆BrN₂O₃ [M+H]⁺481.11213, found 481.11165.

### Example 23. Synthesis and Characterization of 2-(2-(4-ethoxyphenoxy)-1-(4-isopropoxyphenyl)ethyl)isoindolin-1-one (EU1180-499)

EU1180-499 was synthesized according to the steps shown in Scheme 3 below.

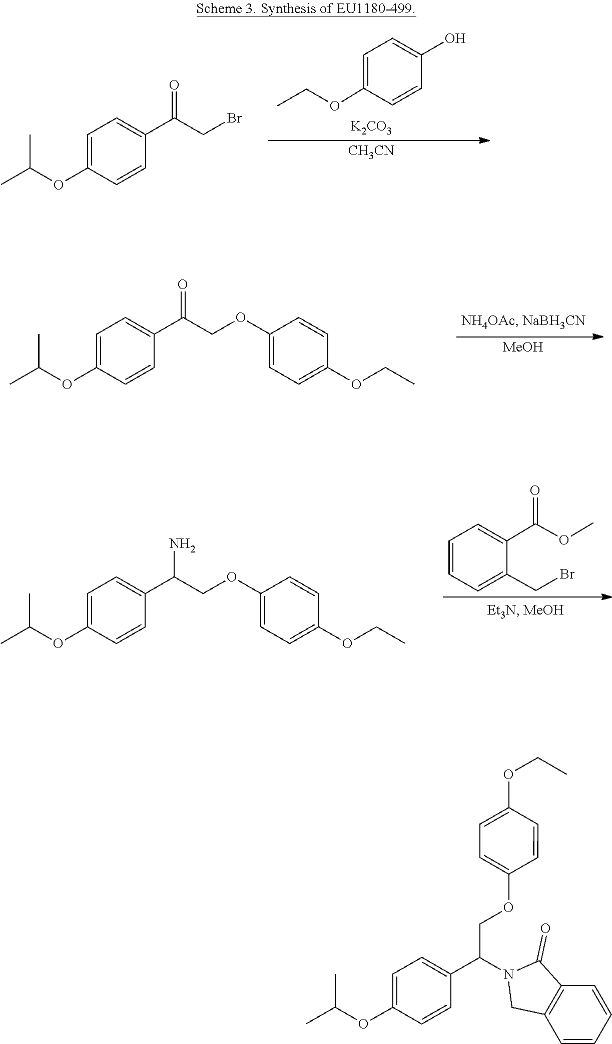

### A. Synthesis of 2-(4-ethoxyphenoxy)-1-(4-isopropoxyphenyl)ethan-1-one

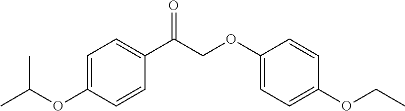 [SMILES: CCOc1ccc(OCC(=O)c2ccc(OC(C)C)cc2)cc1]

A solution of 2-bromo-4′-isopropoxyacetophenone (1 eq., 4.67 mmol, 1.2 g) in acetonitrile (25 mL) was supplemented with crushed anhydrous potassium carbonate (1.3 eq., 6.07 mmol, 839 mg) and 4-ethoxyphenol (1.2 eq., 5.60 mmol, 695 mg), and the reaction mixture was stirred at room temperature overnight. The next day, the mixture was filtered through cotton/sand, and the acetonitrile was evaporated under reduced pressure. The product was purified by flash column chromatography (silica gel, 0-40% ethyl acetate in hexanes) to afford the product as a colorless oil. Yield: 1.42 g (4.51 mmol, 97%).

### B. Synthesis of 2-(4-ethoxyphenoxy)-1-(4-isopropoxyphenyl)ethan-1-amine

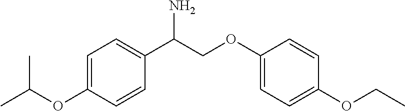 [SMILES: CCOc1ccc(OCC(N)c2ccc(OC(C)C)cc2)cc1]

Synthesized according to General Procedure B using 2-(4-ethoxyphenoxy)-1-(4-isopropoxyphenyl)ethan-1-one (1 eq., 10.05 mmol, 3.16 g), NaCNBH₃ (6 eq., 60.3 mmol, 3.79 g), NH₄HCO₂ (10 eq., 100.5 mmol, 6.34 g), and EtOH (50 mL). Yield: 2.43 g (7.70 mmol, 77%).

### C. Synthesis of 2-(1-(4-isopropoxyphenyl)-2-(4-methoxyphenoxy)ethyl)isoindolin-1-one

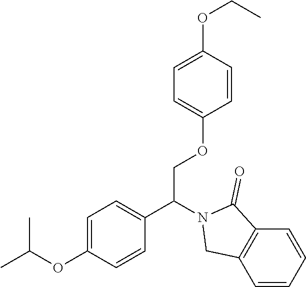 [SMILES: CCOc1ccc(OCC(c2ccc(OC(C)C)cc2)N2Cc3ccccc3C2=O)cc1]

Synthesized according to General Procedure C using 2-(4-ethoxyphenoxy)-1-(4-isopropoxyphenyl)ethan-1-amine (1 eq., 0.381 mmol, 120 mg), methyl 2-(bromomethyl)benzoate (1.2 eq., 0.457 mmol, 105 mg), DIPEA (2.1 eq., 0.798 mmol, 111 μL), and MeOH (5 mL). Yield: 75 mg (0.174 mmol, 46%). ¹H NMR (500 MHz, CHCl₃) δ 1.31 (dt, J=6.0, 3.0 Hz, 6H), 1.38 (t, J=7.0 Hz, 3H), 3.97 (q, J=7.0 Hz, 2H), 4.25 (d, J=17.0 Hz, 1H), 4.47 (dd, J=10.0, 5.1 Hz, 1H), 4.50-4.58 (m, 3H), 5.86 (t, J=5.8 Hz, 1H), 6.80-6.88 (m, 6H), 7.34 (d, J=8.6 Hz, 2H), 7.38 (dt, J=7.5 Hz, 0.9 Hz, 1H), 7.45 (td, J=7.5, 1.1 Hz, 1H), 7.50 (td, J=7.4, 1.2 Hz, 1H), 7.89 (d, J=7.2 Hz, 1H). ¹³C NMR (125 MHz, CHCl₃) δ 25.1, 22.2, 48.0, 53.5, 64.2, 68.9, 70.0, 115.6, 115.9, 116.1, 122.9, 124.0, 128.0, 129.3, 131.4, 131.9, 132.8, 141.9, 152.6, 153.7, 168.8. HRMS (APCI) m/z calculated for C₂₇H₃₀NO₄ [M+H]⁺432.21693, found 432.21740.

### Example 24. Synthesis and Characterization of 2-(2-(4-ethoxyphenoxy)-1-(4-isopropoxyphenyl)ethyl)-6-(trifluoromethyl)isoindolin-1-one EU1180-587)

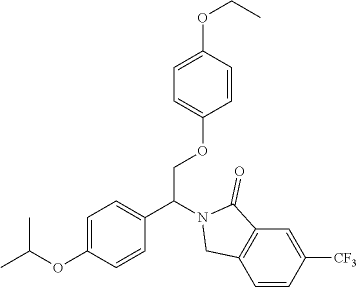 [SMILES: CCOc1ccc(OCC(c2ccc(OC(C)C)cc2)N2Cc3ccc(C(F)(F)F)cc3C2=O)cc1]

Synthesized according to General Procedure D using 2-(4-ethoxyphenoxy)-1-(4-isopropoxyphenyl)ethan-1-amine (1 eq., 0.476 mmol, 150 mg), methyl 2-(bromomethyl)-5-(trifluoromethyl)benzoate (1.2 eq., 0.571 mmol, 170 mg), DIPEA (2.5 eq., 1.19 mmol, 207 μL), and EtOH (5 mL). Yield: 111 mg (0.222 mmol, 47%). ¹H NMR (600 MHz, CDCl₃) δ 1.32 (t, J=5.1 Hz, 6H), 1.37 (t, J=7.0 Hz, 3H), 3.96 (q, J=7.0 Hz, 2H), 4.32 (d, J=17.6 Hz, 1H), 4.48 (dd, J=10.0, 4.9 Hz, 1H), 4.53 (sep, J=6.1 Hz, 1H), 4.59 (dd, J=10.1, 6.7 Hz, 1H), 4.63 (d, J=17.6 Hz, 1H), 5.86 (t, J=5.7 Hz, 1H), 6.81 (d, J=9.1 Hz, 2H), 6.87 (t, J=8.7 Hz, 4H), 7.35 (d, J=8.7 Hz, 2H), 7.51 (d, J=7.9 Hz, 1H), 7.76 (d, J=8.0 Hz, 1H), 8.16 (s, 1H). ¹³C NMR (150 MHz, CDCl₃) δ 15.0, 22.1, 48.0, 53.7, 64.1, 68.8, 70.0, 115.6, 115.9, 116.1, 121.2 (q, J_{CF}=3.9 Hz), 123.1, 124.0 (q, J_{CF}=272 Hz), 128.2 (q, J_{CF}=3.2 Hz), 128.7, 129.2, 130.9 (q, J_{CF}=33 Hz), 133.5, 145.2, 152.4, 153.8, 157.8, 167.4. HRMS (APCI) m/z calculated for C₂₈H₂₉F₃NO₄ [M+H]⁺ 500.20432, found 500.20509.

### Example 25. Synthesis and Characterization of 6-chloro-2-(2-(4-ethoxyphenoxy)-1-(4-isopropoxyphenyl)ethyl)isoindolin-1-one (EU1180-588)

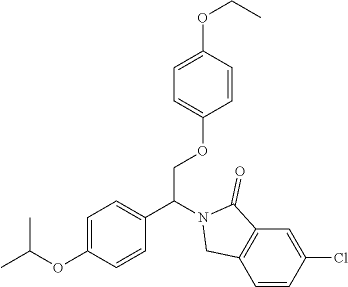 [SMILES: CCOc1ccc(OCC(c2ccc(OC(C)C)cc2)N2Cc3ccc(Cl)cc3C2=O)cc1]

Synthesized according to General Procedure D using 2-(4-ethoxyphenoxy)-1-(4-isopropoxyphenyl)ethan-1-amine (1 eq., 0.476 mmol, 150 mg), methyl 2-(bromomethyl)-5-chlorobenzoate (1.2 eq., 0.571 mmol, 151 mg), DIPEA (2.5 eq., 1.19 mmol, 207 μL), and EtOH (5 mL). Yield: 123 mg (0.264 mmol, 55%). ¹H NMR (600 MHz, CDCl₃) δ 1.32 (dd, J=5.9, 4.0 Hz, 6H), 1.37 (t, J=7.0 Hz, 3H), 3.96 (q, J=7.0 Hz, 2H), 4.22 (d, J=17.2 Hz, 1H), 4.45 (dd, J=10.0, 5.0 Hz, 1H), 4.49-4.58 (m, 3H), 5.82 (t, J=5.8 Hz, 1H), 6.81 (d, J=9.1 Hz, 2H), 6.83-6.88 (m, 4H), 7.30 (d, J=8.1 Hz, 1H), 7.33 (d, J=8.6 Hz, 2H), 7.45 (d, J=8.6, Hz, 1H), 7.84 (d, J=1.9 Hz, 1H). ¹³C NMR (150 MHz, CDCl₃) δ 15.0, 22.1, 47.6, 53.7, 64.1, 68.7, 69.9, 115.5, 115.9, 116.1, 124.0, 124.1, 128.8, 129.9, 131.5, 134.3, 134.5, 139.9, 152.4, 153.7, 157.8, 167.4. HRMS (APCI) m/z calculated for C₂₇H₂₉ClNO₄ [M+H]⁺ 466.17796, found 466.17886.

### Example 26. Synthesis and Characterization of 5-chloro-2-(2-(4-ethoxyphenoxy)-1-(4-isopropoxyphenyl)ethyl)isoindolin-1-one (EU1180-589)

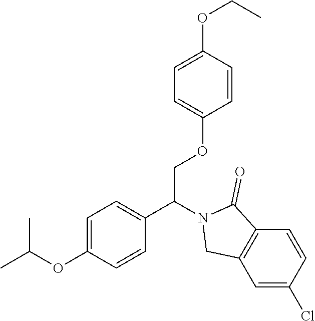 [SMILES: CCOc1ccc(OCC(c2ccc(OC(C)C)cc2)N2Cc3cc(Cl)ccc3C2=O)cc1]

Synthesized according to General Procedure D using 2-(4-ethoxyphenoxy)-1-(4-isopropoxyphenyl)ethan-1-amine (1 eq., 0.476 mmol, 150 mg), methyl 2-(bromomethyl)-4-chlorobenzoate (1.2 eq., 0.571 mmol, 151 mg), DIPEA (2.5 eq., 1.19 mmol, 207 μL), EtOH (5 mL). Yield: 116 mg (0.249 mmol, 52%). ¹H NMR (600 MHz, CDCl₃) δ 1.32 (dd, J=5.9, 4.1 Hz, 6H), 1.37 (t, J=7.0 Hz, 3H), 3.96 (q, J=7.0 Hz, 2H), 4.22 (d, J=17.3 Hz, 1H), 4.45 (dd, J=10.0, 5.0 Hz, 1H), 4.50-4.58 (m, 3H), 5.82 (t, J=5.8 Hz, 1H), 6.81 (d, J=9.1 Hz, 2H), 6.83-6.88 (m, 4H), 7.31-7.35 (m, 3H), 7.41 (d, J=8.5 Hz, 1H), 7.79 (d, J=8.1, Hz, 1H), 7.84 (d, J=1.9 Hz, 1H). ¹³C NMR (150 MHz, CDCl₃) δ 15.0, 22.1, 47.5, 53.6, 64.0, 68.8, 69.9, 115.5, 115.8, 116.1, 123.3, 125.0, 128.6, 128.9, 129.2, 131.2, 137.7, 143.4, 152.4, 153.7, 157.7, 167.7. HRMS (APCI) m/z calculated for C₂₇H₂₉ClNO₄ [M+H]⁺ 466.17796, found 466.17897.

### Example 27. Synthesis and Characterization of 6-fluoro-2-(2-(4-ethoxyphenoxy)-1-(4-isopropoxyphenyl)ethyl)isoindolin-1-one (EU1180-590)

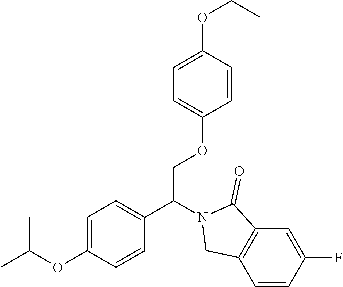 [SMILES: CCOc1ccc(OCC(c2ccc(OC(C)C)cc2)N2Cc3ccc(F)cc3C2=O)cc1]

Synthesized according to General Procedure D using 2-(4-ethoxyphenoxy)-1-(4-isopropoxyphenyl)ethan-1-amine (1 eq., 0.476 mmol, 150 mg), methyl 2-(bromomethyl)-5-fluorobenzoate (1.2 eq., 0.571 mmol, 141 mg), DIPEA (2.5 eq., 1.19 mmol, 207 μL), and EtOH (5 mL). Yield: 101 mg (0.225 mmol, 47%). ¹H NMR (600 MHz, CDCl₃) δ 1.32 (dd, J=6.0, 3.8 Hz, 6H), 1.37 (t, J=7.0 Hz, 3H), 3.96 (q, J=7.0 Hz, 2H), 4.22 (d, J=16.9 Hz, 1H), 4.46 (dd, J=10.0, 5.0 Hz, 1H), 4.49-4.58 (m, 3H), 5.83 (t, J=5.9 Hz, 1H), 6.81 (d, J=9.1 Hz, 2H), 6.84-6.88 (m, 4H), 7.20 (td, J=8.7, 2.4 Hz, 1H), 7.31-7.35 (m, 3H), 7.53 (dd, J=7.6, 2.4 Hz, 1H). ¹³C NMR (150 MHz, CDCl₃) δ 15.0, 22.1, 47.5, 53.7, 64.1, 68.8, 69.9, 110.5 (d, J_{CF}=23 Hz), 115.5, 115.9, 116.1, 118.9 (d, J_{CF}=24 Hz), 124.3 (d, J_{CF}=8.4 Hz), 128.9, 129.2, 134.8 (d, J_{CF}=8.4 Hz), 137.1, 152.4, 153.7, 157.8, 162.8 (d, J_{CF}=247 Hz), 167.7 (d, J_{CF}=3.7 Hz). HRMS (APCI) m/z calculated for C₂₇H₂₉FNO₄ [M+H]⁺ 450.20751, found 450.20826.

### Example 28. Synthesis and Characterization of 5-fluoro-2-(2-(4-ethoxyphenoxy)-1-(4-isopropoxyphenyl)ethyl)isoindolin-1-one (EU1180-591)

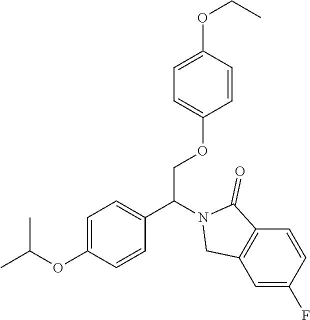 [SMILES: CCOc1ccc(OCC(c2ccc(OC(C)C)cc2)N2Cc3cc(F)ccc3C2=O)cc1]

Synthesized according to General Procedure D using 2-(4-ethoxyphenoxy)-1-(4-isopropoxyphenyl)ethan-1-amine (1 eq., 0.476 mmol, 150 mg), methyl 2-(bromomethyl)-4-fluorobenzoate (1.2 eq., 0.571 mmol, 141 mg), DIPEA (2.5 eq., 1.19 mmol, 207 μL), EtOH (5 mL). Yield: 90 mg (0.200 mmol, 42%). ¹H NMR (600 MHz, CDCl₃) δ 1.32 (dd, J=6.0, 4.0 Hz, 6H), 1.37 (t, J=7.0 Hz, 3H), 3.96 (q, J=7.0 Hz, 2H), 4.23 (d, J=17.2 Hz, 1H), 4.46 (dd, J=10.0, 5.0 Hz, 1H), 4.50-4.58 (m, 3H), 5.82 (t, J=5.7 Hz, 1H), 6.81 (d, J=9.1 Hz, 2H), 6.84-6.88 (m, 4H), 7.05 (dd, J=8.0, 1.6 Hz, 1H), 7.14 (td, J=8.9, 2.0 Hz, 1H), 7.33 (d, J=8.7 Hz, 2H), 7.85 (dd, J=8.4, 5.0 Hz, 1H). ¹³C NMR (150 MHz, CDCl₃) δ 15.0, 22.1, 47.7, 53.6, 64.1, 68.8, 70.0, 110.2 (d, J_{CF}=24 Hz), 115.6, 115.8 (d, J_{CF}=23 Hz), 115.9, 116.1, 125.8 (d, J_{CF}=9.8 Hz), 128.8, 129.0, 129.2, 144.2 (d, J_{CF}=10.1 Hz), 152.4, 153.7, 157.7, 165.1 (d, J_{CF}=251 Hz), 167.7. HRMS (APCI) m/z calculated for C₂₇H₂₉FNO₄ [M+H]⁺450.20751, found 450.20820.

### Example 29. Synthesis and Characterization of 6-bromo-2-(2-(4-ethoxyphenoxy)-1-(4-isopropoxyphenyl)ethyl)isoindolin-1-one (EU1180-592)

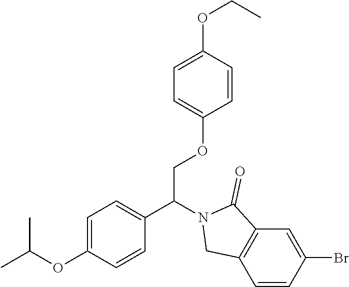 [SMILES: CCOc1ccc(OCC(c2ccc(OC(C)C)cc2)N2Cc3ccc(Br)cc3C2=O)cc1]

Synthesized according to General Procedure D using 2-(4-ethoxyphenoxy)-1-(4-isopropoxyphenyl)ethan-1-amine (1 eq., 0.476 mmol, 150 mg), methyl 2-(bromomethyl)-5-bromobenzoate (1.2 eq., 0.571 mmol, 176 mg), DIPEA (2.5 eq., 1.19 mmol, 207 μL), and EtOH (5 mL). Yield: 127 mg (0.249 mmol, 52%). ¹H NMR (600 MHz, CDCl₃) δ 1.31 (dd, J=6.0, 3.9 Hz, 6H), 1.37 (t, J=7.0 Hz, 3H), 3.96 (q, J=7.0 Hz, 2H), 4.20 (d, J=17.2 Hz, 1H), 4.43-4.57 (m, 4H), 5.82 (t, J=5.8 Hz, 1H), 6.80 (d, J=9.1 Hz, 2H), 6.83-6.88 (m, 4H), 7.24 (d, J=8.0 Hz, 1H), 7.33 (d, J=8.7 Hz, 2H), 7.59 (dd, J=8.0, 1.6 Hz, 1H), 7.99 (s, 1H). ¹³C NMR (150 MHz, CDCl₃) δ 15.0, 22.1, 47.7, 53.6, 64.0, 68.7, 69.9, 115.5, 115.8, 116.1, 122.0, 124.4, 127.0, 128.8, 129.2, 134.3, 134.8, 140.4, 152.4, 153.7, 157.7, 167.2. HRMS (APCI) m/z calculated for C₂₇H₂₉BrNO₄ [M+H]⁺510.12745, found 510.12870.

### Example 30. Synthesis and Characterization of 5-bromo-2-(2-(4-ethoxyphenoxy)-1-(4-isopropoxyphenyl)ethyl)isoindolin-1-one

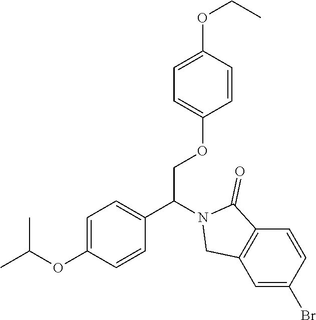 [SMILES: CCOc1ccc(OCC(c2ccc(OC(C)C)cc2)N2Cc3cc(Br)ccc3C2=O)cc1]

Synthesized according to General Procedure D using 2-(4-ethoxyphenoxy)-1-(4-isopropoxyphenyl)ethan-1-amine (1 eq., 0.476 mmol, 150 mg), methyl 2-(bromomethyl)-5-bromobenzoate (1.2 eq., 0.571 mmol, 176 mg), DIPEA (2.5 eq., 1.19 mmol, 207 μL), and EtOH (5 mL). Yield: 123 mg (0.241 mmol, 51%). ¹H NMR (600 MHz, CDCl₃) δ 1.32 (dd, J=6.0, 4.2 Hz, 6H), 1.38 (t, J=7.0 Hz, 3H), 3.96 (q, J=7.0 Hz, 2H), 4.22 (d, J=17.3 Hz, 1H), 4.45 (dd, J=10.1, 5.0 Hz, 1H), 4.50-4.58 (m, 4H), 5.81 (t, J=5.7 Hz, 1H), 6.81 (d, J=9.1 Hz, 2H), 6.83-6.87 (m, 4H), 7.32 (d, J=8.7 Hz, 2H), 7.52 (s, 1H), 7.58 (dd, J=8.1, 1.3 Hz, 1H), 7.73 (d, J=8.1 Hz, 1H). ¹³C NMR (150 MHz, CDCl₃) δ 14.9, 22.0, 47.4, 53.5, 64.0, 68.7, 69.9, 115.5, 115.8, 116.0, 125.2, 126.0, 126.1, 128.8, 129.1, 131.4, 131.6, 143.5, 152.3, 153.6, 157.7, 167.7. HRMS (APCI) m/z calculated for C₂₇H₂₉BrNO₄ [M+H]⁺ 510.12745, found 510.12856.

### Example 31. Synthesis and Characterization of 2-(1-(4-isopropoxyphenyl)-2-(4-methoxyphenoxy)ethyl)-6-(trifluoromethyl)isoindolin-1-one (EU1180-558)

EU1180-558 was synthesized according to the steps shown in Scheme 4 below.

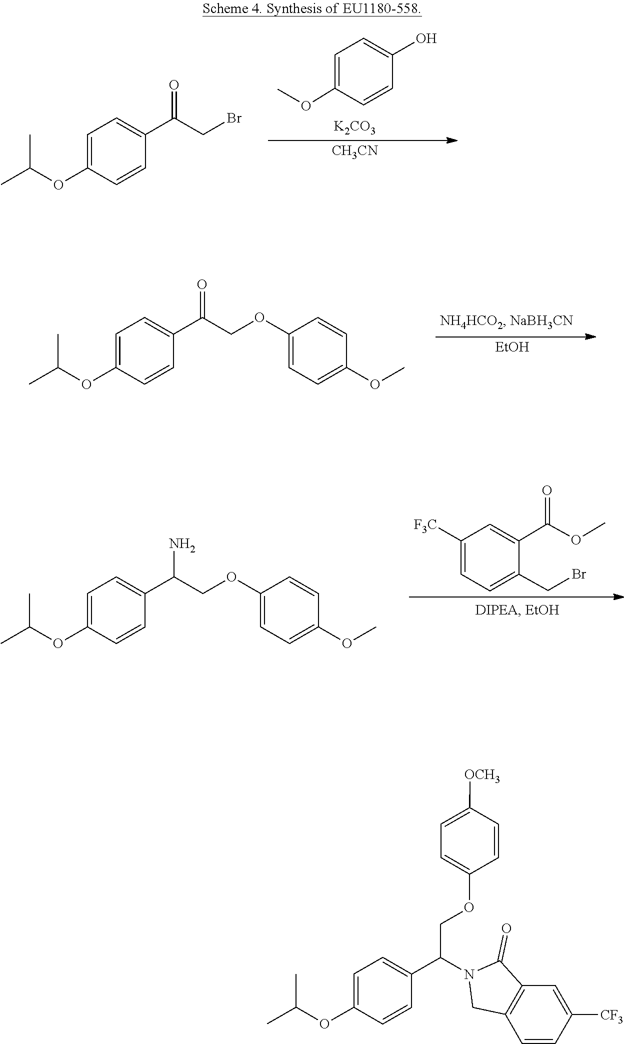

### A. Synthesis of 1-(4-isopropoxyphenyl)-2-(4-methoxyphenoxy)ethan-1-one

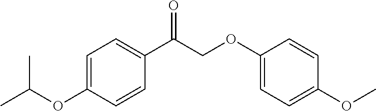 [SMILES: COc1ccc(OCC(=O)c2ccc(OC(C)C)cc2)cc1]

A solution of 2-bromo-4′-isopropoxyacetophenone (1 eq., 4.67 mmol, 1.2 g) in acetonitrile (25 mL) was supplemented with crushed anhydrous potassium carbonate (1.3 eq., 6.07 mmol, 839 mg) and 4-methoxyphenol (1.2 eq., 5.60 mmol, 695 mg). The reaction mixture was stirred at room temperature overnight. The next day, the mixture was filtered through cotton/sand, and the acetonitrile was evaporated under reduced pressure. The product was purified by flash column chromatography (silica gel, 0-40% ethyl acetate in hexanes) to afford the product as a colorless oil. Yield: 1.39 g (4.63 mmol, 99%).

### B. Synthesis of 1-(4-isopropoxyphenyl)-2-(4-methoxyphenoxy)ethan-1-amine

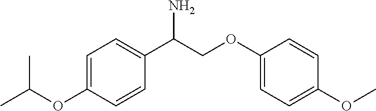 [SMILES: COc1ccc(OCC(N)c2ccc(OC(C)C)cc2)cc1]

Synthesized according to General Procedure B using 1-(4-isopropoxyphenyl)-2-(4-methoxyphenoxy)ethan-1-one (1 eq., 11.1 mmol, 3.34 g), NaCNBH₃ (6 eq., 66.7 mmol, 4.19 g), NH₄HCO₂ (10 eq., 111.2 mmol, 7.01 g), and EtOH (60 mL). Yield: 2.54 mg (8.43 mmol, 76%).

### C. Synthesis of 2-(1-(4-isopropoxyphenyl)-2-(4-methoxyphenoxy)ethyl)-6-(trifluoromethyl)isoindolin-1-one

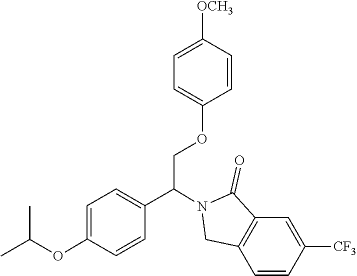 [SMILES: COc1ccc(OCC(c2ccc(OC(C)C)cc2)N2Cc3ccc(C(F)(F)F)cc3C2=O)cc1]

Synthesized according to General Procedure D using 1-(4-isopropoxyphenyl)-2-(4-methoxyphenoxy)ethan-1-amine (1 eq., 0.465 mmol, 140 mg), methyl 2-(bromomethyl)-5-(trifluoromethyl)benzoate (1.2 eq., 0.557 mmol, 166 mg), DIPEA (2.5 eq., 1.16 mmol, 203 μL), and EtOH (6 mL). Yield: 133 mg (0.274 mmol, 59%). ¹H NMR (500 MHz, CDCl₃) δ 1.32 (dd, J=6.1, 3.5 Hz, 6H), 3.74 (s, 3H), 4.32 (d, J=17.7 Hz, 1H), 4.48 (dd, J=10.1, 5.0 Hz, 1H), 4.53 (sep, J=6.1 Hz, 1H), 4.57-4.65 (m, 2H), 5.86 (t, J=5.9 Hz, 1H), 6.81 (d, J=9.2 Hz, 2H), 6.85-6.89 (m, 4H), 7.35 (d, J=8.6 Hz, 2H), 7.51 (d, J=8.0 Hz, 1H), 7.75 (dd, J=8.0, 1.1 Hz, 1H), 8.15 (s, 1H). ¹³C NMR (125 MHz, CDCl₃) δ 22.1, 47.9, 53.8, 55.7, 68.8, 70.0, 114.8, 115.9, 116.2, 121.1 (q, J_{CF}=3.8 Hz), 123.5, 124.0 (q, J_{CF}=277 Hz), 128.1 (q, J_{CF}=3.6 Hz), 128.7, 129.2, 130.9 (q, J_{CF}=33 Hz), 133.5, 145.2, 152.4, 154.5, 157.9, 167.3. HRMS (APCI) m/z calculated for C₂₇H₂₇F₃NO₄ [M+H]⁺ 486.18867 found 486.18810.

### Example 32. Synthesis and Characterization of 6-chloro-2-(1-(4-isopropoxyphenyl)-2-(4-methoxyphenoxy)ethyl)isoindolin-1-one (EU1180-564)

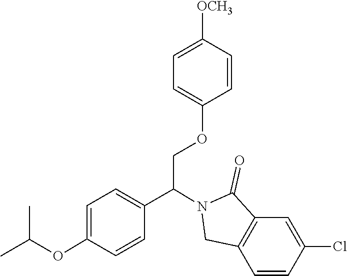 [SMILES: COc1ccc(OCC(c2ccc(OC(C)C)cc2)N2Cc3ccc(Cl)cc3C2=O)cc1]

Synthesized according to General Procedure D using 1-(4-isopropoxyphenyl)-2-(4-methoxyphenoxy)ethan-1-amine (1 eq., 0.465 mmol, 140 mg), methyl 2-(bromomethyl)-5-chlorobenzoate (1.2 eq., 0.557 mmol, 147 mg), DIPEA (2.5 eq., 1.16 mmol, 203 μL), and EtOH (6 mL). Yield: 114 mg (0.252 mmol, 54%). ¹H NMR (500 MHz, CDCl₃) δ 1.32 (dd, J=6.1, 3.3 Hz, 6H), 3.74 (s, 3H), 4.22 (d, J=17.3 Hz, 1H), 4.45 (dd, J=10.0, 5.1 Hz, 4H), 4.49-4.58 (m, 3H), 5.82 (t, J=5.9 Hz, 1H), 6.81 (d, J=9.3 Hz, 2H), 6.86 (d, J=9.1 Hz, 4H), 7.29 (d, J=8.1 Hz, 1H), 7.33 (d, J=8.6 Hz, 2H), 7.45 (dd, J=8.1, 2.0 Hz, 1H), 7.83 (d, J=1.7, 1H). ¹³C NMR (125 MHz, CDCl₃) δ 22.1, 47.6, 53.7, 55.8, 68.8, 70.0, 114.8, 115.9, 116.1, 124.0, 124.1, 128.8, 129.2, 131.5, 134.3, 134.5, 139.9, 152.5, 154.4, 157.8, 167.4. HRMS (APCI) m/z calculated for C₂₆H₂₇ClNO₄ [M+H]⁺ 452.16231 found 452.16175.

### Example 33. Synthesis and Characterization of 5-chloro-2-(1-(4-isopropoxyphenyl)-2-(4-methoxyphenoxy)ethyl)isoindolin-1-one (EU1180-559)

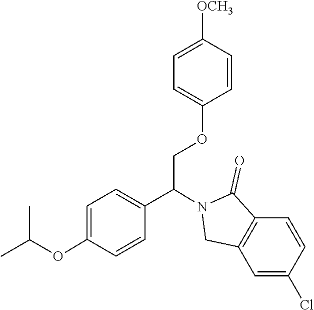 [SMILES: COc1ccc(OCC(c2ccc(OC(C)C)cc2)N2Cc3cc(Cl)ccc3C2=O)cc1]

Synthesized according to General Procedure D using 1-(4-isopropoxyphenyl)-2-(4-methoxyphenoxy)ethan-1-amine (1 eq., 0.465 mmol, 140 mg), methyl 2-(bromomethyl)-4-chlorobenzoate (1.2 eq., 0.557 mmol, 147 mg), DIPEA (2.5 eq., 1.16 mmol, 203 μL), and EtOH (6 mL). Yield: 114 mg (0.253 mmol, 55%). ¹H NMR (500 MHz, CDCl₃) δ 1.32 (dd, J=6.1, 3.4 Hz, 6H), 3.74 (s, 3H), 4.22 (d, J=17.3 Hz, 1H), 4.46 (dd, J=10.0, 5.0 Hz, 1H), 4.50-4.58 (m, 3H), 5.82 (t, J=5.8 Hz, 1H), 6.81 (d, J=9.3 Hz, 2H), 6.86 (d, J=8.9 Hz, 4H), 7.31-7.35 (m, 3H), 7.41 (dd, J=8.1, 1.8 Hz, 1H), 7.79 (d, J=8.2 Hz, 1H). ¹³C NMR (125 MHz, CDCl₃) δ 22.1, 47.5, 53.6, 55.8, 68.8, 70.0, 114.8, 115.9, 116.1, 123.3, 125.0, 128.6, 128.9, 129.2, 131.2, 137.7, 143.4, 152.5, 154.4, 157.8, 167.7. HRMS (APCI) m/z calculated for C₂₆H₂₇ClNO₄ [M+H]⁺ 452.16231 found 452.16170.

### Example 34. Synthesis and Characterization of 6-fluoro-2-(1-(4-isopropoxyphenyl)-2-(4-methoxyphenoxy)ethyl)isoindolin-1-one (EU1180-560)

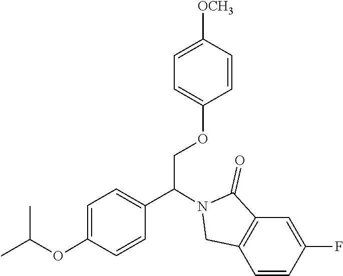 [SMILES: COc1ccc(OCC(c2ccc(OC(C)C)cc2)N2Cc3ccc(F)cc3C2=O)cc1]

Synthesized according to General Procedure D using 1-(4-isopropoxyphenyl)-2-(4-methoxyphenoxy)ethan-1-amine (1 eq., 0.465 mmol, 140 mg), methyl 2-(bromomethyl)-5-fluorobenzoate (1.2 eq., 0.557 mmol, 138 mg), DIPEA (2.5 eq., 1.16 mmol, 203 μL), and EtOH (6 mL). Yield: 130 mg (0.299 mmol, 64%). ¹H NMR (500 MHz, CDCl₃) δ 1.32 (dd, J=6.1, 3.2 Hz, 6H), 3.74 (s, 3H), 4.22 (d, J=16.9 Hz, 1H), 4.46 (dd, J=10.0, 5.1 Hz, 1H), 4.49-4.59 (m, 3H), 5.83 (t, J=5.9 Hz, 1H), 6.81 (d, J=9.1 Hz, 2H), 6.87 (d, J=8.0 Hz, 4H), 7.19 (td, J=8.6, 2.1 Hz, 1H), 7.31-7.34 (m, 3H), 7.53 (dd, J=7.6, 2.2 Hz, 1H). ¹³C NMR (125 MHz, CDCl₃) δ 22.1, 47.5, 53.7, 55.8, 68.8, 70.0, 110.5 (d, J_{CF}=23.5 Hz), 114.8, 115.9, 116.1, 118.9 (d, J_{CF}=23.6 Hz), 124.3 (d, J_{CF}=8.2 Hz), 128.9, 129.2, 134.8 (d, J_{CF}=8.5 Hz), 137.1 (d, J_{CF}=2.5 Hz), 152.5, 154.4, 157.8, 162.9 (d, J_{CF}=246 Hz), 167.7 (d, J_{CF}=3.5 Hz). HRMS (APCI) m/z calculated for C₂₆H₂₇FON₄ [M+H]⁺ 436.19186 found 436.19128.

### Example 35. Synthesis and Characterization of 5-fluoro-2-(1-(4-isopropoxyphenyl)-2-(4-methoxyphenoxy)ethyl)isoindolin-1-one (EU1180-561)

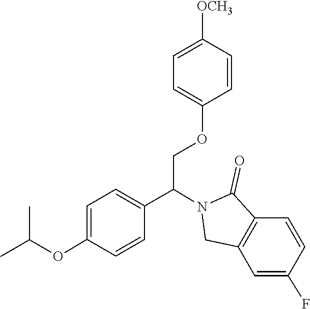 [SMILES: COc1ccc(OCC(c2ccc(OC(C)C)cc2)N2Cc3cc(F)ccc3C2=O)cc1]

Synthesized according to General Procedure D using 1-(4-isopropoxyphenyl)-2-(4-methoxyphenoxy)ethan-1-amine (1 eq., 0.531 mmol, 160 mg), methyl 2-(bromomethyl)-4-fluorobenzoate (1.2 eq., 0.637 mmol, 157 mg), DIPEA (2.5 eq., 1.16 mmol, 231 μL), and EtOH (6 mL). Yield: 182 mg (0.418 mmol, 79%). ¹H NMR (500 MHz, CDCl₃) δ 1.32 (dd, J=6.3, 3.2 Hz, 6H), 3.74 (s, 3H), 4.23 (d, J=17.3 Hz, 1H), 4.48-4.58 (m, 3H), 5.82 (t, J=5.8 Hz, 1H), 6.81 (d, J=9.2 Hz, 2H), 6.85-6.88 (m, 4H), 7.05 (dd, J=8.2, 2.0 Hz, 1H), 7.13 (td, J=8.8, 2.0 Hz, 1H), 7.33 (d, J=8.5 Hz, 2H), 7.84 (dd, J=8.4, 5.0 Hz, 1H). ¹³C NMR (125 MHz, CDCl₃) δ 22.1, 47.6, 53.6, 55.7, 68.8, 69.9, 110.2 (d, J_{CF}=24.1 Hz), 114.8, 115.7, 115.9, 116.1, 125.8 (d, J_{CF}=9.7 Hz), 128.8, 128.9, 129.2, 144.1 (d, J_{CF}=9.9 Hz), 152.5, 154.4, 157.7, 165.1 (d, J_{CF}=250 Hz), 167.7. HRMS (APCI) m/z calculated for C₂₆H₂₇FON₄ [M+H]⁺ 436.19186 found 436.19133.

### Example 36. Synthesis and Characterization of 6-bromo-2-(1-(4-isopropoxyphenyl)-2-(4-methoxyphenoxy)ethyl)isoindolin-1-one (EU1180-562)

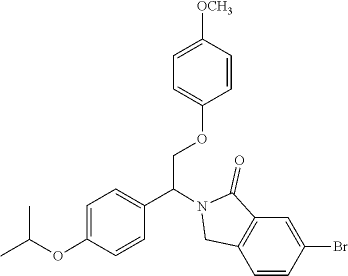 [SMILES: COc1ccc(OCC(c2ccc(OC(C)C)cc2)N2Cc3ccc(Br)cc3C2=O)cc1]

Synthesized according to General Procedure D using 1-(4-isopropoxyphenyl)-2-(4-methoxyphenoxy)ethan-1-amine (1 eq., 0.465 mmol, 140 mg), methyl 2-(bromomethyl)-5-bromobenzoate (1.2 eq., 0.557 mmol, 172 mg), DIPEA (2.5 eq., 1.16 mmol, 203 μL), and EtOH (6 mL). Yield: 117 mg (0.236 mmol, 51%). ¹H NMR (500 MHz, CDCl₃) δ 1.32 (dd, J=6.1, 3.3 Hz, 6H), 3.74 (s, 3H), 4.20 (d, J=17.3 Hz, 1H), 4.43-4.59 (m, 4H), 5.82 (t, J=5.8 Hz, 1H), 6.81 (d, J=9.2 Hz, 2H), 6.86 (d, J=9.0 Hz, 4H), 7.24 (d, J=8.1 Hz, 2H), 7.32 (d, J=8.8 Hz, 1H), 7.60 (d, J=8.0 Hz, 1H), 7.99 (s, 1H). ¹³C NMR (125 MHz, CDCl₃) δ 22.1, 47.7, 53.7, 55.8, 68.8, 70.0, 114.8, 115.9, 116.1, 122.1, 124.4, 127.0, 128.8, 129.2, 134.3, 134.8, 140.4, 152.5, 154.4, 157.8, 167.3. HRMS (APCI) m/z calculated for C₂₆H₂₇BrNO₄ [M+H]⁺ 496.11180 found 496.11137.

### Example 37. Synthesis and Characterization of 5-bromo-2-(1-(4-isopropoxyphenyl)-2-(4-methoxyphenoxy)ethyl)isoindolin-1-one (EU1180-563)

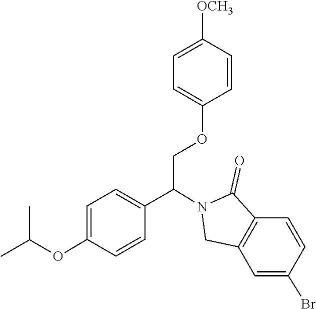 [SMILES: COc1ccc(OCC(c2ccc(OC(C)C)cc2)N2Cc3cc(Br)ccc3C2=O)cc1]

Synthesized according to General Procedure D using 1-(4-isopropoxyphenyl)-2-(4-methoxyphenoxy)ethan-1-amine (1 eq., 0.498 mmol, 150 mg), methyl 2-(bromomethyl)-4-bromobenzoate (1.2 eq., 0.597 mmol, 184 mg), DIPEA (2.5 eq., 1.25 mmol, 217 μL), and EtOH (6 mL). Yield: 61 mg (0.123 mmol, 25%). ¹H NMR (500 MHz, CDCl₃) δ 1.32 (dd, J=6.1, 3.5 Hz, 6H), 3.75 (s, 3H), 4.22 (d, J=17.3 Hz, 1H), 4.45 (dd, J=10.0, 5.0 Hz, 4H), 4.50-4.58 (m, 3H), 5.82 (t, J=5.8 Hz, 1H), 6.81 (d, J=9.3 Hz, 2H), 6.84-6.88 (m, 4H), 7.32 (d, J=8.4 Hz, 2H), 7.52 (s, 1H), 7.58 (dd, J=8.1, 1.6 Hz, 1H), 7.73 (d, J=8.4, 1H). ¹³C NMR (125 MHz, CDCl₃) δ 22.1, 47.5, 53.6, 55.8, 68.8, 70.0, 114.9, 115.9, 116.1, 125.3, 126.1, 126.3, 128.9, 129.2, 131.5, 131.7, 143.6, 152.5, 154.4, 157.8, 167.8. HRMS (APCI) m/z calculated for C₂₆H₂₇BrNO₄ [M+H]⁺ 496.11180 found 496.11120.

### Example 38. Synthesis and Characterization of 2-(1-(4-isopropoxyphenyl)-2-(4-(trifluoromethoxy)phenoxy)ethyl)isoindolin-1-one (EU1180-538)

EU1180-538 was synthesized according to the steps shown in Scheme 5 below.

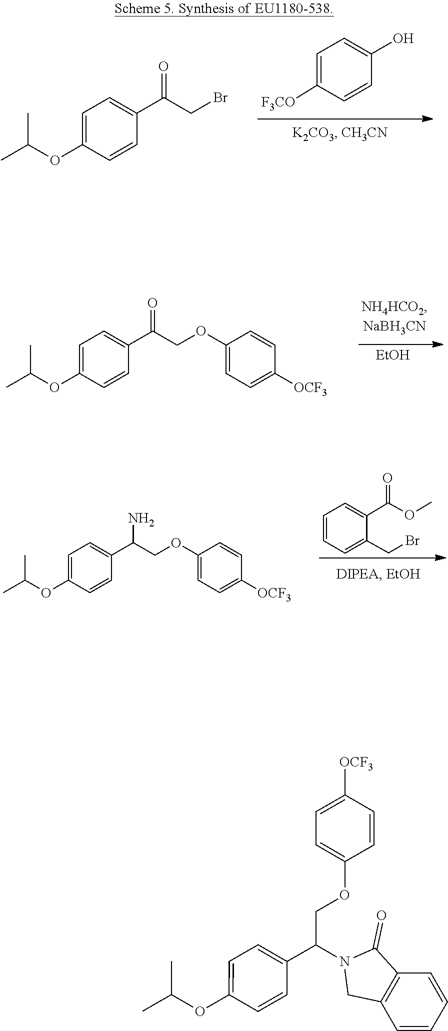

### A. Synthesis of 1-(4-isopropoxyphenyl)-2-(4-(trifluoromethoxy)phenoxy)ethan-1-one

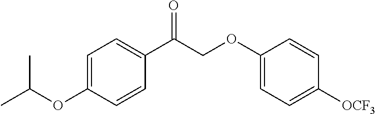 [SMILES: CC(C)Oc1ccc(C(=O)COc2ccc(OC(F)(F)F)cc2)cc1]

A solution of 2-bromo-4′-isopropoxyacetophenone (1 eq., 9.72 mmol, 2.50 g) in acetonitrile (50 mL) was supplemented with 4-(trifluoromethoxy)phenol (1.2 eq., 11.67 mmol, 2.08 g) and anhydrous K₂CO₃ (1.5 eq., 14.58 mmol, 2.02 g). After stirring the reaction mixture at room temperature overnight, the mixture was filtered through cotton/sand, and the acetonitrile was removed under reduced pressure. The crude mixture was then loaded onto a silica gel column with dichloromethane, and the product was purified by flash column chromatography (0-20% ethyl acetate in hexanes) to afford a white solid. Yield: 3.14 g (8.85 mmol, 91%).

### B. Synthesis of 1-(4-isopropoxyphenyl)-2-(4-(trifluoromethoxy)phenoxy)ethan-1-amine

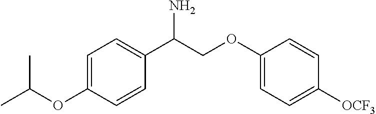 [SMILES: CC(C)Oc1ccc(C(N)COc2ccc(OC(F)(F)F)cc2)cc1]

Synthesized according to General Procedure B using 1-(4-isopropoxyphenyl)-2-(4-(trifluoromethoxy)phenoxy)ethan-1-one (1 eq., 6.72 mmol, 2.38 g), NaCNBH₃ (6 eq., 40.3 mmol, 2.53 g), NH₄HCO₂ (10 eq., 67.2 mmol, 4.24 g), EtOH (50 mL). Yield: 2.01 g (5.66 mmol, 84%).

### C. Synthesis of 2-(1-(4-isopropoxyphenyl)-2-(4-(trifluoromethoxy)phenoxy)ethyl)isoindolin-1-one

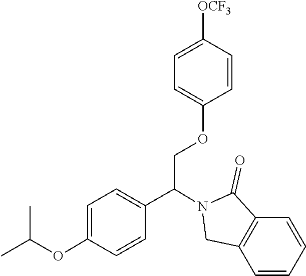 [SMILES: CC(C)Oc1ccc(C(COc2ccc(OC(F)(F)F)cc2)N2Cc3ccccc3C2=O)cc1]

Synthesized according to General Procedure D using 1-(4-isopropoxyphenyl)-2-(4-(trifluoromethoxy)phenoxy)ethan-1-amine (1 eq., 0.366 mmol, 130 mg), methyl 2-(bromomethyl)benzoate (1.2 eq., 0.439 mmol, 101 mg), DIPEA (2.5 eq., 0.915 mmol, 159 μL), and EtOH (5 mL). Yield: 65 mg (0.139 mmol, 38%). ¹H NMR (600 MHz, CDCl₃) δ 1.33 (dd, J=5.9, 4.2 Hz, 6H), 4.24 (d, J=16.9 Hz, 1H), 4.47-4.56 (m, 3H), 4.62 (dd, J=9.9, 6.6 Hz, 1H), 5.86 (t, J=5.9 Hz, 1H), 6.87 (d, J=8.7 Hz, 2H), 6.93 (d, J=9.1 Hz, 2H), 7.13 (d, J=8.9, 2H), 7.34 (d, J=8.7 Hz, 2H), 7.38 (d, J=7.5 Hz, 1H), 7.46 (t, J=7.4 Hz, 1H), 7.51 (t, J=8.2 Hz, 1H), 7.89 (d, J=7.5 Hz, 1H). ¹³C NMR (125 MHz, CDCl₃) δ 22.0, 47.8, 53.4, 68.3, 69.9, 115.5, 116.0, 120.5 (q, J_{CF}=260 Hz), 122.5, 122.8, 123.9, 128.0, 128.6, 129.1, 131.4, 132.6, 141.6, 143.2, 156.8, 157.7, 168.7. HRMS (APCI) m/z calculated for C₂₆H₂₅F₃NO₄ [M+H]⁺ 572.17302, found 572.17259.

### Example 39. Synthesis and Characterization of 2-(1-(4-isopropoxyphenyl)-2-(4-(trifluoromethoxy)phenoxy)ethyl)-6-(trifluoromethyl)isoindolin-1-one (EU1180-539)

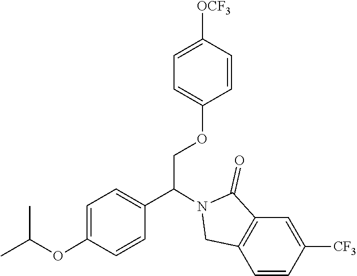 [SMILES: CC(C)Oc1ccc(C(COc2ccc(OC(F)(F)F)cc2)N2Cc3ccc(C(F)(F)F)cc3C2=O)cc1]

Synthesized according to General Procedure D using 1-(4-isopropoxyphenyl)-2-(4-(trifluoromethoxy)phenoxy)ethan-1-amine (1 eq., 0.366 mmol, 130 mg), methyl 2-(bromomethyl)-5-(trifluoromethyl)benzoate (1.2 eq., 0.439 mmol, 130 mg), DIPEA (2.5 eq., 0.915 mmol, 159 μL), and EtOH (5 mL). Yield: 107 mg (0.198 mmol, 54%). ¹H NMR (500 MHz, CDCl₃) δ 1.33 (dd, J=6.1, 3.5 Hz, 6H), 4.32 (d, J=17.4 Hz, 1H), 4.50-4.72 (m, 4H), 5.87 (t, J=5.9 Hz, 1H), 6.89 (d, J=8.8 Hz, 2H), 6.93 (d, J=9.2 Hz, 2H), 7.13 (d, J=9.1, 2H), 7.34 (d, J=8.6 Hz, 2H), 7.52 (t, J=8.1 Hz, 1H), 7.77 (d, J=7.9 Hz, 1H), 8.15 (s, 1H). ¹³C NMR (125 MHz, CDCl₃) δ 22.1, 48.0, 53.8, 68.4, 70.1, 114.8, 115.7, 116.3, 120.6 (q, J_{CF}=263 Hz), 121.3 (q, J_{CF}=3.8 Hz), 122.6, 123.9 (q, J_{CF}=277 Hz), 127.1 (q, J_{CF}=3.8 Hz), 128.3 (q, J_{CF}=3.9 Hz), 129.2, 131.1 (q, J_{CF}=33 Hz), 133.5, 143.4, 145.1, 156.8, 158.1, 167.4. HRMS (APCI) m/z calculated for C₂₇H₂₄F₆NO₄ [M+H]⁺ 540.16040, found 540.15990.

### Example 40. Synthesis and Characterization of 6-chloro-2-(1-(4-isopropoxyphenyl)-2-(4-(trifluoromethoxy)phenoxy)ethyl)isoindolin-1-one (EU1180-540)

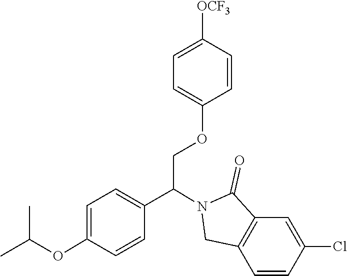 [SMILES: CC(C)Oc1ccc(C(COc2ccc(OC(F)(F)F)cc2)N2Cc3ccc(Cl)cc3C2=O)cc1]

Synthesized according to General Procedure D using 1-(4-isopropoxyphenyl)-2-(4-(trifluoromethoxy)phenoxy)ethan-1-amine (1 eq., 0.366 mmol, 130 mg), methyl 2-(bromomethyl)-5-chlorobenzoate (1.2 eq., 0.439 mmol, 116 mg), DIPEA (2.5 eq., 0.915 mmol, 159 μL), and EtOH (5 mL). Yield: 48 mg (0.095 mmol, 26%). ¹H NMR (500 MHz, CDCl₃) δ 1.33 (dd, J=6.1, 3.3 Hz, 6H), 4.22 (d, J=17.2 Hz, 1H), 4.45-4.67 (m, 4H), 5.84 (t, J=5.9 Hz, 1H), 6.88 (d, J=8.8 Hz, 2H), 6.92 (d, J=9.2 Hz, 2H), 7.13 (dd, J=9.2, 0.8 Hz, 2H), 7.30-7.35 (m, 3H), 7.47 (td, J=8.1, 2.0 Hz, 1H), 7.84 (d, J=2.0 Hz, 1H). ¹³C NMR (125 MHz, CDCl₃) δ 22.1, 47.7, 53.8, 68.4, 70.1, 114.8, 115.7, 116.2, 120.7 (q, J_{CF}=267 Hz), 122.6, 124.1, 127.1 (d, J_{CF}=3.6 Hz), 128.4, 129.2, 131.7, 134.4 (q, J_{CF}=4.5 Hz), 139.8, 143.3, 156.9, 158.0, 167.5. HRMS (APCI) m/z calculated for C₂₆H₂₄ClF₃NO₄ [M+H]⁺ 506.13405 found 506.13357.

### Example 41. Synthesis and Characterization of 5-chloro-2-(1-(4-isopropoxyphenyl)-2-(4-(trifluoromethoxy)phenoxy)ethyl)isoindolin-1-one (EU1180-541)

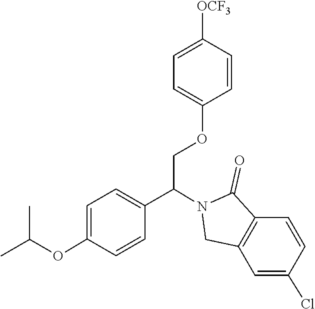 [SMILES: CC(C)Oc1ccc(C(COc2ccc(OC(F)(F)F)cc2)N2Cc3cc(Cl)ccc3C2=O)cc1]

Synthesized according to General Procedure D using 1-(4-isopropoxyphenyl)-2-(4-(trifluoromethoxy)phenoxy)ethan-1-amine (1 eq., 0.366 mmol, 130 mg), methyl 2-(bromomethyl)-4-chlorobenzoate (1.2 eq., 0.439 mmol, 116 mg), DIPEA (2.5 eq., 0.915 mmol, 159 μL), and EtOH (5 mL). Yield: 74 mg (0.146 mmol, 40%). ¹H NMR (500 MHz, CDCl₃) δ 1.33 (dd, J=6.1, 3.4 Hz, 6H), 4.22 (d, J=17.2 Hz, 1H), 4.45-4.70 (m, 4H), 5.83 (t, J=5.9 Hz, 1H), 6.87 (d, J=8.8 Hz, 2H), 6.92 (d, J=9.2 Hz, 2H), 7.13 (d, J=9.1 Hz, 2H), 7.32 (d, J=8.8 Hz, 2H), 7.36 (s, 1H), 7.43 (dd, J=8.1, 1.7 Hz, 1H), 7.80 (d, J=8.1 Hz, 1H). ¹³C NMR (125 MHz, CDCl₃) δ 22.1, 47.6, 53.7, 68.4, 70.1, 114.8, 115.7, 116.2, 120.6 (q, J_{CF}=256 Hz), 122.7, 123.3, 125.2, 127.1 (q, J_{CF}=3.7 Hz), 128.4, 128.8, 129.2 131.2, 137.9, 143.3, 156.8, 158.0, 167.8. HRMS (APCI) m/z calculated for C₂₆H₂₄ClF₃NO₄ [M+H]⁺ 506.13405 found 506.13385.

### Example 42. Synthesis and Characterization of 6-fluoro-2-(1-(4-isopropoxyphenyl)-2-(4-(trifluoromethoxy)phenoxy)ethyl)isoindolin-1-one (EU1180-542)

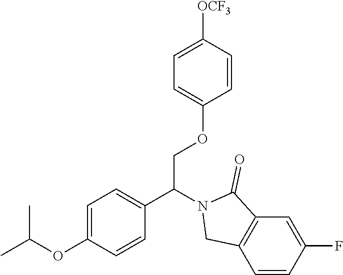 [SMILES: CC(C)Oc1ccc(C(COc2ccc(OC(F)(F)F)cc2)N2Cc3ccc(F)cc3C2=O)cc1]

Synthesized according to General Procedure D using 1-(4-isopropoxyphenyl)-2-(4-(trifluoromethoxy)phenoxy)ethan-1-amine (1 eq., 0.366 mmol, 130 mg), methyl 2-(bromomethyl)-5-fluorobenzoate (1.2 eq., 0.439 mmol, 109 mg), DIPEA (2.5 eq., 0.915 mmol, 159 μL), and EtOH (5 mL). Yield: 79 mg (0.161 mmol, 44%). ¹H NMR (500 MHz, CDCl₃) δ 1.33 (dd, J=6.1, 3.3 Hz, 6H), 4.22 (d, J=16.8 Hz, 1H), 4.46-4.64 (m, 4H), 5.85 (t, J=6.0 Hz, 1H), 6.88 (d, J=8.8 Hz, 2H), 6.93 (d, J=9.2 Hz, 2H), 7.13 (d, J=9.2, 0.8 Hz, 2H), 7.21 (td, J=8.7, 2.4 Hz, 3H), 7.34 (d, J=8.6 Hz, 1H), 7.54 (dd, J=7.5, 2.5 Hz, 1H). ¹³C NMR (125 MHz, CDCl₃) δ 22.1, 47.6, 53.8, 68.4, 70.0, 110.7 (d, J=23 Hz), 114.8, 115.7, 116.2, 119.1 (d, J_{CF}=24 Hz), 120.7 (q, J_{CF}=260 Hz), 122.6, 124.4 (d, J_{CF}=8.3 Hz), 127.1 (q, J_{CF}=3.6 Hz), 128.5, 129.2, 134.7 (d, J_{CF}=11 Hz), 137.0(5) (d, J_{CF}=1.8 Hz), 143.3, 156.9, 158.0, 162.9 (d, J_{CF}=260 Hz), 167.8. HRMS (APCI) m/z calculated for C₂₆H₂₄F₄NO₄ [M+H]⁺ 490.16360 found 490.16310.

### Example 43. Synthesis and Characterization of 5-fluoro-2-(1-(4-isopropoxyphenyl)-2-(4-(trifluoromethoxy)phenoxy)ethyl)isoindolin-1-one (EU1180-544)

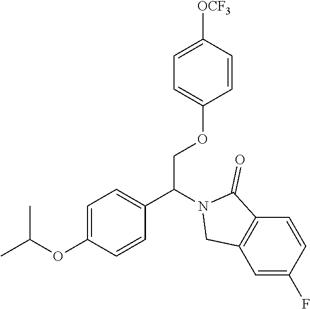 [SMILES: CC(C)Oc1ccc(C(COc2ccc(OC(F)(F)F)cc2)N2Cc3cc(F)ccc3C2=O)cc1]

Synthesized according to General Procedure D using 1-(4-isopropoxyphenyl)-2-(4-(trifluoromethoxy)phenoxy)ethan-1-amine (1 eq., 0.366 mmol, 130 mg), methyl 2-(bromomethyl)-4-fluorobenzoate (1.2 eq., 0.439 mmol, 109 mg), DIPEA (2.5 eq., 0.915 mmol, 159 μL), and EtOH (5 mL). Yield: 99 mg (0.202 mmol, 55%). ¹H NMR (500 MHz, CDCl₃) δ 1.33 (dd, J=6.1, 3.3 Hz, 6H), 4.22 (d, J=17.2 Hz, 1H), 4.46-4.70 (m, 4H), 5.83 (t, J=5.9 Hz, 1H), 6.88 (d, J=8.8 Hz, 2H), 6.93 (d, J=9.2 Hz, 2H), 7.06 (dd, J=8.1, 1.8 Hz, 1H), 7.11-7.15 (m, 2H), 7.33 (d, J=8.5 Hz, 2H), 7.85 (dd, J=8.4, 5.0 Hz, 1H). ¹³C NMR (125 MHz, CDCl₃) δ 22.1, 47.7, 53.6, 68.5, 70.0, 110.3 (d, J_{CF}=24 Hz), 114.8, 115.7, 116.0 (d, J_{CF}=24 Hz), 116.2, 120.6 (q, J_{CF}=256 Hz), 122.6, 125.9 (d, J_{CF}=9.7 Hz), 127.1 (q, J_{CF}=3.7 Hz), 128.5 (t, J_{CF}=20.0 Hz), 129.2, 143.3, 144.1 (d, J_{CF}=9.9 Hz), 156.9, 157.9, 165.2 (d, J_{CF}=250 Hz), 167.8. HRMS (APCI) m/z calculated for C₂₆H₂₄F₄NO₄ [M+H]⁺ 490.16360 found 490.16304.

### Example 44. Synthesis and Characterization of 6-bromo-2-(1-(4-isopropoxyphenyl)-2-(4-(trifluoromethoxy)phenoxy)ethyl)isoindolin-1-one (EU1180-553)

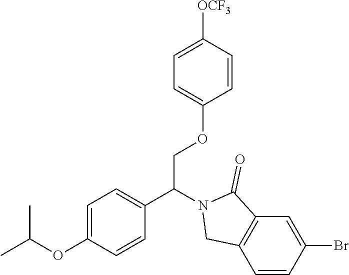 [SMILES: CC(C)Oc1ccc(C(COc2ccc(OC(F)(F)F)cc2)N2Cc3ccc(Br)cc3C2=O)cc1]

Synthesized according to General Procedure D using 1-(4-isopropoxyphenyl)-2-(4-(trifluoromethoxy)phenoxy)ethan-1-amine (1 eq., 0.394 mmol, 140 mg), methyl 2-(bromomethyl)-5-bromobenzoate (1.2 eq., 0.473 mmol, 146 mg), DIPEA (2.5 eq., 0.985 mmol, 172 μL), and EtOH (5 mL). Yield: 91 mg (0.165 mmol, 42%). ¹H NMR (500 MHz, CDCl₃) δ 1.33 (dd, J=6.1, 3.3 Hz, 6H), 4.20 (d, J=17.2 Hz, 1H), 4.43-4.70 (m, 4H), 5.84 (t, J=6.0 Hz, 1H), 6.88 (d, J=8.8 Hz, 2H), 6.92 (d, J=9.2 Hz, 2H), 7.13 (dd, J=9.1, 0.8 Hz, 2H), 7.25 (d, J=8.1 Hz, 1H), 7.32 (d, J=8.5 Hz, 2H), 7.61 (dd, J=7.6, 1.9 Hz, 1H), 7.85 (dd, J=8.4, 5.0 Hz, 1H), 8.00 (d, J=1.7 Hz, 1H). ¹³C NMR (125 MHz, CDCl₃) δ 22.1, 47.7, 53.7, 68.4, 70.0, 114.8, 115.7, 116.2, 120.6 (q, J_{CF}=259 Hz), 122.2, 122.6, 124.5, 127.1, 128.4, 129.2, 134.5, 134.7, 140.3, 143.3, 156.8, 158.0, 167.4. HRMS (APCI) m/z calculated for C₂₆H₂₄BrF₃NO₄ [M+H]⁺ 550.08353 found 550.08324.

### Example 45. Synthesis and Characterization of 5-bromo-2-(1-(4-isopropoxyphenyl)-2-(4-(trifluoromethoxy)phenoxy)ethyl)isoindolin-1-one (EU1180-554)

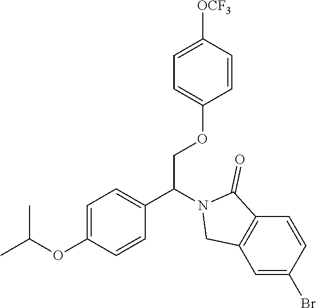 [SMILES: CC(C)Oc1ccc(C(COc2ccc(OC(F)(F)F)cc2)N2Cc3cc(Br)ccc3C2=O)cc1]

Synthesized according to General Procedure D using 1-(4-isopropoxyphenyl)-2-(4-(trifluoromethoxy)phenoxy)ethan-1-amine (1 eq., 0.394 mmol, 140 mg), methyl 2-(bromomethyl)-4-bromobenzoate (1.2 eq., 0.473 mmol, 146 mg), DIPEA (2.5 eq., 0.985 mmol, 172 μL), and EtOH (5 mL). Yield: 145 mg (0.263 mmol, 67%). ¹H NMR (500 MHz, CDCl₃) δ 1.33 (dd, J=6.1, 3.4 Hz, 6H), 4.22 (d, J=17.2 Hz, 1H), 4.45-4.70 (m, 4H), 5.83 (t, J=5.9 Hz, 1H), 6.87 (d, J=8.8 Hz, 2H), 6.92 (d, J=9.2 Hz, 2H), 7.13 (dd, J=9.2, 0.8 Hz, 2H), 7.32 (d, J=8.5 Hz, 2H), 7.51 (s, 1H), 7.58 (dd, J=8.1, 1.6 Hz, 1H), 7.72 (d, J=8.4 Hz, 1H). ¹³C NMR (125 MHz, CDCl₃) δ 22.1, 47.5, 53.6, 68.4, 70.0, 114.8, 115.6, 116.2, 120.6 (q, J_{CF}=256 Hz), 122.6, 123.7, 125.3, 126.2, 126.3, 127.1 (q, J_{CF}=3.7 Hz), 128.4, 129.2, 131.6, 143.3, 143.5, 156.8, 157.9, 167.9. HRMS (APCI) m/z calculated for C₂₆H₂₄BrF₃NO₄ [M+H]⁺ 550.08353 found 550.08321.

### Example 46. Synthesis and Characterization of 2-(1-(4-isopropoxyphenyl)-2-(4-(trifluoromethyl)phenoxy)ethyl)isoindolin-1-one (EU1180-571)

EU1180-571 was synthesized according to the steps shown in Scheme 6 below.

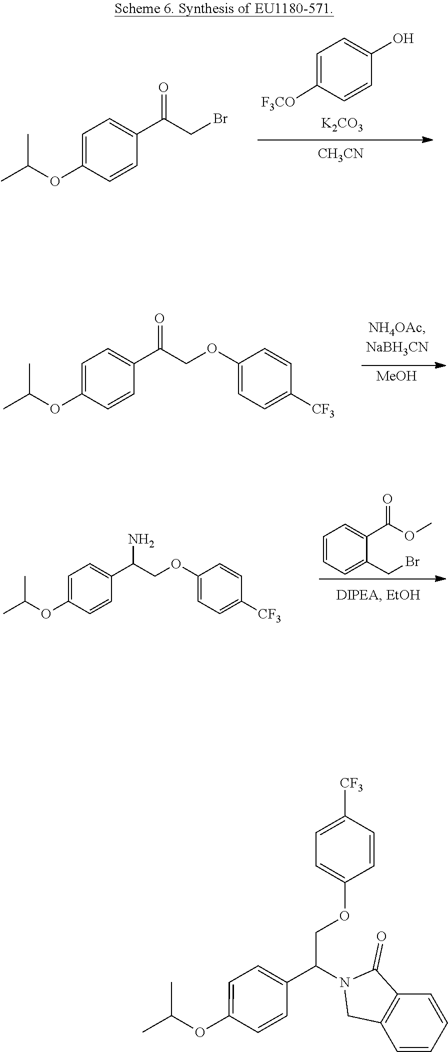

### A. Synthesis of 1-(4-isopropoxyphenyl)-2-(4-(trifluoromethyl)phenoxy)ethan-1-one

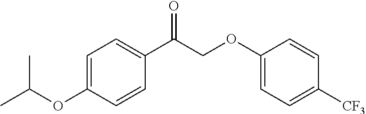 [SMILES: CC(C)Oc1ccc(C(=O)COc2ccc(C(F)(F)F)cc2)cc1]

A solution of 2-bromo-4′-isopropoxyacetophenone (1 eq., 9.72 mmol, 2.50 g) in acetonitrile (50 mL) was supplemented with 4-(trifluoromethyl)phenol (1.2 eq., 11.67 mmol, 1.89 g) and anhydrous K₂CO₃ (1.5 eq., 14.58 mmol, 2.02 g). After stirring the reaction mixture at room temperature overnight, the mixture was filtered through cotton/sand, and the acetonitrile was removed under reduced pressure. The crude mixture was then loaded onto a silica gel column with dichloromethane, and the product was purified by flash column chromatography (0-20% ethyl acetate in hexanes) to afford a white solid. Yield: 3.12 g (9.22 mmol, 95%).

### B. Synthesis of 1-(4-isopropoxyphenyl)-2-(4-(trifluoromethyl)phenoxy)ethan-1-amine

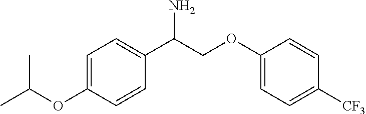 [SMILES: CC(C)Oc1ccc(C(N)COc2ccc(C(F)(F)F)cc2)cc1]

Synthesized according to General Procedure B using 1-(4-isopropoxyphenyl)-2-(4-(trifluoromethyl)phenoxy)ethan-1-one (1 eq., 8.57 mmol, 2.90 g), NaCNBH₃ (6 eq., 51.4 mmol, 3.23 g), NH₄HCO₂ (10 eq., 85.7 mmol, 5.41 g), and EtOH (40 mL). Yield: 2.72 g (8.02 mmol, 94%).

### C. Synthesis of 2-(1-(4-isopropoxyphenyl)-2-(4-(trifluoromethyl)phenoxy)ethyl)isoindolin-1-one

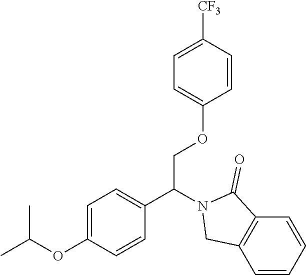 [SMILES: CC(C)Oc1ccc(C(COc2ccc(C(F)(F)F)cc2)N2Cc3ccccc3C2=O)cc1]

Synthesized according to General Procedure D using 1-(4-isopropoxyphenyl)-2-(4-(trifluoromethyl)phenoxy)ethan-1-amine (1 eq., 0.413 mmol, 140 mg), methyl 2-(bromomethyl)benzoate (1.2 eq., 0.495 mmol, 113 mg), DIPEA (2.5 eq., 1.03 mmol, 180 μL), and EtOH (4 mL). Yield: 57 mg (0.125 mmol, 30%). ¹H NMR (600 MHz, CDCl₃) δ 1.31 (dd, J=6.0, 3.4 Hz, 6H), 4.24 (d, J=16.9 Hz, 1H), 4.49 (d, J=16.9 Hz, 1H), 4.50-4.59 (m, 2H), 4.68 (dd, J=9.9, 6.7 Hz, 1H), 5.88 (t, J=6.0 Hz, 1H), 6.87 (d, J=8.7 Hz, 2H), 7.00 (d, J=8.7 Hz, 2H), 7.34 (d, J=8.7, 2H), 7.38 (d, J=7.6 Hz, 1H), 7.46 (t, J=7.4 Hz, 1H), 7.49-7.55 (m, 3H), 7.89 (dd, J=7.6, 0.6 Hz, 1H). ¹³C NMR (150 MHz, CDCl₃) δ 22.1, 48.0, 53.5, 68.1, 70.1, 114.8, 116.2, 122.9, 123.6 (q, J_{CF}=33 Hz), 124.0, 124.5 (q, J_{CF}=271 Hz), 127.1 (q, J_{CF}=3.6 Hz), 128.2, 128.6, 129.3, 131.6, 132.7, 141.7, 157.9, 160.9, 168.9. HRMS (APCI) m/z calculated for C₂₆H₂₅F₃NO₃ [M+H]⁺ 456.17810, found 456.17706.

### Example 47. Synthesis and Characterization of 2-(1-(4-isopropoxyphenyl)-2-(4-(trifluoromethyl)phenoxy)ethyl)-6-(trifluoromethyl)isoindolin-1-one (EU1180-572)

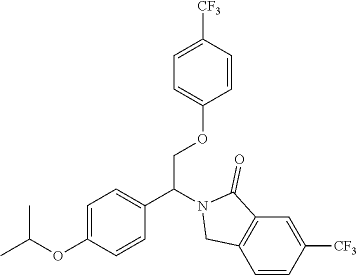 [SMILES: CC(C)Oc1ccc(C(COc2ccc(C(F)(F)F)cc2)N2Cc3ccc(C(F)(F)F)cc3C2=O)cc1]

Synthesized according to General Procedure D using 1-(4-isopropoxyphenyl)-2-(4-(trifluoromethyl)phenoxy)ethan-1-amine (1 eq., 0.442 mmol, 150 mg), methyl 2-(bromomethyl)-5-(trifluoromethyl)benzoate (1.2 eq., 0.530 mmol, 158 mg), DIPEA (2.5 eq., 1.11 mmol, 193 μL), and EtOH (4 mL). Yield: 124 mg (0.237 mmol, 53%). ¹H NMR (500 MHz, CDCl₃) δ 1.33 (dd, J=6.0, 3.6 Hz, 6H), 4.33 (d, J=17.5 Hz, 1H), 4.51-4.61 (m, 3H), 4.71 (dd, J=9.8, 6.9 Hz, 1H), 5.89 (t, J=5.9 Hz, 1H), 6.90 (d, J=8.2 Hz, 2H), 7.00 (d, J=8.7 Hz, 2H), 7.35 (d, J=8.6, 2H), 7.51-7.55 (m, 3H), 7.77 (d, J=8.0 Hz, 1H), 8.15 (s, 1H). ¹³C NMR (125 MHz, CDCl₃) δ 22.1, 48.0, 53.8, 68.0, 70.1, 114.8, 116.3, 121.3 (q, J_{CF}=1.8 Hz), 123.6, 123.8(6), 123.9(4) (q, J_{CF}=272 Hz), 124.4 (q, J_{CF}=271 Hz), 127.1 (q, J_{CF}=3.7 Hz), 128.1, 128.4 (q, J_{CF}=3.5 Hz), 129.2, 131.1 (q, J_{CF}=33 Hz), 133.4, 145.0, 158.1, 160.7, 167.4. HRMS (APCI) m/z calculated for C₂₇H₂₄F₆NO₃ [M+H]⁺ 524.16549, found 524.16480.

### Example 48. Synthesis and Characterization of 6-chloro-2-(1-(4-isopropoxyphenyl)-2-(4-(trifluoromethyl)phenoxy)ethyl)isoindolin-1-one (EU1180-573)

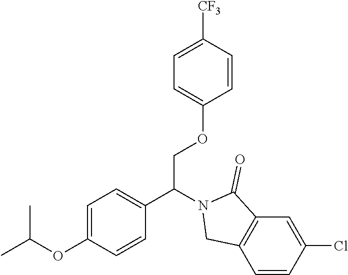 [SMILES: CC(C)Oc1ccc(C(COc2ccc(C(F)(F)F)cc2)N2Cc3ccc(Cl)cc3C2=O)cc1]

Synthesized according to General Procedure D using 1-(4-isopropoxyphenyl)-2-(4-(trifluoromethyl)phenoxy)ethan-1-amine (1 eq., 0.413 mmol, 140 mg), methyl 2-(bromomethyl)-5-chlorobenzoate (1.2 eq., 0.495 mmol, 130 mg), DIPEA (2.5 eq., 1.03 mmol, 180 μL), and EtOH (4 mL). Yield: 78 mg (0.159 mmol, 38%). ¹H NMR (400 MHz, CDCl₃) δ 1.33 (dd, J=6.0, 2.6 Hz, 6H), 4.22 (d, J=17.2 Hz, 1H), 4.47 (d, J=17.1 Hz, 1H), 4.50-4.59 (m, 2H), 4.68 (dd, J=9.9, 6.8 Hz, 1H), 5.85 (t, J=5.9 Hz, 1H), 6.88 (d, J=8.8 Hz, 2H), 7.00 (d, J=8.6 Hz, 2H), 7.30-7.35 (m, 3H), 7.47 (dd, J=8.0, 2.0 Hz, 1H), 7.53 (d, J=8.6 Hz, 1H), 7.84 (d, J=1.9 Hz, 1H). ¹³C NMR (100 MHz, CDCl₃) δ 22.1, 47.6, 53.6, 67.9, 70.0, 114.7, 116.1, 123.6 (q, J_{CF}=33 Hz), 124.0, 124.2, 124.4 (q, J_{CF}=271 Hz), 127.1 (q, J_{CF}=3.7 Hz), 128.2, 129.2, 131.7, 134.3, 134.4, 139.8, 158.0, 160.7, 167.5. HRMS (APCI) m/z calculated for C₂₆H₂₄ClF₃NO₃ [M+H]⁺ 490.13913, found 490.13825.

### Example 49. Synthesis and Characterization of 5-chloro-2-(1-(4-isopropoxyphenyl)-2-(4-(trifluoromethyl)phenoxy)ethyl)isoindolin-1-one (EU1180-574)

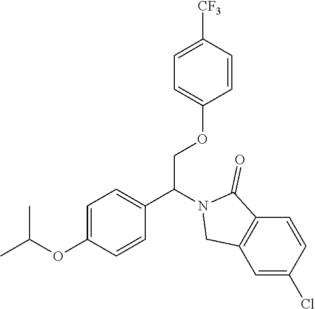 [SMILES: CC(C)Oc1ccc(C(COc2ccc(C(F)(F)F)cc2)N2Cc3cc(Cl)ccc3C2=O)cc1]

Synthesized according to General Procedure D using 1-(4-isopropoxyphenyl)-2-(4-(trifluoromethyl)phenoxy)ethan-1-amine (1 eq., 0.413 mmol, 140 mg), methyl-2-(bromomethyl)-4-chlorobenzoate (1.2 eq., 0.495 mmol, 130 mg), DIPEA (2.5 eq., 1.03 mmol, 180 μL), and EtOH (4 mL). Yield: 120 mg (0.245 mmol, 59%). ¹H NMR (400 MHz, CDCl₃) δ 1.33 (dd, J=6.1, 2.7 Hz, 6H), 4.23 (d, J=17.2 Hz, 1H), 4.45-4.58 (m, 3H), 4.68 (dd, J=9.9, 6.7 Hz, 1H), 5.85 (t, J=5.9 Hz, 1H), 6.88 (d, J=8.8 Hz, 2H), 7.00 (d, J=8.5 Hz, 2H), 7.31-7.36 (m, 3H), 7.42 (dd, J=8.1, 1.8 Hz, 1H), 7.53 (d, J=8.5 Hz, 2H), 7.78 (d, J=8.4 Hz, 1H). ¹³C NMR (100 MHz, CDCl₃) δ 22.1, 47.5, 53.5, 67.9, 69.9, 114.7, 116.1, 123.3, 123.5 (q, J_{CF}=33 Hz), 124.4 (q, J_{CF}=271 Hz), 125.1, 127.1 (q, J_{CF}=3.5 Hz), 128.2, 128.7, 129.2, 131.0, 137.9, 143.2, 157.9, 160.7, 167.8. HRMS (APCI) m/z calculated for C₂₆H₂₄ClF₃NO₃ [M+H]⁺ 490.13913, found 490.13835.

### Example 50. Synthesis and Characterization of 6-fluoro-2-(1-(4-isopropoxyphenyl)-2-(4-(trifluoromethyl)phenoxy)ethyl)isoindolin-1-one (EU1180-575)

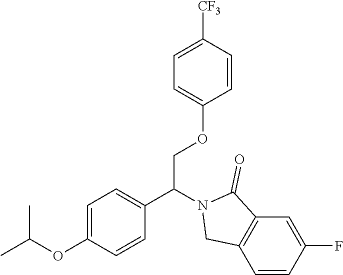 [SMILES: CC(C)Oc1ccc(C(COc2ccc(C(F)(F)F)cc2)N2Cc3ccc(F)cc3C2=O)cc1]

Synthesized according to General Procedure D using 1-(4-isopropoxyphenyl)-2-(4-(trifluoromethyl)phenoxy)ethan-1-amine (1 eq., 0.413 mmol, 140 mg), methyl 2-(bromomethyl)-5-fluorobenzoate (1.2 eq., 0.495 mmol, 122 mg), DIPEA (2.5 eq., 1.03 mmol, 180 μL), and EtOH (4 mL). Yield: 81 mg (0.171 mmol, 42%). ¹H NMR (400 MHz, CDCl₃) δ 1.33 (dd, J=6.0, 2.6 Hz, 6H), 4.22 (d, J=16.8 Hz, 1H), 4.47 (d, J=16.8 Hz, 1H), 4.50-4.59 (m, 2H), 4.68 (dd, J=9.9, 6.8 Hz, 1H), 5.85 (t, J=6.0 Hz, 1H), 6.89 (d, J=8.7 Hz, 2H), 7.00 (d, J=8.9 Hz, 2H), 7.22 (td, J=8.6, 2.5 Hz, 1H), 7.31-7.36 (m, 3H), 7.51-7.56 (m, 3H). ¹³C NMR (100 MHz, CDCl₃) δ 22.1, 47.6, 53.7, 67.9, 70.0, 110.7 (d, J_{CF}=23.5 Hz), 114.7, 116.1, 119.2 (d, J_{CF}=23.7 Hz), 123.6 (q, J_{CF}=33 Hz), 124.3(86) (d, J_{CF}=8.3 Hz), 124.3(97) (q, J_{CF}=271 Hz), 127.1 (q, J_{CF}=3.7 Hz), 128.2, 129.2, 134.6 (d, J_{CF}=8.4 Hz), 137.0 (d, J_{CF}=2.6 Hz), 157.9, 160.7, 162.9 (d, J_{CF}=247 Hz), 167.8 (d, J_{CF}=3.6 Hz). HRMS (APCI) m/z calculated for C₂₆H₂₄F₄NO₃ [M+H]⁺ 474.16868, found 474.16791.

### Example 51. Synthesis and Characterization of 5-fluoro-2-(1-(4-isopropoxyphenyl)-2-(4-(trifluoromethyl)phenoxy)ethyl)isoindolin-1-one (EU1180-576)

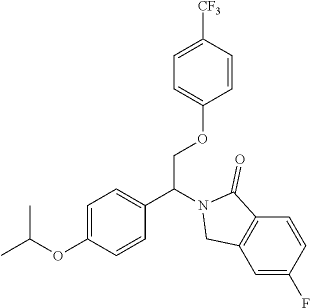 [SMILES: CC(C)Oc1ccc(C(COc2ccc(C(F)(F)F)cc2)N2Cc3cc(F)ccc3C2=O)cc1]

Synthesized according to General Procedure D using 1-(isopropoxyphenyl)-2-(4-(trifluoromethyl)phenoxy)ethan-1-amine (1 eq., 0.413 mmol, 140 mg), methyl 2-(bromomethyl)-4-fluorobenzoate (1.2 eq., 0.495 mmol, 122 mg), DIPEA (2.5 eq., 1.03 mmol, 180 μL), and EtOH (4 mL). Yield: 136 mg (0.287 mmol, 70%). ¹H NMR (400 MHz, CDCl₃) δ 1.31 (dd, J=6.1, 2.6 Hz, 6H), 4.21 (d, J=17.2 Hz, 1H), 4.43-4.57 (m, 3H), 4.66 (dd, J=9.9, 6.6 Hz, 1H), 5.83 (t, J=5.9 Hz, 1H), 6.87 (d, J=8.7 Hz, 2H), 6.98 (d, J=8.8 Hz, 2H), 7.04 (dd, J=8.1, 2.1 Hz, 1H), 7.13 (td, J=8.8, 2.2 Hz, 1H), 7.32 (d, J=8.8, 2H), 7.51 (d, J=9.0, 2H), 7.83 (dd, J=8.4, 5.0 Hz, 1H). ¹³C NMR (100 MHz, CDCl₃) δ 22.0, 47.7, 53.5, 68.0, 70.0, 110.3 (d, J_{CF}=24.3 Hz), 114.7, 116.0 (d, J_{CF}=23.6 Hz), 116.1, 123.6 (q, J_{CF}=33 Hz), 124.4 (q, J_{CF}=271 Hz), 125.9 (d, J_{CF}=9.7 Hz), 127.1 (q, J_{CF}=3.7 Hz), 128.3, 128.6 (d, J=1.9 Hz), 129.2, 144.0 (d, J_{CF}=10.0 Hz), 157.9, 160.7, 165.1 (d, J_{CF}=251 Hz), 167.8. HRMS (APCI) m/z calculated for C₂₆H₂₄F₄NO₃ [M+H]⁺ 474.16868, found 474.16790.

### Example 52. Synthesis and Characterization of 6-bromo-2-(1-(4-isopropoxyphenyl)-2-(4-(trifluoromethyl)phenoxy)ethyl)isoindolin-1-one (EU1180-577)

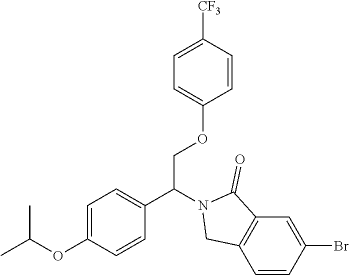 [SMILES: CC(C)Oc1ccc(C(COc2ccc(C(F)(F)F)cc2)N2Cc3ccc(Br)cc3C2=O)cc1]

Synthesized according to General Procedure D using 1-(4-isopropoxyphenyl)-2-(4-(trifluoromethyl)phenoxy)ethan-1-amine (1 eq., 0.413 mmol, 140 mg), methyl 2-(bromomethyl)-5-bromobenzoate (1.2 eq., 0.495 mmol, 153 mg), DIPEA (2.5 eq., 1.03 mmol, 180 μL), and EtOH (4 mL). Yield: 114 mg (0.213 mmol, 52%). ¹H NMR (400 MHz, CDCl₃) δ 1.33 (dd, J=6.1, 2.6 Hz, 6H), 4.20 (d, J=17.2 Hz, 1H), 4.45 (d, J=17.2 Hz, 1H), 4.50-4.59 (m, 2H), 4.67 (dd, J=9.9, 6.8 Hz, 1H), 5.85 (t, J=5.9 Hz, 1H), 6.88 (d, J=8.8 Hz, 2H), 7.00 (d, J=8.6 Hz, 2H), 7.26 (d, J=7.7 Hz, 1H), 7.33 (d, J=8.7 Hz, 2H), 7.53 (d, J=8.6 Hz, 2H), 7.61 (dd, J=8.0, 1.9 Hz, 1H), 7.99 (d, J=1.8 Hz, 1H). ¹³C NMR (100 MHz, CDCl₃) δ 22.1, 47.6, 53.6, 67.9, 70.0, 114.7, 116.1, 122.2, 123.6 (q, J_{CF}=33 Hz), 124.4 (q, J_{CF}=271 Hz), 124.5, 127.1 (q, J_{CF}=3.5 Hz), 128.2, 128.7, 129.2, 134.5, 134.6, 140.2, 157.9, 160.7, 167.3. HRMS (APCI) m/z calculated for C₂₆H₂₄BrF₃NO₃ [M+H]⁺ 534.08862, found 534.08799.

### Example 53. Synthesis and Characterization of 5-bromo-2-(1-(4-isopropoxyphenyl)-2-(4-(trifluoromethyl)phenoxy)ethyl)isoindolin-1-one (EU1180-578)

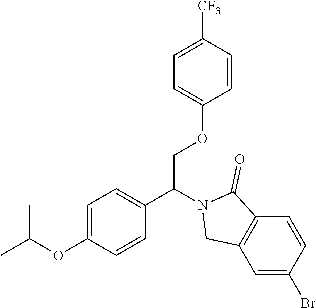 [SMILES: CC(C)Oc1ccc(C(COc2ccc(C(F)(F)F)cc2)N2Cc3cc(Br)ccc3C2=O)cc1]

Synthesized according to General Procedure D using 1-(4-isopropoxyphenyl)-2-(4-(trifluoromethyl)phenoxy)ethan-1-amine (1 eq., 0.413 mmol, 140 mg), methyl 2-(bromomethyl)-4-bromobenzoate (1.2 eq., 0.495 mmol, 153 mg), DIPEA (2.5 eq., 1.03 mmol, 180 μL), and EtOH (4 mL). Yield: 101 mg (0.189 mmol, 46%). ¹H NMR (600 MHz, CDCl₃) δ 1.33 (t, J=5.0 Hz, 6H), 4.22 (d, J=17.1 Hz, 1H), 4.47 (d, J=17.1 Hz, 1H), 4.50-4.57 (m, 2H), 4.68 (dd, J=9.8, 6.8 Hz, 1H), 5.84 (t, J=5.9 Hz, 1H), 6.88 (d, J=8.5 Hz, 2H), 7.00 (d, J=8.5 Hz, 2H), 7.33 (d, J=8.5 Hz, 2H), 7.33 (d, J=8.7 Hz, 2H), 7.51-7.55 (m, 3H), 7.58 (d, J=8.1 Hz, 1H), 7.73 (d, J=8.1 Hz, 1H). ¹³C NMR (150 MHz, CDCl₃) δ 22.1, 47.5, 53.6, 68.0, 70.0, 114.8, 116.2, 123.7 (q, J_{CF}=33 Hz), 124.4 (q, J_{CF}=271 Hz), 125.3, 126.2(7), 126.3(0), 127.1 (q, J_{CF}=3.6 Hz), 128.2, 129.2, 131.5(5), 131.6(1), 143.4, 158.0, 160.7, 167.9. HRMS (APCI) m/z calculated for C₂₆H₂₄BrF₃NO₃ [M+H]⁺ 534.08862 found 534.08788.

### Example 54. Synthesis and Characterization of Compounds from EU1180-516 to EU1180-552

The synthetic route of the foregoing compounds included (1) synthesis of various acetophenone derivatives (Scheme 7), (2) conversion of the acetophenone derivatives to their respective benzylic amines via a reductive amination reaction (Scheme 8), and (3) performing a ring-closing reaction using the benzylic amines with various halogenated and non-halogenated methyl 2-(bromomethyl)benzoate derivatives to furnish the desired isoindolinone ring (Scheme 9).

Scheme 7 illustrates the synthesis of various substituted acetophenone derivatives. First, the acetophenone derivatives underwent radical bromination to afford the 2-bromoacetophenone intermediates in high yield. Second, a Williamson etherification step with 4-hydroxyanisole produced the substituted acetophenone derivatives in high yields. See Lindsay, et al., Organic & Biomolecular Chemistry, 2019, 17, 7408-7415.

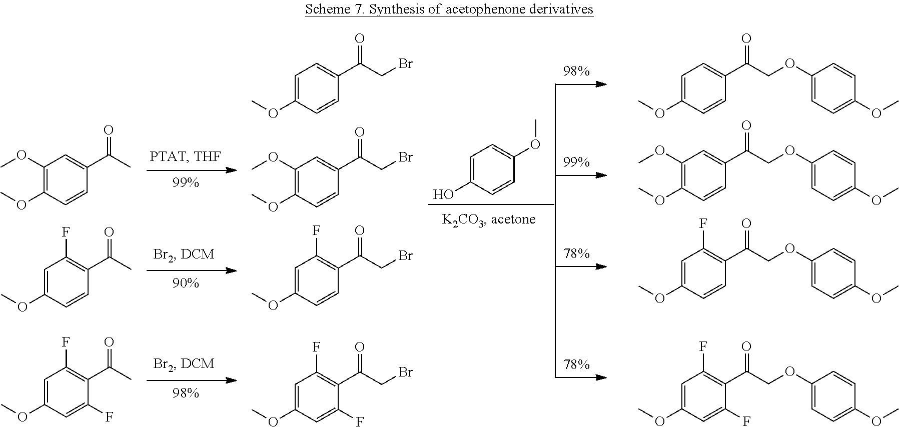

Scheme 8 illustrates the reductive amination step of the acetophenone derivatives. Reductive amination was carried out in one pot with ammonium acetate and sodium cyanoborohydride in methanol to afford the advanced benzylic secondary amine intermediates.

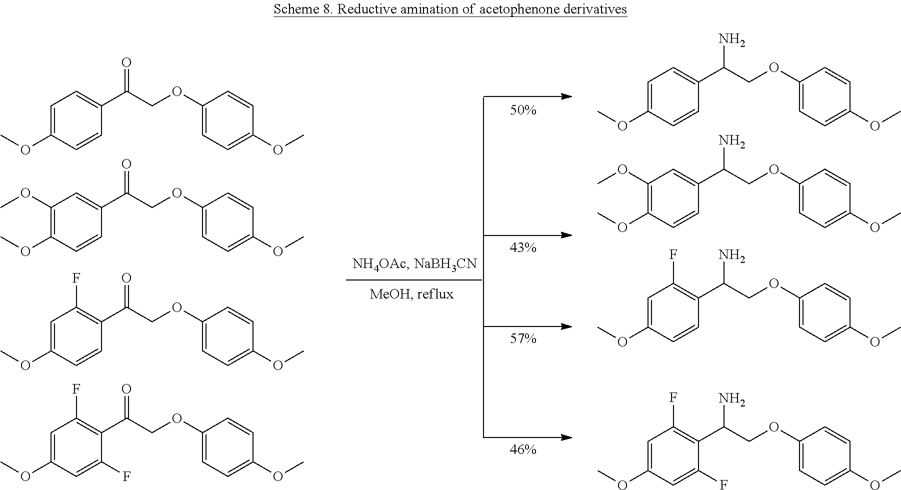

Scheme 9 illustrates the ring-closing step performed using the benzylic secondary amine derivatives from Scheme 8 and the substituted (or unsubstituted) methyl (2-bromomethyl)benzoates. See General Procedure G for more details.

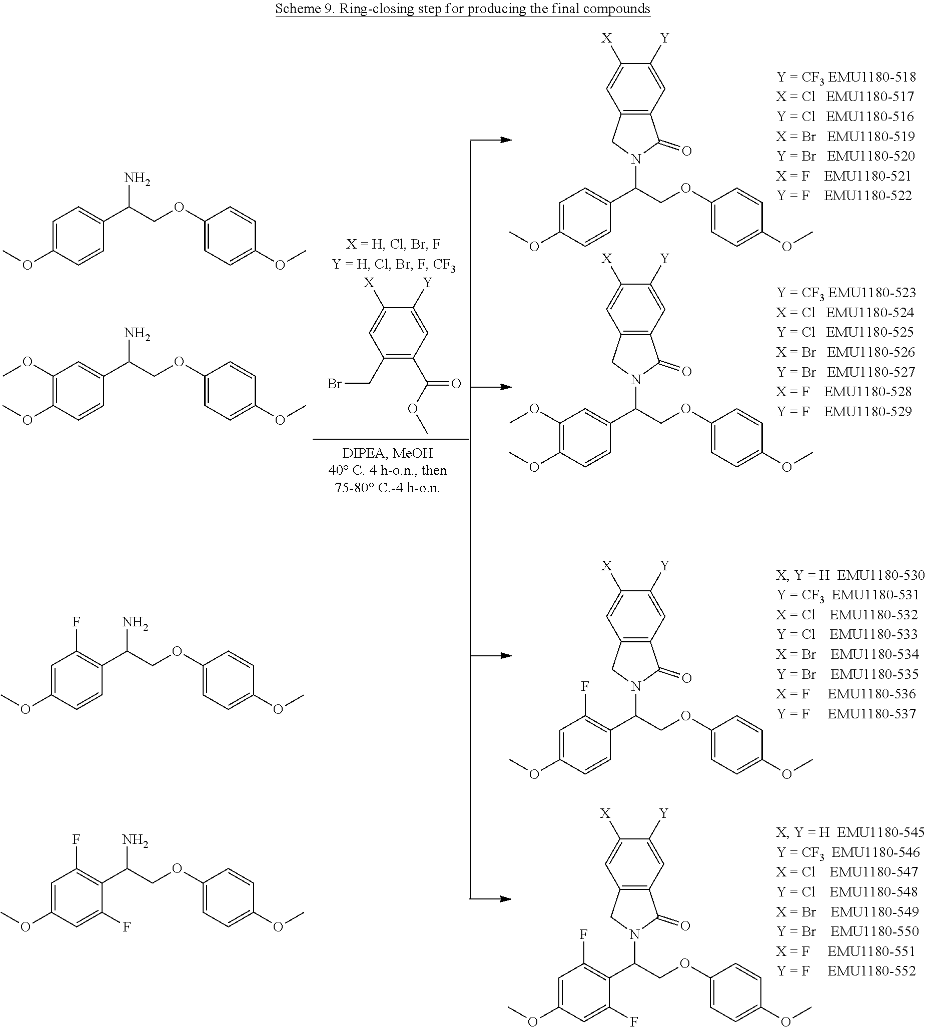

6-Chloro-2-(2-(4-methoxyphenoxy)-1-(4-methoxyphenyl)ethyl)isoindolin-1l-one (EU1180-516) was synthesized using General Procedure G with a yield of 78%. ¹H NMR (500 MHz, CDCl₃) δ 7.85 (d, J=2.0 Hz, 1H), 7.46 (dd, J=8.1, 2.0 Hz, 1H), 7.38-7.33 (m, 2H), 7.30 (d, J=8.1 Hz, 1H), 6.91-6.84 (m, 4H), 6.84-6.79 (m, 2H), 5.86-5.80 (m, 1H), 4.57 (dd, J=10.0, 6.5 Hz, 1H), 4.52 (d, J=17.3 Hz, 1H), 4.46 (dd, J=10.0, 5.0 Hz, 1H), 4.21 (d, J=16.9 Hz, 1H), 3.79 (s, 3H), 3.75 (s, 3H). ¹³C NMR (126 MHz, CDCl₃) δ 167.5, 159.5, 154.5, 152.5, 139.9, 134.5, 134.4, 131.6, 129.3, 129.2, 124.1, 124.1, 115.9, 114.9, 114.4, 68.8, 55.8, 55.4, 53.7, 47.7. HRMS-ESI+: m/z [M+H]⁺ calculated for C₂₄H₂₃O₄N³⁵Cl: 424.1310; found: 424.1303.

5-Chloro-2-(2-(4-methoxyphenoxy)-1-(4-methoxyphenyl)ethyl)isoindolin-1-one (EU1180-517) was synthesized using General Procedure G with a yield of 55%. ¹H NMR (500 MHz, CDCl₃) δ 7.79 (d, J=8.1 Hz, 1H), 7.42 (dd, J=8.1, 1.8 Hz, 1H), 7.38-7.33 (m, 3H), 6.91-6.80 (m, 6H), 5.86-5.81 (m, 1H), 4.57 (dd, J=10.0, 6.5 Hz, 1H), 4.53 (d, J=17.3 Hz, 1H), 4.46 (dd, J=10.0, 5.0 Hz, 1H), 4.21 (d, J=17.2 Hz, 1H), 3.79 (s, 3H), 3.75 (s, 3H). ¹³C NMR (126 MHz, CDCl₃) δ 167.7, 159.4, 154.4, 152.5, 143.4, 137.8, 131.2, 129.2, 129.2, 128.7, 125.1, 123.3, 115.9, 114.8, 114.3, 68.8, 55.8, 55.4, 53.6, 47.6. HRMS-ESI+: m/z [M+H]⁺ calculated for C₂₄H₂₂O₄N³⁵Cl: 424.1310; found: 424.1304.

2-(2-(4-Methoxyphenoxy)-1-(4-methoxyphenyl)ethyl)-6-(trifluoromethyl)isoindolin-1-one (EU1180-518) was synthesized using General Procedure G with a yield of 88%. ¹H NMR (500 MHz, CDCl₃) δ 8.16 (s, 1H), 7.76 (ddd, J=8.0, 1.7, 0.8 Hz, 1H), 7.51 (d, J=7.9 Hz, 1H), 7.40-7.34 (m, 2H), 6.91-6.84 (m, 4H), 6.84-6.79 (m, 2H), 5.87 (dd, J=6.5, 4.9 Hz, 1H), 4.63 (d, J=17.6 Hz, 1H), 4.59 (dd, J=10.1, 6.6 Hz, 1H), 4.48 (dd, J=10.1, 5.0 Hz, 1H), 4.31 (d, J=17.6 Hz, 1H), 3.79 (s, 3H), 3.75 (s, 3H). ¹³C NMR (126 MHz, CDCl₃) δ 167.4, 159.5, 154.5, 152.4, 145.2, 133.5, 131.0 (q, J=33.3, 32.6 Hz), 129.2, 129.1, 128.2 (q, J=3.4 Hz), 124.0 (q, J=272.5 Hz), 123.6, 121.3 (q, J=3.9 Hz), 115.9, 114.9, 114.40, 68.8, 55.8 (d, J=2.0 Hz), 55.4 (d, J=1.8 Hz), 53.7, 48.0. HRMS-ESI+: m/z [M+H]⁺ calculated for C₂₅H₂₃O₄NF₃: 458.1574; found: 458.1566.

5-Bromo-2-(2-(4-methoxyphenoxy)-1-(4-methoxyphenyl)ethyl)isoindolin-1-one (EU1180-519) was synthesized using General Procedure G with a yield of 43%. ¹H NMR (500 MHz, CDCl₃) δ 7.74 (d, J=8.1 Hz, 1H), 7.59 (dd, J=8.1, 1.6 Hz, 1H), 7.54-7.50 (m, 1H), 7.38-7.33 (m, 2H), 6.91-6.80 (m, 6H), 5.83 (t, J=5.7 Hz, 1H), 4.59-4.50 (m, 2H), 4.46 (dd, J=10.0, 5.0 Hz, 1H), 4.21 (d, J=17.3 Hz, 1H), 3.79 (s, 3H), 3.76 (s, 3H). ¹³C NMR (126 MHz, CDCl₃) δ 167.9, 159.5, 154.5, 152.5, 143.6, 131.7, 131.5, 129.2, 129.2, 126.3, 126.2, 125.4, 115.9, 114.9, 114.4, 68.8, 55.9, 55.4, 53.6, 47.6. HRMS-ESI+: m/z [M+H]⁺ calculated for C₂₄H₂₂O₄N⁷⁹Br: 468.0805; found: 468.0804.

6-Bromo-2-(2-(4-methoxyphenoxy)-1-(4-methoxyphenyl)ethyl)isoindolin-1-one (EU1180-520) was synthesized using General Procedure G with a yield of 49%. ¹H NMR (500 MHz, CDCl₃) δ 8.01 (d, J=1.9 Hz, 1H), 7.61 (dd, J=8.0, 1.9 Hz, 1H), 7.39-7.32 (m, 2H), 7.25 (d, J=7.9 Hz, 1H), 6.92-6.79 (m, 6H), 5.83 (t, J=5.8 Hz, 1H), 4.60-4.43 (m, 3H), 4.19 (d, J=17.2 Hz, 1H), 3.79 (s, 3H), 3.76 (s, 3H). ¹³C NMR (126 MHz, CDCl₃) δ 167. 3, 159.5, 154.5, 152.5, 140.4, 134.8, 134.4, 129.2, 129.2, 127.1, 124.46, 122.1, 115.9, 114.9, 114.4, 68.8, 55.8, 55.4, 53.7, 47.8. HRMS-ESI+: m/z [M+H]⁺ calculated for C₂₄H₂₂O₄N⁷⁹Br: 468.0805; found: 468.0801.

5-Fluoro-2-(2-(4-methoxyphenoxy)-1-(4-methoxyphenyl)ethyl)isoindolin-1-one (EU1180-521) was synthesized using General Procedure G with a yield of 40%. ¹H NMR (500 MHz, CDCl₃) δ 7.85 (dd, J=8.4, 5.0 Hz, 1H), 7.39-7.33 (m, 2H), 7.15 (ddd, J=9.2, 8.4, 2.3 Hz, 1H), 7.06 (dd, J=8.1, 2.2 Hz, 1H), 6.91-6.85 (m, 4H), 6.85-6.80 (m, 2H), 5.83 (t, J=5.7 Hz, 1H), 4.61-4.44 (m, 3H), 4.22 (d, J=17.2 Hz, 1H), 3.79 (s, 3H), 3.76 (s, 3H). ¹³C NMR (126 MHz, CDCl₃) δ 167.8, 166.2, 164.2, 159.4, 154.5, 152.5, 144.2 (d, J=10.0 Hz), 129.3, 129.3, 128.78, 125.9 (d, J=9.9 Hz), 115.9, 114.9, 114.3, 113.1 (dd, J=709.2, 23.8 Hz), 68.9, 55.8, 55.4, 53.6, 47.7. HRMS-ESI+: m/z [M+H]⁺ calculated for C₂₄H₂₃O₄NF: 408.1606; found: 408.1600.

6-Fluoro-2-(2-(4-methoxyphenoxy)-1-(4-methoxyphenyl)ethyl)isoindolin-1-one (EU1180-522) was synthesized using General Procedure G with a yield of 58%. ¹H NMR (500 MHz, CDCl₃) δ 7.54 (dd, J=7.7, 2.5 Hz, 1H), 7.39-7.34 (m, 2H), 7.33 (dd, J=8.3, 4.4 Hz, 1H), 7.21 (td, J=8.6, 2.4 Hz, 1H), 6.91-6.85 (m, 4H), 6.85-6.80 (m, 2H), 5.84 (dd, J=6.5, 5.1 Hz, 1H), 4.57 (dd, J=10.0, 6.6 Hz, 1H), 4.52 (d, J=16.9 Hz, 1H), 4.47 (dd, J=10.0, 5.1 Hz, 1H), 4.21 (d, J=16.9 Hz, 1H), 3.79 (s, 3H), 3.75 (s, 3H). ¹³C NMR (126 MHz, CDCl₃) δ 167.7, 163.9, 161.9, 159.5, 154.5, 152.5, 137.2 (d, J=2.5 Hz), 134.8 (d, J=8.4 Hz), 129.2, 124.3 (d, J=8.2 Hz), 119.0 (d, J=23.6 Hz), 115.9, 114.9, 114.3, 110.6 (d, J=23.4 Hz), 68.8, 55.8 (d, J=1.9 Hz), 55.4 (d, J=2.0 Hz), 53.7, 47.6 (d, J=2.2 Hz). HRMS-ESI+: m/z [M+H]⁺ calculated for C₂₄H₂₃O₄NF: 408.1606; found: 408.1600.

2-(1-(3,4-Dimethoxyphenyl)-2-(4-methoxyphenoxy)ethyl)-6-(trifluoromethyl)isoindolin-1-one (EU1180-523) was synthesized using General Procedure G with a yield of 56%. ¹H NMR (500 MHz, CDCl₃) δ 8.16 (s, 1H), 7.77 (ddd, J=7.9, 1.8, 0.7 Hz, 1H), 7.51 (d, J=7.9 Hz, 1H), 7.01 (ddd, J=8.3, 2.1, 0.6 Hz, 1H), 6.97 (d, J=2.1 Hz, 1H), 6.90-6.79 (m, 5H), 5.88-5.81 (m, 1H), 4.64 (d, J=17.6 Hz, 1H), 4.59 (dd, J=10.1, 6.4 Hz, 1H), 4.50 (dd, J=10.1, 4.9 Hz, 1H), 4.30 (d, J=17.6 Hz, 1H), 3.87 (s, 3H), 3.83 (s, 3H), 3.75 (s, 3H). ¹³C NMR (126 MHz, CDCl₃) δ 167.4, 154.5, 152.4, 149.5, 149.1, 145.2, 133.5, 131.2, 130.9, 129.67, 128.4-128.2 (m), 125.0, 123.6, 122.9, 121.4-121.2 (m), 120.0, 116.0, 114.9, 111.8, 111.3, 68.9, 56.2, 56.1, 55.8, 54.0, 48.1. HRMS-ESI+: m/z [M+H]⁺ calculated for C₂₆H₂₅O₅NF₃: 488.1679; found: 488.1677.

5-Chloro-2-(1-(3,4-dimethoxyphenyl)-2-(4-methoxyphenoxy)ethyl)isoindolin-1-one (EU1180-524) was synthesized using General Procedure G with a yield of 50%. ¹H NMR (500 MHz, CDCl₃) δ 7.80 (d, J=8.2 Hz, 1H), 7.45-7.41 (m, 1H), 7.37-7.35 (m, 1H), 7.01-6.98 (m, 1H), 6.95 (d, J=2.1 Hz, 1H), 6.88-6.80 (m, 5H), 5.81 (t, J=5.7 Hz, 1H), 4.59-4.51 (m, 2H), 4.48 (dd, J=10.0, 5.1 Hz, 1H), 4.21 (d, J=17.2 Hz, 1H), 3.87 (s, 3H), 3.83 (s, 3H), 3.75 (s, 3H). ¹³C NMR (126 MHz, CDCl₃) δ 167.8, 154.5, 152.5, 149.4, 149.0, 143.4, 137.9, 131.2, 129.7, 128.7, 125.1, 123.3, 120.0, 115.9, 114.9, 111.7, 111.3, 68.9, 56.2, 56.1, 55.8, 53.9, 47.7. HRMS-ESI+: m/z [M+H]⁺ calculated for C₂₅H₂₅O₅N³⁵Cl: 454.1416; found: 454.1413.

6-Chloro-2-(1-(3,4-dimethoxyphenyl)-2-(4-methoxyphenoxy)ethyl)isoindolin-1-one (EU1180-525) was synthesized using General Procedure G with a yield of 50% yield. ¹H NMR (500 MHz, CDCl₃) δ 7.86-7.84 (m, 1H), 7.48-7.45 (m, 1H), 7.31 (dt, J=8.0, 0.7 Hz, 1H), 7.02-6.98 (m, 1H), 6.96 (d, J=2.1 Hz, 1H), 6.88-6.80 (m, 5H), 5.81 (dd, J=6.3, 5.2 Hz, 1H), 4.59-4.50 (m, 2H), 4.48 (dd, J=10.0, 5.0 Hz, 1H), 4.20 (d, J=17.1 Hz, 1H), 3.87 (s, 3H), 3.83 (s, 3H), 3.76-3.75 (m, 3H). ¹³C NMR (126 MHz, CDCl₃) δ 167.5, 154.5, 152.5, 149.4, 149.1, 139.9, 134.5, 134.4, 131.7, 129.7, 124.1, 124.1, 120.0, 116.0, 114.9, 111.8, 111.3, 68.9, 56.2, 56.1, 55.9, 54.0, 47.8. HRMS-ESI+: m/z [M+H]⁺ calculated for C₂₅H₂₅O₅N³⁵Cl: 454.1416; found: 454.1415.

5-Bromo-2-(1-(3,4-dimethoxyphenyl)-2-(4-methoxyphenoxy)ethyl)isoindolin-1-one (EU1180-526) was synthesized using General Procedure G with a yield of 52%. ¹H NMR (500 MHz, CDCl₃) δ 7.74 (dd, J=8.1, 0.6 Hz, 1H), 7.60-7.57 (m, 1H), 7.54-7.52 (m, 1H), 6.99 (ddd, J=8.3, 2.1, 0.6 Hz, 1H), 6.95 (d, J=2.1 Hz, 1H), 6.88-6.80 (m, 5H), 5.84-5.78 (m, 1H), 4.59-4.51 (m, 2H), 4.47 (dd, J=10.0, 5.1 Hz, 1H), 4.21 (d, J=17.3 Hz, 1H), 3.87 (s, 3H), 3.83 (s, 3H), 3.75 (s, 3H). ¹³C NMR (126 MHz, CDCl₃) δ 167.9, 154.5, 152.5, 149.4, 149.0, 143.6, 131.6, 131.6, 129.7, 126.3, 126.2, 125.4, 120.0, 115.9, 114.9, 111.7, 111.3, 68.9, 56.1, 56.0, 55.8, 53.9, 47.6. HRMS-ESI+: m/z [M+H]⁺ calculated for C₂₅H₂₅O₅N⁷⁹Br: 498.0911; found: 498.0911.

6-Bromo-2-(1-(3,4-dimethoxyphenyl)-2-(4-methoxyphenoxy)ethyl)isoindolin-1-one (EU1180-527) was synthesized using General Procedure G with a yield of 59%. ¹H NMR (500 MHz, CDCl₃) δ 8.02-7.98 (m, 1H), 7.61 (ddd, J=8.0, 1.9, 0.7 Hz, 1H), 7.27-7.23 (m, 1H), 7.02-6.97 (m, 1H), 6.95 (d, J=2.1 Hz, 1H), 6.89-6.80 (m, 5H), 5.84-5.78 (m, 1H), 4.59-4.54 (m, 1H), 4.53-4.45 (m, 2H), 4.18 (d, J=17.2 Hz, 1H), 3.87-3.85 (m, 3H), 3.84-3.81 (m, 3H), 3.75 (d, J=0.7 Hz, 3H). ¹³C NMR (126 MHz, CDCl₃) δ 167.3, 154.5, 152.4, 149.4, 149.0, 140.4, 134.7, 134.5, 129.7, 127.1, 124.5, 122.1, 112.0, 115.9, 114.9, 111.8, 111.3, 68.9, 56.1, 56.0, 55.8, 53.9, 47.8. HRMS-ESI+: m/z [M+H]⁺ calculated for C₂₅H₂₅O₅N⁷⁹Br: 498.0911; found: 498.0909.

2-(1-(3,4-Dimethoxyphenyl)-2-(4-methoxyphenoxy)ethyl)-5-fluoroisoindolin-1-one (EU1180-528) was synthesized using General Procedure G with a yield of 54%. ¹H NMR (500 MHz, CDCl₃) δ 7.85 (dd, J=8.4, 5.0 Hz, 1H), 7.14 (td, J=8.8, 2.3 Hz, 1H), 7.06 (dd, J=8.2, 2.2 Hz, 1H), 7.00 (dd, J=8.3, 2.1 Hz, 1H), 6.96 (d, J=2.1 Hz, 1H), 6.89-6.79 (m, 5H), 5.81 (t, J=5.7 Hz, 1H), 4.58-4.50 (m, 2H), 4.48 (dd, J=10.0, 5.1 Hz, 1H), 4.21 (d, J=17.2 Hz, 1H), 3.86 (s, 3H), 3.83 (s, 3H), 3.75 (s, 3H). ¹³C NMR (126 MHz, CDCl₃) δ 167.8, 165.2 (d, J=251.1 Hz), 154.5, 152.5, 149.4, 149.0, 144.2 (d, J=10.0 Hz), 129.8, 128.7 (d, J=2.0 Hz), 125.9 (d, J=9.7 Hz), 112.0, 115.9, 115.9 (d, J=23.5 Hz), 114.8, 111.8, 111.3, 110.2 (d, J=24.1 Hz), 68.9, 56.1, 56.0, 55.8, 53.8, 47.8. HRMS-ESI+: m/z [M+H]⁺ calculated for C₂₅H₂₅O₅NF: 438.1711; found: 438.1709.

2-(1-(3,4-Dimethoxyphenyl)-2-(4-methoxyphenoxy)ethyl)-6-fluoroisoindolin-1-one (EU1180-529) was synthesized using General Procedure G with a yield of 59%. ¹H NMR (500 MHz, CDCl₃) δ 7.54 (dd, J=7.7, 2.4 Hz, 1H), 7.33 (dd, J=8.3, 4.5 Hz, 1H), 7.21 (td, J=8.6, 2.4 Hz, 1H), 7.01-6.98 (m, 1H), 6.96 (d, J=2.1 Hz, 1H), 6.89-6.80 (m, 5H), 5.82 (dd, J=6.3, 5.2 Hz, 1H), 4.57 (dd, J=10.0, 6.4 Hz, 1H), 4.52 (d, J=16.9 Hz, 1H), 4.48 (dd, J=10.0, 5.1 Hz, 1H), 4.20 (d, J=16.8 Hz, 1H), 3.86 (s, 3H), 3.83 (s, 3H), 3.75-3.74 (m, 3H). ¹³C NMR (126 MHz, CDCl₃) δ 167.8 (d, J=3.4 Hz), 162.9 (d, J=247.0 Hz), 154.5, 152.5, 149.4, 149.0, 137.1 (d, J=2.4 Hz), 134.7 (d, J=8.6 Hz), 129.7, 124.3 (d, J=8.3 Hz), 112.0, 119.1 (d, J=23.7 Hz), 115.9, 114.9, 111.8, 111.3, 110.6 (d, J=23.5 Hz), 68.8, 56.1, 56.0, 55.8, 54.0, 47.7. HRMS-ESI+: m/z [M+H]⁺ calculated for C₂₅H₂₅O₅NF: 438.1711; found: 438.1708.

2-(1-(2-Fluoro-4-methoxyphenyl)-2-(4-methoxyphenoxy)ethyl)isoindolin-1-one (EU1180-530) was synthesized using General Procedure G with a yield of 42%. ¹H NMR (500 MHz, CDCl₃) δ 7.86 (d, J=7.6 Hz, 1H), 7.53-7.41 (m, 3H), 7.39 (d, J=7.5 Hz, 1H), 6.88-6.84 (m, 2H), 6.83-6.79 (m, 2H), 6.70 (dd, J=8.7, 2.6 Hz, 1H), 6.63 (dd, J=12.3, 2.5 Hz, 1H), 5.94 (dd, J=7.2, 5.3 Hz, 1H), 4.67-4.62 (m, 1H), 4.60 (d, J=16.9 Hz, 1H), 4.47 (dd, J=10.0, 5.3 Hz, 1H), 4.33 (d, J=16.9 Hz, 1H), 3.78 (s, 3H), 3.75 (s, 3H) ¹³C NMR (126 MHz, CDCl₃) δ 168.6, 161.7 (d, J=247.1 Hz), 161.0 (d, J=11.1 Hz), 154.4, 152.5, 141.8, 132.7, 131.47, 130.7 (d, J=6.2 Hz), 128.1, 123.9, 122.8, 116.4 (d, J=15.1 Hz), 116.0, 114.8, 110.2 (d, J=3.0 Hz), 102.1 (d, J=26.0 Hz), 68.3, 50.2, 48.6. HRMS-ESI+: m/z [M+H]⁺ calculated for C₂₄H₂₃O₄NF: 408.1606; found: 408.1600.

2-(1-(2-Fluoro-4-methoxyphenyl)-2-(4-methoxyphenoxy)ethyl)-6-(trifluoromethyl)isoindolin-1-one (EU1180-531) was synthesized using General Procedure G with a yield of 70%. ¹H NMR (500 MHz, CDCl₃) δ 8.13 (s, 1H), 7.78-7.75 (m, 1H), 7.54-7.47 (m, 2H), 6.87-6.83 (m, 2H), 6.83-6.79 (m, 2H), 6.73-6.69 (m, 1H), 6.64 (dd, J=12.4, 2.6 Hz, 1H), 5.93 (dd, J=7.2, 5.0 Hz, 1H), 4.71-4.64 (m, 2H), 4.47 (dd, J=10.1, 5.0 Hz, 1H), 4.39 (d, J=17.5 Hz, 1H), 3.79 (s, 3H), 3.75 (s, 3H). ¹³C NMR (126 MHz, CDCl₃) δ 167.2, 161.9 (d, J=187.6 Hz), 160.9 (d, J=48.0 Hz), 154.5, 152.3, 145.2, 133.5, 130.72 (d, J=6.2 Hz), 128.3, 125.1, 123.5, 122.9, 121.3, 116.0 (d, J=14.5 Hz), 116.0, 114.9, 110.3 (d, J=2.8 Hz), 102.2 (d, J=26.0 Hz), 68.2, 55.8, 55.7, 50.4, 48.7. HRMS-ESI+: m/z [M+H]⁺ calculated for C₂H₂₂O₄NF₄: 476.1480; found: 476.1470.

5-Chloro-2-(1-(2-fluoro-4-methoxyphenyl)-2-(4-methoxyphenoxy)ethyl)isoindolin-1-one (EU1180-532) was synthesized using General Procedure G with a yield of 53%. ¹H NMR (500 MHz, CDCl₃) δ 7.77 (d, J=8.1 Hz, 1H), 7.48 (t, J=8.6 Hz, 1H), 7.44-7.39 (m, 1H), 7.39-7.34 (m, 1H), 6.88-6.78 (m, 4H), 6.70 (dd, J=8.6, 2.6 Hz, 1H), 6.63 (dd, J=12.3, 2.6 Hz, 1H), 5.90 (dd, J=7.1, 5.1 Hz, 1H), 4.63 (dd, J=10.1, 6.9 Hz, 1H), 4.58 (d, J=17.2 Hz, 1H), 4.45 (dd, J=10.1, 5.1 Hz, 1H), 4.30 (d, J=17.2 Hz, 1H), 3.78 (s, 3H), 3.75 (s, 3H). ¹³C NMR (126 MHz, CDCl₃) δ 167.6, 162.7, 161.1, 161.0, 160.7, 154.5, 152.4, 143.4, 137.8, 131.2, 130.7, 130.7, 128.7, 125.1, 123.3, 119.3, 116.19, 116.1, 116.0, 114.8, 114.6, 114.5, 110.3 (d, J=2.9 Hz), 102.3, 102.1, 55.8, 55.7, 50.3, 48.3. HRMS-ESI+: m/z [M+H]⁺ calculated for C₂₄H₂₂O₄N³⁵ClF: 442.1216; found: 442.1206.

6-Chloro-2-(1-(2-fluoro-4-methoxyphenyl)-2-(4-methoxyphenoxy)ethyl)isoindolin-1-one (EU1180-533) was synthesized using General Procedure G with a yield of 48%. ¹H NMR (500 MHz, CDCl₃) δ 7.82 (d, J=1.9 Hz, 1H), 7.50-7.45 (m, 2H), 7.32 (d, J=8.1 Hz, 1H), 6.87-6.79 (m, 4H), 6.70 (dd, J=8.6, 2.6 Hz, 1H), 6.63 (dd, J=12.3, 2.6 Hz, 1H), 5.90 (dd, J=7.2, 5.1 Hz, 1H), 4.66-4.61 (m, 1H), 4.58 (d, J=17.1 Hz, 1H), 4.45 (dd, J=10.1, 5.2 Hz, 1H), 4.30 (d, J=17.1 Hz, 1H), 3.79 (s, 3H), 3.75 (s, 3H). ¹³C NMR (126 MHz, CDCl₃) δ 167.3, 162.7, 161.1, 161.0, 160.7, 154.5, 152.4, 139.9, 134.5, 134.4, 131.7, 130.7, 130.7, 124.1, 124.1, 124.1, 116.2, 116.1, 116.0, 114.9, 114.5, 110.3, 110.3, 102.3, 102.1, 68.2, 55.8, 55.7, 50.4, 48.4. HRMS-ESI+: m/z [M+H]⁺ calculated for C₂₄H₂₂O₄N³⁵ClF: 442.1216; found: 442.1209.

5-Bromo-2-(1-(2-fluoro-4-methoxyphenyl)-2-(4-methoxyphenoxy)ethyl)isoindolin-1-one (EU1180-534) was synthesized using General Procedure G with a yield of 49%. ¹H NMR (500 MHz, CDCl₃) δ 7.71 (d, J=8.0 Hz, 1H), 7.59-7.56 (m, 1H), 7.55-7.54 (m, 1H), 7.48 (t, J=8.6 Hz, 1H), 6.87-6.80 (m, 4H), 6.70 (dd, J=8.6, 2.6 Hz, 1H), 6.65-6.61 (m, 1H), 5.89 (dd, J=7.2, 5.1 Hz, 1H), 4.63 (dd, J=9.8, 7.4 Hz, 1H), 4.58 (d, J=17.2 Hz, 1H), 4.45 (dd, J=10.1, 5.1 Hz, 1H), 4.30 (d, J=17.2 Hz, 1H), 3.79 (s, 4H), 3.75 (s, 4H). ¹³C NMR (126 MHz, CDCl₃) δ 167.7, 162.7, 161.1, 161.0, 160.7, 154.46, 152.4, 143.6, 131.7, 131.5, 130.7, 130.7, 126.3, 126.2, 125.3, 119.3, 116.2, 116.0, 116.0, 114.9, 114.6, 114.5, 110.3, 110.3, 102.3, 102.1, 68.2, 55.9, 55.8, 50.3. HRMS-ESI+: m/z [M+H]⁺ calculated for C₂₄H₂₂O₄N⁷⁹BrF: 486.0711; found: 486.0697.

6-Bromo-2-(1-(2-fluoro-4-methoxyphenyl)-2-(4-methoxyphenoxy)ethyl)isoindolin-1-one (EU1180-535) was synthesized using General Procedure G with a yield of 28%. ¹H NMR (500 MHz, CDCl₃) δ 7.98 (d, J=1.9 Hz, 1H), 7.62 (dd, J=8.1, 1.8 Hz, 1H), 7.47 (t, J=8.6 Hz, 1H), 7.27 (d, J=8.1 Hz, 1H), 6.86-6.83 (m, 2H), 6.83-6.79 (m, 2H), 6.70 (dd, J=8.7, 2.6 Hz, 1H), 6.63 (dd, J=12.4, 2.6 Hz, 1H), 5.90 (dd, J=7.2, 5.1 Hz, 1H), 4.65-4.61 (m, 1H), 4.55 (d, J=17.1 Hz, 1H), 4.45 (dd, J=10.1, 5.1 Hz, 1H), 4.28 (d, J=17.1 Hz, 1H), 3.79 (s, 3H), 3.75 (s, 3H). ¹³C NMR (126 MHz, CDCl₃) δ 167.2, 162.7, 161.0, 154.5, 152.4, 140.4, 134.8, 134.5, 130.8, 130.7, 127.1, 124.4, 122.2, 116.2, 116.1, 116.0, 114.9, 110.3, 110.3, 102.3, 102.1, 68.2, 55.9, 55.8, 50.4. HRMS-ESI+: m/z [M+H]⁺ calculated for C₂₄H₂₂O₄N⁷⁹BrF: 486.0701; found: 486.0711.

5-Fluoro-2-(1-(2-fluoro-4-methoxyphenyl)-2-(4-methoxyphenoxy)ethyl)isoindolin-1-one (EU1180-536) was synthesized using General Procedure G with a yield of 65%. ¹H NMR (500 MHz, CDCl₃) δ 7.82 (dd, J=8.4, 5.0 Hz, 1H), 7.48 (t, J=8.6 Hz, 1H), 7.14 (td, J=8.8, 2.3 Hz, 1H), 7.07 (dd, J=8.2, 2.2 Hz, 1H), 6.88-6.79 (m, 4H), 6.70 (dd, J=8.7, 2.6 Hz, 1H), 6.63 (dd, J=12.4, 2.6 Hz, 1H), 5.90 (dd, J=7.1, 5.2 Hz, 1H), 4.63 (dd, J=10.0, 7.1 Hz, 1H), 4.58 (d, J=17.2 Hz, 1H), 4.46 (dd, J=10.1, 5.2 Hz, 1H), 4.30 (d, J=17.2 Hz, 1H), 3.78 (s, 3H), 3.75 (s, 3H). ¹³C NMR (126 MHz, CDCl₃) δ 167.58, 166.16, 164.17, 162.67, 161.06, 160.97, 160.70, 154.44, 152.40, 144.17, 144.09, 130.74, 130.69, 128.76, 128.75, 125.92, 125.84, 119.32, 116.29, 116.17, 115.95, 115.77, 114.83, 114.55, 114.49, 110.30, 110.26, 110.23, 110.11, 102.27, 102.06, 68.26, 55.82, 55.72, 50.25, 48.37. HRMS-ESI+: m/z [M+H]⁺ calculated for C₂₄H₂₂O₄NF₂: 426.1511; found: 426.1504.

6-Fluoro-2-(1-(2-fluoro-4-methoxyphenyl)-2-(4-methoxyphenoxy)ethyl)isoindolin-1-one (EU1180-537) was synthesized using General Procedure G with a yield of 70%. ¹H NMR (500 MHz, CDCl₃) δ 7.51 (dd, J=7.6, 2.5 Hz, 1H), 7.48 (t, J=8.6 Hz, 1H), 7.35 (dd, J=8.3, 4.4 Hz, 1H), 7.21 (td, J=8.7, 2.5 Hz, 1H), 6.88-6.79 (m, 4H), 6.70 (dd, J=8.7, 2.7 Hz, 1H), 6.63 (dd, J=12.3, 2.6 Hz, 1H), 5.91 (dd, J=7.2, 5.2 Hz, 1H), 4.64 (dd, J=10.1, 7.2 Hz, 1H), 4.57 (d, J=16.8 Hz, 1H), 4.46 (dd, J=10.1, 5.2 Hz, 1H), 4.30 (d, J=16.8 Hz, 1H), 3.78 (s, 3H), 3.75 (s, 3H). ¹³C NMR (126 MHz, CDCl₃) δ 167.6, 167.6, 163.9, 162.7, 161.9, 161.1, 161.0, 161.0, 154.4, 152.4, 137.1, 137.1, 134.8, 134.7, 130.7, 130.7, 124.3, 124.3, 119.1, 118.9, 116.2, 116.1, 116.0, 114.8, 110.7, 110.5, 110.3, 110.2, 102.3, 102.1, 55.8, 55.7, 50.4, 48.3. HRMS-ESI+: m/z [M+H]⁺ calculated for C₂₄H₂₂O₄NF₂: 426.1511; found: 426.1503.

2-(1-(2,6-Difluoro-4-methoxyphenyl)-2-(4-methoxyphenoxy)ethyl)isoindolin-1-one (EU1180-545) was synthesized using General Procedure G with a yield of 52%. ¹H NMR (500 MHz, CDCl₃) δ 7.85 (dd, J=7.5, 1.0 Hz, 1H), 7.51 (td, J=7.5, 1.2 Hz, 1H), 7.46-7.39 (m, 2H), 6.87-6.83 (m, 2H), 6.83-6.77 (m, 2H), 6.51-6.45 (m, 2H), 6.20 (dd, J=9.2, 5.8 Hz, 1H), 4.67 (dt, J=9.9, 1.6 Hz, 1H), 4.62 (d, J=16.5 Hz, 1H), 4.44 (d, J=16.7 Hz, 1H), 4.37 (dd, J=10.0, 5.8 Hz, 1H), 3.77 (s, 3H), 3.75 (s, 3H). ¹³C NMR (126 MHz, CDCl₃) δ 168.5, 163.4, 163.3, 161.5, 161.4, 161.1 (t, J=14.7 Hz), 154.4, 152.5, 141.8, 132.5, 131.5, 128.0, 123.9, 122.8, 116.2, 114.8, 105.3, 98.7, 98.6, 98.5, 98.4, 67.4, 56.0, 55.8, 47.7, 46.5. HRMS-ESI+: m/z [M+H]⁺ calculated for C₂₄H₂₂O₄NF₂: 426.1511; found: 426.1511.

2-(1-(2,6-Difluoro-4-methoxyphenyl)-2-(4-methoxyphenoxy)ethyl)-6-(trifluoromethyl)isoindolin-1-one (EU1180-546) was synthesized using General Procedure G with a yield of 99%. ¹H NMR (500 MHz, CDCl₃) δ 8.11 (s, 1H), 7.79-7.73 (m, 1H), 7.55 (d, J=8.1 Hz, 1H), 6.87-6.77 (m, 4H), 6.51-6.45 (m, 2H), 6.19 (dd, J=9.5, 5.4 Hz, 1H), 4.73-4.65 (m, 2H), 4.53 (d, J=17.3 Hz, 1H), 4.35 (dd, J=10.1, 5.4 Hz, 1H), 3.77 (s, 3H), 3.74 (s, 3H). ¹³C NMR (126 MHz, CDCl₃) δ 167.2, 163.3 (d, J=11.6 Hz), 161.3 (d, J=13.5 Hz), 161.1, 153.4 (d, J=279.7 Hz), 145.1, 133.2, 130.9 (q, J=33.0 Hz), 128.3 (d, J=3.8 Hz), 124.0 (q, J=272.4 Hz), 123.6, 104. 9 (t, J=19.3 Hz), 99.2-97.5 (m), 67.4, 56.0, 55.8, 47.7, 46.8. HRMS-ESI+: m/z [M+H]⁺ calculated for C₂₄H₂₁O₄NF₅: 494.1385; found: 494.1388.

5-Chloro-2-(1-(2,6-difluoro-4-methoxyphenyl)-2-(4-methoxyphenoxy)ethyl)isoindolin-1-one (EU1180-547) was synthesized using General Procedure G with a yield of 89%. ¹H NMR (500 MHz, CDCl₃) δ 7.75 (d, J=8.1 Hz, 1H), 7.44-7.36 (m, 2H), 6.86-6.77 (m, 4H), 6.51-6.45 (m, 2H), 6.16 (dd, J=9.4, 5.6 Hz, 1H), 4.68-4.56 (m, 2H), 4.44 (d, J=17.0 Hz, 1H), 4.34 (dd, J=10.0, 5.6 Hz, 1H), 3.77 (s, 3H), 3.74 (s, 3H). ¹³C NMR (126 MHz, CDCl₃) δ 167.54, 163.34, 163.25, 161.37, 161.28, 161.15, 161.04, 154.46, 152.34, 143.33, 137.86, 130.96, 128.67, 125.11, 123.29, 116.12, 114.79, 105.21, 105.06, 104.91, 98.71, 98.48, 55.97, 55.80, 47.33, 46.63. HRMS-ESI+: m/z [M+H]⁺ calculated for C₂₄H₂₁O₄N³⁵ClF₂: 460.1122; found: 460.1124.

6-Chloro-2-(1-(2,6-difluoro-4-methoxyphenyl)-2-(4-methoxyphenoxy)ethyl)isoindolin-1-one (EU1180-548) was synthesized using General Procedure G with a yield of 62%. ¹H NMR (500 MHz, CDCl₃) δ 7.80 (d, J=1.9 Hz, 1H), 7.47 (dd, J=8.1, 2.0 Hz, 1H), 7.36-7.32 (m, 1H), 6.86-6.78 (m, 4H), 6.50-6.45 (m, 2H), 6.16 (dd, J=9.4, 5.5 Hz, 1H), 4.68-4.56 (m, 2H), 4.43 (d, J=16.9 Hz, 1H), 4.35 (dd, J=10.1, 5.6 Hz, 1H), 3.77 (s, 4H), 3.75 (s, 4H). ¹³C NMR (126 MHz, CDCl₃) δ 167.3, 163.4, 163.3, 161.4, 161.3, 161.2, 161.1, 154.5, 152.4, 139.9, 134.4, 134.2, 131.7, 124.1, 124.1, 124.1, 116.1, 114.8, 105.2, 105.0, 104.9, 98.7, 98.7, 98.5, 56.0, 55.8, 47.4, 46.7. HRMS-ESI+: m/z [M+H]⁺ calculated for C₂₄H₂₁O₄N³⁵ClF₂: 460.1122; found: 460.1123.

5-Bromo-2-(1-(2,6-difluoro-4-methoxyphenyl)-2-(4-methoxyphenoxy)ethyl)isoindolin-1-one (EU1180-549) was synthesized using General Procedure G with a yield of67% yield. ¹H NMR (500 MHz, CDCl₃) δ 7.71-7.68 (m, 1H), 7.59-7.55 (m, 2H), 6.85-6.78 (m, 4H), 6.51-6.46 (m, 2H), 6.15 (dd, J=9.4, 5.5 Hz, 1H), 4.68-4.57 (m, 2H), 4.44 (d, J=17.0 Hz, 1H), 4.34 (dd, J=10.1, 5.6 Hz, 1H), 3.77 (s, 3H), 3.75 (s, 3H). ¹³C NMR (126 MHz, CDCl₃) δ 167.6, 163.3, 163.2, 161.4, 161.3, 161.2, 154.5, 152.3, 143.6, 131.5, 131.4, 126.3, 126.2, 125.3, 116.1, 114.8, 105.0, 98.7, 98.5, 67.4, 56.0, 55.8, 47.3, 46.6. HRMS-ESI+: m/z [M+H]⁺ calculated for C₂₄H₂₁O₄N⁷⁹BrF₂: 504.0617; found: 504.0620.

6-Bromo-2-(1-(2,6-difluoro-4-methoxyphenyl)-2-(4-methoxyphenoxy)ethyl)isoindolin-1-one (EU1180-550) was synthesized using General Procedure G with a yield of 65%. ¹H NMR (500 MHz, CDCl₃) δ 7.97-7.93 (m, 1H), 7.64-7.59 (m, 1H), 7.29 (dt, J=8.0, 0.7 Hz, 1H), 6.85-6.77 (m, 4H), 6.50-6.45 (m, 2H), 6.16 (dd, J=9.4, 5.5 Hz, 1H), 4.68-4.61 (m, 1H), 4.58 (d, J=17.0 Hz, 1H), 4.41 (d, J=16.9 Hz, 1H), 4.34 (dd, J=10.0, 5.6 Hz, 1H), 3.79-3.75 (m, 3H), 3.75 (d, J=0.7 Hz, 3H). ¹³C NMR (126 MHz, CDCl₃) δ 167.1, 163.3, 163.2, 161.4, 161.3, 161.2, 161.1, 154.5, 152.3, 140.4, 134.5, 127.1, 127.1, 124.5, 122.1, 116.1, 114.8, 105.2, 105.0, 104.9, 98.7, 98.7, 98.5, 98.5, 56.0, 55.8, 47.5, 46.7. HRMS-ESI+: m/z [M+H]⁺ calculated for C₂₄H₂₁O₄N⁷⁹BrF₂: 504.0617; found: 504.0622.

2-(1-(2,6-Difluoro-4-methoxyphenyl)-2-(4-methoxyphenoxy)ethyl)-5-fluoroisoindolin-1-one (EU1180-551) was synthesized using General Procedure G with a yield of 69%. ¹H NMR (500 MHz, CDCl₃) δ 7.81 (dd, J=8.4, 5.0 Hz, 1H), 7.16-7.06 (m, 2H), 6.87-6.77 (m, 4H), 6.51-6.43 (m, 2H), 6.16 (dd, J=9.3, 5.6 Hz, 1H), 4.67-4.56 (m, 2H), 4.43 (d, J=16.9 Hz, 1H), 4.35 (dd, J=10.0, 5.7 Hz, 1H), 3.77 (s, 3H), 3.74 (s, 3H). ¹³C NMR (126 MHz, CDCl₃) δ 167.55, 166.19, 164.20, 163.36, 163.26, 161.39, 161.30, 161.14, 161.02, 154.46, 152.37, 144.15, 144.07, 128.48, 125.94, 125.87, 116.13, 115.96, 115.77, 114.79, 110.30, 110.12, 105.13 (t, J=19.3 Hz), 98.70, 98.47, 67.40, 55.96, 55.80, 47.41, 46.59. HRMS-ESI+: m/z [M+H]⁺ calculated for C₂₄H₂₁O₄NF₃: 444.1417; found: 444.1420.

2-(1-(2,6-Difluoro-4-methoxyphenyl)-2-(4-methoxyphenoxy)ethyl)-6-fluoroisoindolin-1-one (EU1180-552) was synthesized using General Procedure G with a yield of 42%. ¹H NMR (500 MHz, CDCl₃) δ 7.50 (p, J=7.6, 2.5, 0.6 Hz, 1H), 7.39-7.35 (m, 1H), 7.21 (ddd, J=9.0, 8.3, 2.5 Hz, 1H), 6.86-6.78 (m, 4H), 6.50-6.45 (m, 2H), 6.17 (dd, J=9.4, 5.6 Hz, 1H), 4.66 (tt, J=9.5, 1.6 Hz, 1H), 4.59 (d, J=16.3 Hz, 1H), 4.42 (d, J=16.6 Hz, 1H), 4.35 (dd, J=10.1, 5.6 Hz, 1H), 3.77 (s, 3H), 3.75 (s, 3H). ¹³C NMR (126 MHz, CDCl₃) δ 167.6, 163.9, 163.4, 163.3, 161.9, 161.4, 161.3, 161.2, 1545, 152.4, 137.1, 134.5 (d, J=8.9 Hz), 124.4, 124.3, 119.2, 119.0, 116.2, 114.8, 110.7, 110.5, 105.1, 98.7, 98.7, 98.5, 67.4, 56.0, 55.8, 47.3, 46.7. HRMS-ESI+: m/z [M+H]⁺ calculated for C₂₄H₂₁O₄NF₃: 444.1417; found: 444.1420.

### Example 55. Synthesis and Characterization of Compounds from EU1180-593 to EU1180-614

The synthetic route of the foregoing compounds included (1) synthesis of various acetophenone derivatives (Scheme 10), (2) conversion of the acetophenone derivatives to the respective benzylic amines via a reductive amination reaction (Scheme 11), and (3) performing a ring-closing step using the benzylic amines with various halogenated and non-halogenated methyl (2-bromomethyl)benzoate derivatives to furnish the desired isoindolinone ring (Scheme 12).

Scheme 10 illustrates the synthesis of various substituted acetophenone derivatives. A Williamson etherification step with 6-methylpyridinol produced the substituted acetophenone derivatives. See Lindsay, et al., Organic & Biomolecular Chemistry, 2019, 17, 7408-7415.

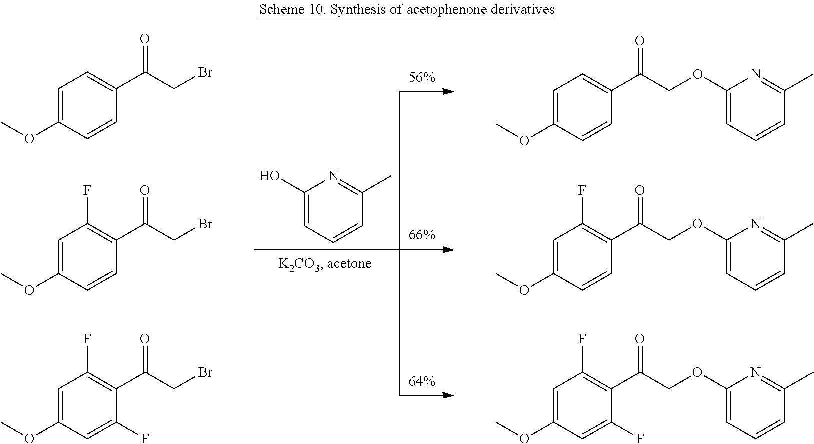

Scheme 11 illustrates the reductive amination step of the acetophenone derivatives. Reductive amination was carried out in one pot with ammonium acetate and sodium cyanoborohydride in methanol to afford the advanced benzylic secondary amine intermediates.

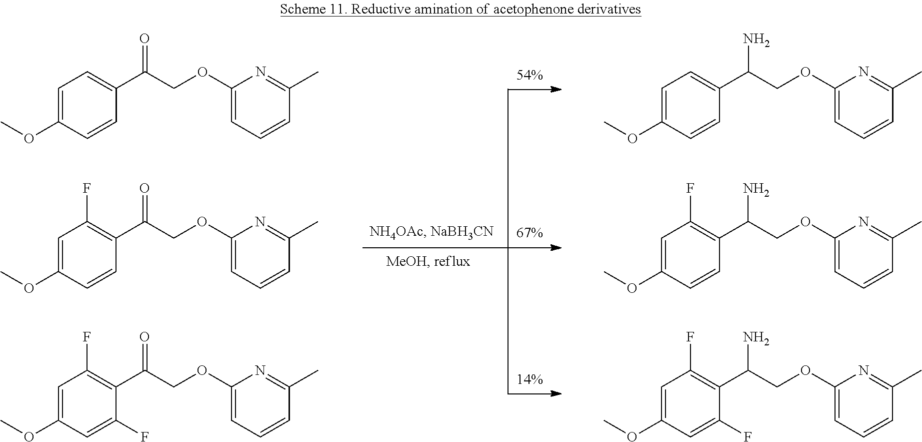

Scheme 12 illustrates the ring closing step performed using the benzylic amine derivatives from Scheme 11 and 2-bromometh 1 meth lbenzoates. See General Procedure G for more details.

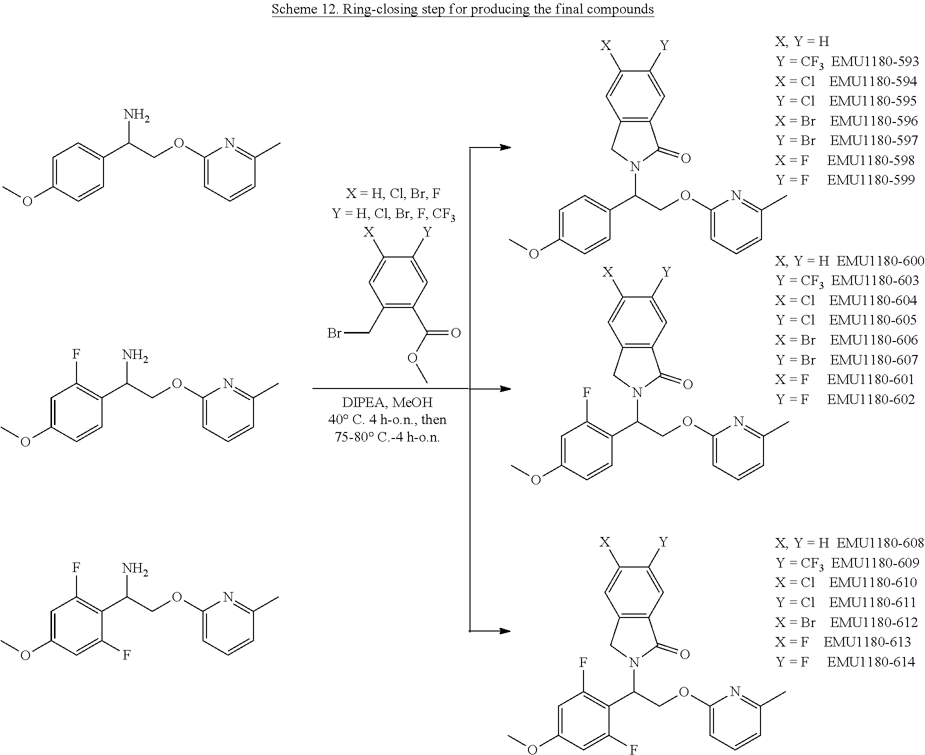

2-(1-(4-Methoxyphenyl)-2-((6-methylpyridin-2-yl)oxy)ethyl)-6-(trifluoromethyl)isoindolin-1-one (EW1180-593) was synthesized using General Procedure G with a yield of 65%. ¹H NMR (600 MHz, CDCl₃) δ 8.11 (s, 1H), 7.74 (d, J=7.7 Hz, 1H), 7.48 (s, 1H), 7.41-7.34 (m, 3H), 6.92-6.87 (m, 2H), 6.67 (d, J=7.2 Hz, 1H), 6.50 (d, J=8.2 Hz, 1H), 5.94 (dd, J=8.3, 4.9 Hz, 1H), 5.02 (dd, J=11.5, 8.3 Hz, 1H), 4.86 (dd, J=11.5, 4.9 Hz, 1H), 4.64 (d, J=17.4 Hz, 1H), 4.27 (d, J=17.4 Hz, 1H), 3.79 (s, 3H), 2.39 (s, 3H). ¹³C NMR (151 MHz, CDCl₃) δ 167.4, 162.6, 159.5, 156.1, 145.1, 139.1, 133.6, 130.9 (q, J=32.7 Hz), 129.2, 129.1, 128.1 (q, J=3.5 Hz), 126.8-121.3 (m), 123.5, 121.2 (q, J=3.9 Hz), 116.3, 114.4, 107.8, 64.6, 55.4, 53.6, 47.4, 24.1. HRMS-ESI+: m/z [M+H]⁺ calculated for C₂₄H₂₂F₃N₂O₃: 443.1582; found: 443.1573.

5-Chloro-2-(1-(4-methoxyphenyl)-2-((6-methylpyridin-2-yl)oxy)ethyl)isoindolin-1-one (EU1180-594) was synthesized using General Procedure G with a yield of 60%. ¹H NMR (600 MHz, CDCl₃) δ 7.78 (d, J=8.1 Hz, 1H), 7.42 (dd, J=8.3, 7.0 Hz, 2H), 7.40-7.35 (m, 3H), 6.93-6.88 (m, 2H), 6.70 (d, J=7.2 Hz, 1H), 6.52 (d, J=8.2 Hz, 1H), 5.93 (dd, J=8.2, 5.0 Hz, 1H), 5.01 (dd, J=11.4, 8.2 Hz, 1H), 4.86 (dd, J=11.4, 5.0 Hz, 1H), 4.56 (d, J=17.1 Hz, 1H), 4.21 (d, J=17.1 Hz, 1H), 3.81 (s, 3H), 2.42 (s, 3H). ¹³C NMR (151 MHz, CDCl₃) δ 167.8, 162.6, 159.4, 156.1, 143.3, 139.1, 137.7, 131.3, 129.3, 129.1, 128.6, 125.1, 123.2, 116.3, 114.3, 107.8, 64.7, 55.4, 53.5, 47.0, 24.1. HRMS-ESI+: m/z [M+H]⁺ calculated for C₂H₂₂O₃N₂³⁵Cl: 409.1314; found: 409.1307.

6-Chloro-2-(1-(4-methoxyphenyl)-2-((6-methylpyridin-2-yl)oxy)ethyl)isoindolin-1-one (EU1180-595) was synthesized using General Procedure G with a yield of 83%. ¹H NMR (600 MHz, CDCl₃) δ 7.80 (d, J=1.9 Hz, 1H), 7.43 (dd, J=8.0, 2.0 Hz, 1H), 7.39 (dd, J=8.2, 7.2 Hz, 1H), 7.37-7.33 (m, 2H), 7.28 (d, J=8.0 Hz, 1H), 6.91-6.86 (m, 2H), 6.67 (d, J=7.2 Hz, 1H), 6.49 (d, J=8.2 Hz, 1H), 5.91 (dd, J=8.2, 5.0 Hz, 1H), 4.98 (dd, J=11.4, 8.3 Hz, 1H), 4.84 (dd, J=11.4, 5.0 Hz, 1H), 4.53 (d, J=17.0 Hz, 1H), 4.18 (d, J=17.0 Hz, 1H), 3.78 (s, 3H), 2.39 (s, 3H). ¹³C NMR (151 MHz, CDCl₃) δ 167.5, 162.6, 159.4, 156.1, 139.8, 139.0, 134.6, 134.3, 131.5, 129.3, 129.1, 124.0, 124.0, 116.3, 114.3, 107.8, 64.6, 55.4, 53.5, 47.0, 24.2. HRMS-ESI+: m/z [M+H]⁺ calculated for C₂H₂₂O₃N₂³⁵Cl: 409.1314; found: 409.1310.

5-Bromo-2-(1-(4-methoxyphenyl)-2-((6-methylpyridin-2-yl)oxy)ethyl)isoindolin-1-one (EU1180-596) was synthesized using General Procedure G with a yield of 69%. ¹H NMR (600 MHz, CDCl₃) δ 7.69 (d, J=8.1 Hz, 1H), 7.55 (dd, J=8.1, 1.6 Hz, 1H), 7.50 (s, 1H), 7.40 (dd, J=8.2, 7.3 Hz, 1H), 7.37-7.33 (m, 2H), 6.91-6.85 (m, 2H), 6.68 (d, J=7.2 Hz, 1H), 6.49 (d, J=8.2 Hz, 1H), 5.90 (dd, J=8.2, 5.0 Hz, 1H), 4.99 (dd, J=11.4, 8.2 Hz, 1H), 4.84 (dd, J=11.4, 5.0 Hz, 1H), 4.54 (d, J=17.1 Hz, 1H), 4.19 (d, J=17.1 Hz, 1H), 3.78 (s, 3H), 2.40 (s, 3H). ¹³C NMR (151 MHz, CDCl₃) δ 167.8, 162.6, 159.4, 156.1, 143.5, 139.1, 131.8, 131.4, 129.3, 129.0, 126.2, 126.0, 125.3, 116.3, 114.3, 107.8, 64.6, 55.4, 53.5, 46.9, 24.1. HRMS-ESI+: m/z [M+H]⁺ calculated for C₂₃H₂₂O₃N₂⁷⁹Br: 453.0808; found: 453.0806.

6-Bromo-2-(1-(4-methoxyphenyl)-2-((6-methylpyridin-2-yl)oxy)ethyl)isoindolin-1-one (EU1180-597) was synthesized using General Procedure G with a yield of 88%. ¹H NMR (600 MHz, CDCl₃) δ 7.97-7.95 (m, 1H), 7.59 (dd, J=8.0, 1.8 Hz, 1H), 7.40 (t, J=7.7 Hz, 1H), 7.38-7.31 (m, 2H), 7.23 (d, J=8.0 Hz, 1H), 6.92-6.85 (m, 2H), 6.68 (d, J=7.2 Hz, 1H), 6.50 (d, J=8.2 Hz, 1H), 5.91 (dd, J=8.2, 5.0 Hz, 1H), 4.98 (dd, J=11.4, 8.2 Hz, 1H), 4.84 (dd, J=11.4, 5.0 Hz, 1H), 4.52 (d, J=17.1 Hz, 1H), 4.16 (d, J=17.1 Hz, 1H), 3.79 (s, 3H), 2.40 (s, 3H). ¹³C NMR (151 MHz, CDCl₃) δ 167.3, 162.6, 159.4, 156.1, 140.3, 139.1, 134.9, 134.3, 129.3, 129.1, 127.1, 124.4, 122.0, 116.3, 114.3, 107.8, 64.6, 55.4, 53.5, 47.1, 24.1. HRMS-ESI+: m/z [M+H]⁺ calculated for C₂₃H₂₂O₃N₂⁷⁹Br: 453.0808; found: 453.0805.

5-Fluoro-2-(1-(4-methoxyphenyl)-2-((6-methylpyridin-2-yl)oxy)ethyl)isoindolin-1-one (EU1180-598) was synthesized using General Procedure G with a yield of 47%. ¹H NMR (600 MHz, CDCl₃) δ 7.81 (dd, J=8.4, 5.0 Hz, 1H), 7.40 (dd, J=8.2, 7.2 Hz, 1H), 7.38-7.33 (m, 2H), 7.12 (td, J=8.8, 2.2 Hz, 1H), 7.04 (dd, J=8.2, 2.2 Hz, 1H), 6.92-6.86 (m, 2H), 6.68 (d, J=7.2 Hz, 1H), 6.50 (d, J=8.2 Hz, 1H), 5.90 (dd, J=8.1, 5.0 Hz, 1H), 4.99 (dd, J=11.4, 8.1 Hz, 1H), 4.84 (dd, J=11.4, 5.0 Hz, 1H), 4.55 (d, J=17.0 Hz, 1H), 4.19 (d, J=17.0 Hz, 1H), 3.79 (s, 3H), 2.40 (s, 3H). ¹³C NMR (151 MHz, CDCl₃) δ 167.8, 165.1 (d, J=250.7 Hz), 162.6, 159.4, 156.1, 144.1 (d, J=10.0 Hz), 139.1, 129.4, 129.1, 128.9 (d, J=2.5 Hz), 125.9 (d, J=9.9 Hz), 116.3, 115.8 (d, J=23.5 Hz), 114.3, 110.2 (d, J=24.0 Hz), 107.8, 64.7, 55.4, 53.5, 47.1 (d, J=2.7 Hz), 24.1. HRMS-ESI+: m/z [M+H]⁺ calculated for C₂₃H₂₂O₃N₂F: 393.1609; found: 393.1605.

6-Fluoro-2-(1-(4-methoxyphenyl)-2-((6-methylpyridin-2-yl)oxy)ethyl)isoindolin-1-one (EU1180-599) was synthesized using General Procedure G with a yield of 74%. ¹H NMR (600 MHz, CDCl₃) δ 7.50 (dd, J=7.7, 2.5 Hz, 1H), 7.39 (t, J=7.7 Hz, 1H), 7.38-7.34 (m, 2H), 7.31 (dd, J=8.3, 4.4 Hz, 1H), 7.18 (td, J=8.6, 2.5 Hz, 1H), 6.91-6.87 (m, 2H), 6.67 (d, J=7.2 Hz, 1H), 6.49 (d, J=8.2 Hz, 1H), 5.91 (dd, J=8.2, 5.0 Hz, 1H), 4.99 (dd, J=11.4, 8.3 Hz, 1H), 4.84 (dd, J=11.4, 5.1 Hz, 1H), 4.53 (d, J=16.7 Hz, 1H), 4.18 (d, J=16.7 Hz, 1H), 3.78 (s, 3H), 2.39 (s, 3H). ¹³C NMR (151 MHz, CDCl₃) δ 167.8 (d, J=3.4 Hz), 162.8 (d, J=246.7 Hz), 162.6, 159.4, 156.1, 139.0, 137.0, 137.0, 134.8 (d, J=8.4 Hz), 129.4, 129.1, 124.2 (d, J=8.3 Hz), 118.9 (d, J=23.8 Hz), 116.3, 114.3, 110.6 (d, J=23.3 Hz), 107.8, 64.6, 55.4, 53.6, 46.9, 24.2. HRMS-ESI+: m/z [M+H]⁺ calculated for C₂₃H₂₂O₃N₂F: 393.1609; found: 393.1603.

2-(1-(2-Fluoro-4-methoxyphenyl)-2-((6-methylpyridin-2-yl)oxy)ethyl)isoindolin-1-one (EU1180-600) was synthesized using General Procedure G with a yield of 20%. ¹H NMR (600 MHz, CDCl₃) δ 7.84-7.81 (m, 1H), 7.54-7.35 (m, 4H), 6.76-6.58 (m, 3H), 6.51-6.45 (m, 1H), 6.06-5.96 (m, 1H), 5.07-5.02 (m, 1H), 4.86-4.79 (m, 1H), 4.63 (d, J=16.8 Hz, 1H), 4.32 (d, J=16.7 Hz, 1H), 3.86-3.73 (m, 3H), 2.45-2.32 (m, 3H). ¹³C NMR (151 MHz, CDCl₃) δ 168.6, 162.7, 162.6, 161.0, 160.9, 160.9, 156.2, 141.8, 139.0, 132.9, 131.4, 130.7, 130.7, 128.0, 123.9, 122.8, 116.7, 116.6, 116.3, 116.2, 110.2, 110.2, 107.8, 102.3, 102.1, 64.3, 64.3, 55.7, 50.2, 48.2, 48.2, 24.2. HRMS (ESI): m/z calcd. for C₂₃H₂₂O₃N₂F: 393.1609 [M+H]⁺; found: 393.1615.

5-Fluoro-2-(1-(2-fluoro-4-methoxyphenyl)-2-((6-methylpyridin-2-yl)oxy)ethyl)isoindolin-1-one (EU1180-601) was synthesized using General Procedure G with a yield of 41%. ¹H NMR (500 MHz, CDCl₃) δ 7.79 (dd, J=8.4, 5.0 Hz, 1H), 7.44 (t, J=8.6 Hz, 1H), 7.40 (dd, J=8.2, 7.2 Hz, 1H), 7.14-7.08 (m, 1H), 7.06 (dd, J=8.2, 2.2 Hz, 1H), 6.72-6.66 (m, 2H), 6.63 (dd, J=12.3, 2.5 Hz, 1H), 6.51-6.46 (m, 1H), 5.98 (dd, J=8.4, 5.1 Hz, 1H), 5.05 (ddd, J=11.3, 8.4, 1.3 Hz, 1H), 4.80 (dd, J=11.4, 5.2 Hz, 1H), 4.62 (d, J=17.1 Hz, 1H), 4.29 (d, J=17.0 Hz, 1H), 3.78 (s, 3H), 2.38 (s, 3H). ¹³C NMR (126 MHz, CDCl₃) δ 167.6, 166.1, 164.1, 162.7, 162.6, 161.0, 160.9, 160.8, 156.2, 144.1, 144.1, 139.0, 130.7, 130.6, 128.9, 125.9, 125.8, 115.9, 115.7, 110.2, 110.2, 110.0, 108.0, 107.7, 102.3, 102.1, 64.3, 55.7, 50.3, 47.9, 24.2. HRMS (ESI): m/z calcd. for C₂₃H₂₁O₃N₂F₂: 411.1515 [M+H]⁺; found: 411.1520.

6-Fluoro-2-(1-(2-fluoro-4-methoxyphenyl)-2-((6-methylpyridin-2-yl)oxy)ethyl)isoindolin-1-one (EU1180-602) was synthesized using General Procedure G with a yield of 49%. ¹H NMR (600 MHz, CDCl₃) δ 7.48 (dd, J=7.8, 2.5 Hz, 1H), 7.45 (t, J=8.5 Hz, 1H), 7.42-7.37 (m, 1H), 7.33 (dd, J=8.3, 4.4 Hz, 1H), 7.23-7.16 (m, 1H), 6.73-6.60 (m, 3H), 6.49 (d, J=8.2 Hz, 1H), 5.99 (dd, J=8.5, 5.1 Hz, 1H), 5.05 (dd, J=11.4, 8.5 Hz, 1H), 4.81 (dd, J=11.4, 5.1 Hz, 1H), 4.61 (d, J=16.7 Hz, 1H), 4.28 (d, J=16.7 Hz, 1H), 3.79 (s, 3H), 2.38 (s, 3H). ¹³C NMR (151 MHz, CDCl₃) δ 167.6, 167.5, 163.7, 162.6, 162.6, 162.1, 161.0, 160.9, 156.2, 139.1, 137.1, 137.1, 134.9, 134.8, 130.7, 130.6, 124.3, 124.2, 119.0, 118.8, 116.4, 116.3, 116.3, 116.3, 110.7, 110.5, 110.2, 110.2, 107.8, 102.3, 102.2, 64.2, 64.2, 55.7, 50.4, 47.8, 47.8, 24.2. HRMS (ESI): m/z calcd. for C₂₃H₂₁O₃N₂F₂: 411.1515 [M+H]⁺; found: 411.1521.

2-(1-(2-Fluoro-4-methoxyphenyl)-2-((6-methylpyridin-2-yl)oxy)ethyl)-6-(trifluoromethyl)isoindolin-1-one (EU1180-603) was synthesized using General Procedure G with a yield of 51%. ¹H NMR (600 MHz, CDCl₃) δ 8.09 (s, 1H), 7.76-7.73 (m, 1H), 7.51 (d, J=7.9 Hz, 1H), 7.46 (t, J=8.6 Hz, 1H), 7.40 (t, J=7.7 Hz, 1H), 6.71 (dd, J=8.6, 2.6 Hz, 1H), 6.67 (d, J=7.2 Hz, 1H), 6.64 (dd, J=12.3, 2.6 Hz, 1H), 6.49 (d, J=8.2 Hz, 1H), 6.01 (dd, J=8.5, 5.0 Hz, 1H), 5.08 (ddd, J=11.5, 8.5, 1.2 Hz, 1H), 4.82 (dd, J=11.4, 5.0 Hz, 1H), 4.71 (d, J=17.4 Hz, 1H), 4.38 (d, J=17.4 Hz, 1H), 3.79 (s, 3H), 2.38 (s, 3H). ¹³C NMR (151 MHz, CDCl₃) δ 167.2, 162.6, 162.6, 161.1, 161.0, 161.0, 156.2, 145.1, 139.1, 133.6, 131.2, 131.0, 130.8, 130.7, 130.6, 130.6, 128.2, 128.2, 128.1, 128.1, 126.7, 124.9, 123.5, 123.1, 121.3, 121.3, 121.2, 121.2, 116.4, 116.2, 116.1, 110.3, 110.3, 107.7, 102.4, 102.2, 64.2, 64.2, 55.7, 50.4, 48.2, 48.2, 24.2. HRMS (ESI): m/z calcd. for C₂₄H₂₁O₃N₂F₄: 461.1483 [M+H]⁺; found: 461.1490.

5-Chloro-2-(1-(2-fluoro-4-methoxyphenyl)-2-((6-methylpyridin-2-yl)oxy)ethyl)isoindolin-1-one (EU1180-604) was synthesized using General Procedure G with a yield of 48%. ¹H NMR (600 MHz, CDCl₃) δ 7.74 (d, J=8.1 Hz, 1H), 7.46-7.35 (m, 4H), 6.71-6.66 (m, 2H), 6.64 (dd, J=12.3, 2.6 Hz, 1H), 6.49 (d, J=8.2 Hz, 1H), 5.97 (dd, J=8.4, 5.1 Hz, 1H), 5.05 (ddd, J=11.5, 8.4, 1.2 Hz, 1H), 4.81 (dd, J=11.4, 5.1 Hz, 1H), 4.62 (d, J=17.0 Hz, 1H), 4.29 (d, J=17.1 Hz, 1H), 3.79 (s, 3H), 2.39 (s, 3H). ¹³C NMR (151 MHz, CDCl₃) δ 167.6, 162.6, 162.6, 161.0, 161.0, 156.3, 143.3, 139.1, 137.7, 131.4, 130.7, 130.6, 128.6, 125.1, 123.2, 116.4, 116.3, 116.3, 110.2, 110.2, 107.8, 102.3, 102.2, 64.2, 64.2, 55.8, 50.4, 47.8, 47.8, 29.9, 24.2. HRMS (ESI): m/z calcd. for CH₂₁H₂₁O₃N₂³⁵ClF: 427.1219 [M+H]⁺; found: 427.1229.

6-Chloro-2-(1-(2-fluoro-4-methoxyphenyl)-2-((6-methylpyridin-2-yl)oxy)ethyl)isoindolin-1-one (EU1180-605) was synthesized using General Procedure G with a yield of 15%. ¹H NMR (600 MHz, CDCl₃) δ 7.78 (d, J=2.0 Hz, 1H), 7.47-7.38 (m, 3H), 7.31 (d, J=8.0 Hz, 1H), 6.72-6.66 (m, 2H), 6.63 (dd, J=12.3, 2.5 Hz, 1H), 6.49 (d, J=8.2 Hz, 1H), 5.98 (dd, J=8.4, 5.1 Hz, 1H), 5.05 (ddd, J=11.5, 8.4, 1.2 Hz, 1H), 4.81 (dd, J=11.4, 5.1 Hz, 1H), 4.62 (d, J=17.0 Hz, 1H), 4.29 (d, J=16.9 Hz, 1H), 3.79 (s, 3H), 2.39 (s, 3H). ¹³C NMR (151 MHz, CDCl₃) δ 167.3, 162.6, 161.1, 161.0, 161.0, 156.2, 140.0, 139.1, 134.6, 134.3, 131.6, 130.7, 130.6, 124.1, 124.0, 116.4, 116.3, 110.2, 110.2, 107.8, 102.3, 102.2, 64.3, 55.8, 50.4, 47.9, 48.0, 24.1. HRMS (ESI): m/z calcd. for CH₂₃H₂₁O₃N₂³⁵ClF: 427.1219 [M+H]⁺; found: 427.1227.

5-Bromo-2-(1-(2-fluoro-4-methoxyphenyl)-2-((6-methylpyridin-2-yl)oxy)ethyl)isoindolin-1-on (EU1180-606) was synthesized using General Procedure G with a yield of 52%. ¹H NMR (500 MHz, CDCl₃) δ 7.67 (d, J=8.0 Hz, 1H), 7.57-7.51 (m, 2H), 7.44 (t, J=8.6 Hz, 1H), 7.40 (t, J=7.7 Hz, 1H), 6.72-6.65 (m, 2H), 6.63 (dd, J=12.3, 2.6 Hz, 1H), 6.48 (d, J=8.2 Hz, 1H), 5.97 (dd, J=8.4, 5.1 Hz, 1H), 5.04 (dd, J=11.4, 8.5 Hz, 1H), 4.80 (dd, J=11.4, 5.1 Hz, 1H), 4.61 (d, J=17.1 Hz, 1H), 4.29 (d, J=17.1 Hz, 1H), 3.78 (s, 3H), 2.38 (s, 3H). ¹³C NMR (126 MHz, CDCl₃) δ 167.6, 162.7, 162.6, 160.9, 160.8, 156.2, 143.6, 139.1, 131.8, 131.4, 130.6, 130.6, 126.2, 126.0, 125.3, 116.3, 110.2, 110.2, 107.7, 102.3, 102.1, 64.2, 55.7, 50.3, 47.7, 24.2. HRMS (ESI): m/z calcd. for C₂₃H₂₁O₃N₂⁷⁹BrF: 471.0714 [M+H]⁺; found: 471.0723.

6-Bromo-2-(1-(2-fluoro-4-methoxyphenyl)-2-((6-methylpyridin-2-yl)oxy)ethyl)isoindolin-1-one (EU1180-607) was synthesized using General Procedure G with a yield of 55%. ¹H NMR (500 MHz, CDCl₃) δ 7.93 (d, J=1.8 Hz, 1H), 7.59 (dt, J=8.1, 1.5 Hz, 1H), 7.45-7.37 (m, 2H), 7.25 (d, J=8.1 Hz, 1H), 6.71-6.66 (m, 2H), 6.63 (dd, J=12.3, 2.4 Hz, 1H), 6.48 (d, J=8.2 Hz, 1H), 5.98 (dd, J=8.5, 5.1 Hz, 1H), 5.04 (ddd, J=11.3, 8.5, 1.3 Hz, 1H), 4.80 (ddd, J=11.4, 5.1, 1.1 Hz, 1H), 4.59 (d, J=17.1 Hz, 1H), 4.26 (d, J=17.1 Hz, 1H), 3.78 (s, 3H), 2.38 (s, 3H). ¹³C NMR (126 MHz, CDCl₃) δ 166.9, 165.6, 162.6, 160.8, 140.4, 139.0, 134.8, 134.3, 130.6, 130.6, 127.1, 124.4, 122.0, 116.3, 110.2, 107.7, 102.3, 102.1, 64.2, 55.7, 50.4, 47.9, 24.2. HRMS (ESI): m/z calcd. for C₂₃H₂₁O₃N₂⁷⁹BrF: 471.0714 [M+H]⁺; found: 471.0722.

2-(1-(2,6-Difluoro-4-methoxyphenyl)-2-((6-methylpyridin-2-yl)oxy)ethyl)isoindolin-1-one (EU1180-608) was synthesized using General Procedure G with a yield of 29%. ¹H NMR (600 MHz, CDCl₃) δ 7.80 (d, J=7.6 Hz, 1H), 7.52-7.47 (m, 1H), 7.43-7.37 (m, 3H), 6.66 (d, J=7.2 Hz, 1H), 6.48 (dd, J=9.1, 4.7 Hz, 3H), 6.28 (dd, J=9.7, 5.1 Hz, 1H), 5.15-5.09 (m, 1H), 4.76-4.69 (m, 2H), 4.44 (d, J=16.7 Hz, 1H), 3.77 (s, 3H), 2.37 (s, 3H). ¹³C NMR (151 MHz, CDCl₃) δ 168.5, 163.3, 163.2, 162.6, 161.6, 161.6, 161.1, 161.0, 160.9, 156.1, 141.9, 139.0, 132.5, 131.4, 127.9, 123.9, 122.8, 116.2, 107.9, 105.9, 105.7, 105.6, 98.6, 98.6, 98.5, 98.4, 63.8, 63.8, 63.8, 56.0, 47.5, 47.5, 47.5, 46.3, 24.1. HRMS (ESI): m/z calcd. for C₂₃H₂₁O₃N₂F₂: 411.1515 [M+H]⁺; found: 411.1507.

2-(1-(2,6-Difluoro-4-methoxyphenyl)-2-((6-methylpyridin-2-yl)oxy)ethyl)-6-(trifluoromethyl)isoindolin-1-one (EU1180-609) was synthesized using General Procedure G with a yield of 49%. ¹H NMR (600 MHz, CDCl₃) δ 8.07-8.04 (m, 1H), 7.77-7.73 (m, 1H), 7.56-7.52 (m, 1H), 7.39 (t, J=7.7 Hz, 1H), 6.68-6.63 (m, 1H), 6.50-6.45 (m, 3H), 6.27 (dd, J=10.0, 4.7 Hz, 1H), 5.17-5.11 (m, 1H), 4.82 (d, J=17.3 Hz, 1H), 4.72 (dd, J=11.6, 4.8 Hz, 1H), 4.52 (d, J=17.3 Hz, 1H), 3.78 (s, 3H), 2.35 (s, 3H). ¹³C NMR (151 MHz, CDCl₃) δ 167.2, 167.2, 163.2, 163.1, 162.6, 162.5, 161.6, 161.5, 161.3, 161.2, 161.1, 156.1, 156.0, 145.4, 145.2, 139.1, 139.1, 133.5, 133.3, 131.2, 131.0, 130.8, 130.8, 130.7, 130.5, 128.2, 128.2, 128.2, 128.2, 128.1, 128.1, 128.1, 128.0, 126.7, 124.9, 124.9, 123.5, 123.4, 123.1, 123.1, 121.3, 121.3, 121.3, 121.2, 121.20, 121.2, 121.2, 116.4, 116.2, 107.9, 107.9, 105.4, 105.3, 105.1, 98.7, 98.7, 98.5, 98.5, 63.9, 63.9, 63.7, 63.6, 63.6, 56.6, 56.0, 48.0, 47.9, 47.6, 47.6, 47.6, 47.1, 46.6, 24.1. HRMS (ESI): m/z calcd. for C₂₄H₂₀O₃N₂F₅: 479.1389 [M+H]⁺; found: 479.1382.

5-Chloro-2-(1-(2,6-difluoro-4-methoxyphenyl)-2-((6-methylpyridin-2-yl)oxy)ethyl)isoindolin-1-one (EU1180-610) was synthesized using General Procedure G with a yield of 33%. ¹H NMR (600 MHz, CDCl₃) δ 7.71 (d, J=8.0 Hz, 1H), 7.41-7.37 (m, 3H), 6.67 (d, J=7.2 Hz, 1H), 6.48 (d, J=9.5 Hz, 3H), 6.24 (dd, J=9.9, 4.9 Hz, 1H), 5.14-5.07 (m, 1H), 4.74-4.67 (m, 2H), 4.43 (d, J=16.9 Hz, 1H), 3.78 (s, 3H), 2.37 (s, 3H). ¹³C NMR (151 MHz, CDCl₃) δ 167.5, 163.2, 163.1, 162.5, 161.6, 161.5, 161.2, 161.1, 161.0, 156.1, 143.4, 139.1, 137.7, 131.0, 128.6, 125.1, 123.2, 116.3, 107.9, 105.6, 105.5, 105.3, 98.7, 98.6, 98.5, 98.5, 63.7, 63.7, 63.6, 56.0, 47.2, 47.2, 47.2, 46.5, 24.1. HRMS (ESI): m/z calcd. for C₂₃H₂₀O₃N₂³⁵ClF₂: 445.1125 [M+H]⁺; found: 445.1120.

6-Chloro-2-(1-(2,6-difluoro-4-methoxyphenyl)-2-((6-methylpyridin-2-yl)oxy)ethyl)isoindolin-1-one (EU1180-611) was synthesized using General Procedure G with a yield of 28%. ¹H NMR (600 MHz, CDCl₃) δ 7.75 (d, J=1.9 Hz, 1H), 7.46 (dt, J=8.1, 1.5 Hz, 1H), 7.39 (dd, J=8.3, 7.2 Hz, 1H), 7.34 (d, J=8.1 Hz, 1H), 6.67 (d, J=7.2 Hz, 1H), 6.50-6.44 (m, 3H), 6.24 (dd, J=9.9, 4.9 Hz, 1H), 5.15-5.08 (m, 1H), 4.74-4.68 (m, 2H), 4.42 (d, J=16.9 Hz, 1H), 3.78 (s, 3H), 2.36 (s, 3H). ¹³C NMR (151 MHz, CDCl₃) δ 167.3, 163.2, 163.1, 162.5, 161.6, 161.5, 161.2, 161.1, 161.0, 156.1, 140.0, 139.1, 134.3, 134.2, 131.6, 124.1, 116.3, 107.9, 105.6, 105.4, 105.3, 98.7, 98.6, 98.5, 98.5, 63.7, 56.0, 47.3, 47.3, 47.3, 46.6, 24.1. HRMS (ESI): m/z calcd. for C₂₃H₂₀O₃N₂³⁵ClF₂: 445.1125 [M+H]⁺; found: 445.1117.

5-Bromo-2-(1-(2,6-difluoro-4-methoxyphenyl)-2-((6-methylpyridin-2-yl)oxy)ethyl)isoindolin-1-one (EU1180-612) was synthesized using General Procedure G with a yield of 24%. ¹H NMR (600 MHz, CDCl₃) δ 7.65 (d, J=8.1 Hz, 1H), 7.58-7.50 (m, 2H), 7.39 (t, J=7.7 Hz, 1H), 6.67 (d, J=7.2 Hz, 1H), 6.48 (d, J=9.5 Hz, 3H), 6.23 (dd, J=9.9, 4.9 Hz, 1H), 5.12-5.07 (m, 1H), 4.74-4.68 (m, 2H), 4.43 (d, J=17.0 Hz, 1H), 3.78 (s, 3H), 2.37 (s, 3H) ¹³C NMR (151 MHz, CDCl₃) δ 167.6, 163.2, 163.1, 162.5, 161.6, 161.5, 161.2, 161.1, 161.0, 156.1, 143.7, 139.1, 131.5, 131.4, 126.2, 126.1, 125.3, 116.3, 107.9, 105.6, 105.4, 105.3, 98.7, 98.6, 98.5, 98.5, 66.0, 63.7, 63.7, 63.6, 56.0, 47.2, 47.1, 47.1, 46.5, 24.1, 15.4. HRMS (ESI): m/z calcd. for C₂₃H₂₀O₃N₂⁷⁹BrF₂: 489.0620 [M+H]⁺; found: 489.0614.

2-(1-(2,6-Difluoro-4-methoxyphenyl)-2-((6-methylpyridin-2-yl)oxy)ethyl)-5-fluoroisoindolin-1-one (EU1180-613) was synthesized using General Procedure G with a yield of 27%. ¹H NMR (600 MHz, CDCl₃) δ 7.78-7.74 (m, 1H), 7.39 (t, J=7.7 Hz, 1H), 7.13-7.07 (m, 2H), 6.66 (d, J=7.2 Hz, 1H), 6.50-6.45 (m, 3H), 6.24 (dd, J=9.8, 4.9 Hz, 1H), 5.14-5.08 (m, 1H), 4.75-4.68 (m, 2H), 4.42 (d, J=16.9 Hz, 1H), 3.77 (s, 3H), 2.36 (s, 3H). ¹³C NMR (151 MHz, CDCl₃) δ 167.6, 166.0, 164.3, 163.2, 163.1, 162.5, 161.6, 161.5, 161.2, 161.1, 161.0, 156.1, 144.2, 144.2, 139.0, 128.6, 128.5, 125.9, 125.9, 116.3, 115.8, 115.7, 110.2, 110.0, 107.9, 105.7, 105.5, 105.4, 98.7, 98.6, 98.5, 98.5, 66.0, 63.7, 63.7, 63.7, 56.0, 47.3, 47.3, 47.3, 47.3, 46.5, 24.1, 15.4. HRMS (ESI): m/z calcd. for C₂₃H₂₀O₃N₂F₃: 429.1421 [M+H]⁺; found: 429.1415.

2-(1-(2,6-Difluoro-4-methoxyphenyl)-2-((6-methylpyridin-2-yl)oxy)ethyl)-6-fluoroisoindolin-1-one (EU1180-614) was synthesized using General Procedure G with a yield of 25%. ¹H NMR (600 MHz, CDCl₃) δ 7.45 (dd, J=7.7, 2.4 Hz, 1H), 7.39 (dd, J=8.2, 7.2 Hz, 1H), 7.36 (dd, J=8.3, 4.4 Hz, 1H), 7.20 (td, J=8.7, 2.5 Hz, 1H), 6.66 (d, J=7.2 Hz, 1H), 6.50-6.45 (m, 3H), 6.25 (dd, J=9.8, 4.9 Hz, 1H), 5.15-5.09 (m, 1H), 4.74-4.68 (m, 2H), 4.41 (d, J=16.6 Hz, 1H), 3.78 (s, 3H), 2.36 (s, 3H). ¹³C NMR (151 MHz, CDCl₃) δ 167.6, 167.5, 163.7, 163.2, 163.2, 162.5, 162.0, 161.6, 161.5, 161.2, 161.1, 161.0, 156.1, 139.1, 137.2, 137.2, 134.6, 134.5, 124.3, 124.2, 119.1, 118.9, 116.3, 110.7, 110.5, 107.9, 105.6, 105.5, 105.3, 98.7, 98.6, 98.5, 98.5, 63.7, 63.7, 63.7, 56.0, 47.2, 47.2, 47.2, 46.6, 24.1. HRMS (ESI): m/z calcd. for C₂₃H₂₀O₃N₂F₃: 429.1421 [M+H]⁺; found: 429.1414.

### Example 56. Biological Activity of Compounds from EU1180-516 to EU1180-614

The afore-referenced compounds were screened in the same assay as EU1180-438 to determine their effects on hGluN1^{FA,TL}/hGluN3A receptors. Selected compounds that inhibited by more than 20% at the concentration at which they were initially evaluated (either 10 or 30 μM) were analyzed further, and a dose-response curve was generated to determine their IC₅₀ values. Table 4 summarizes the biological activity results of these compounds.

EU1180-499, EU1180-508, EU1180-509, EU1880-510, and EU1880-511 were also screened in the same assay as EU1180-438 to determine their effects on GluN2A-D. None of them showed any activity.

TABLE 4
Pharmacological activities toward GluN1/GluN3 receptors

| | | | | hGluN1^{FA,TL}/hGluN3A | |
|---|---|---|---|---|---|
| | | | | (100 × I_{drug}/I_{control} at 30 | IC₅₀ |
| | | R₁ | R₂ | μM drug) | (μM) |
| 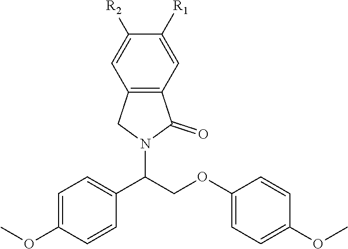 | EU1180-518 EU1180-517 EU1180-516 EU1180-519 EU1180-520 EU1180-521 EU1180-522 | CF₃ H Cl H Br H F | H Cl H Br H F H | 86% 92% 44% 90% 64% 60% 25% | N/A N/A N/A N/A N/A N/A N/A |
| 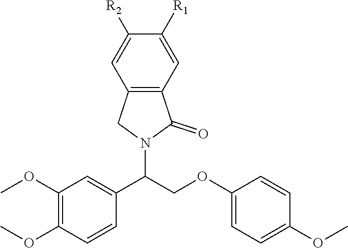 | EU1180-523 EU1180-524 EU1180-525 EU1180-526 EU1180-527 EU1180-528 EU1180-529 | CF₃ H Cl H Br H F | H Cl H Br H F H | 104% 102% 94% 101% 94% 94% 84% | N/A N/A N/A N/A N/A N/A N/A |
| 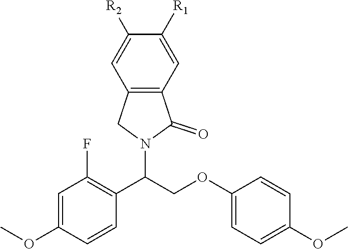 | EU1180-530 EU1180-531 EU1180-532 EU1180-533 EU1180-534 EU1180-535 EU1180-536 EU1180-537 | H CF₃ H Cl H Br H F | H H Cl H Br H F H | 41% 99% 95% 49% 99% 83% 80% 66% | N/A N/A N/A N/A N/A N/A N/A N/A |
| 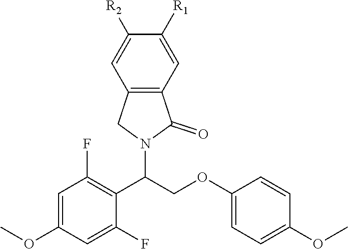 | EU1180-545 EU1180-546 EU1180-547 EU1180-548 EU1180-549 EU1180-550 EU1180-551 EU1180-552 | H CF₃ H Cl H Br H F | H H Cl H Br H F H | 74% 96% 98% 90% 95% 93% 92% 70% | N/A N/A N/A N/A N/A N/A N/A N/A |
| 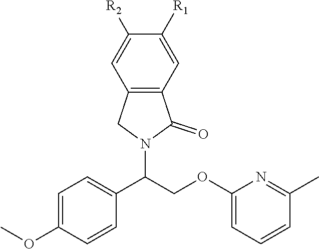 | EU1180-593 EU1180-594 EU1180-595 EU1180-596 EU1180-597 EU1180-598 EU1180-599 | CF₃ H Cl H Br H F | H Cl H Br H F H | 83% 73% 75% 92% 69% 89% 57% | N/A N/A N/A N/A N/A N/A N/A |
| 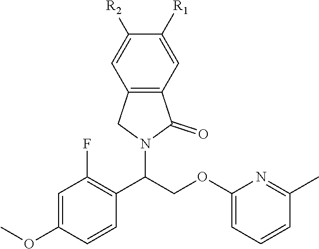 | EU1180-600 EU1180-601 EU1180-602 EU1180-603 EU1180-604 EU1180-605 EU1180-606 EU1180-607 | H H F CF₃ H Cl H Br | H F H H Cl H Br H | 87% 79% 73% 84% 81% 69% 66% 62% | N/A N/A N/A N/A N/A N/A N/A N/A |
| 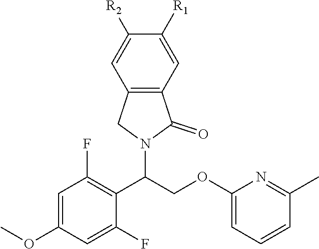 | EU1180-608 EU1180-609 EU1180-610 EU1180-611 EU1180-612 EU1180-613 EU1180-614 | H CF₃ H Cl H H F | H H Cl H Br F H | 70% 81% 76% 57% 79% 89% 57% | N/A N/A N/A 63 N/A N/A N/A |
| 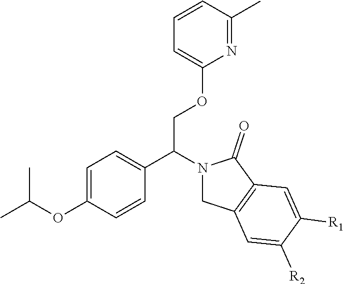 | EU1180-508 EU1180-509 EU1180-510 EU1180-511 EU1180-512 EU1180-513 EU1180-514 EU1180-515 | H Cl H CF₃ F H Br H | H H Cl H H F H Br | 28% @ 10 μM 81% @ 10 μM 94% 94% 36% 87% 93% 100% | 3.93 N/A N/A N/A N/A N/A N/A N/A |
| 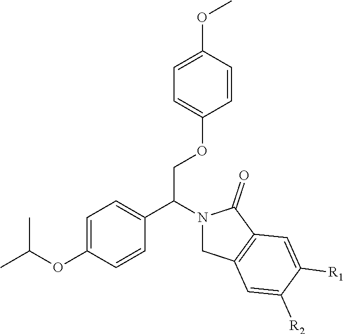 | EU1180-481 EU1180-558 EU1180-564 EU1180-559 EU1180-560 EU1180-561 EU1180-562 EU1180-563 | H CF₃ Cl H F H Br H | H H H Cl H F H Br | 57% @ 10 μM 98% 96% 96% 23% 88% 98% 98% | 3.12 N/A N/A N/A N/A N/A N/A N/A |
| 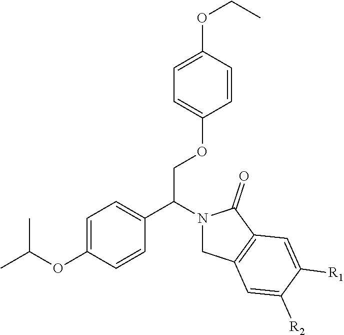 | EU1180-499 EU1180-587 EU1180-588 EU1180-589 EU1180-590 EU1180-591 EU1180-592 | H CF₃ Cl H F H Br | H H H Cl H F H | 67% @ 10 μM 98% 67% 87% 35% 90% 78% | 2.1 N/A N/A N/A 15.7 N/A N/A |
| 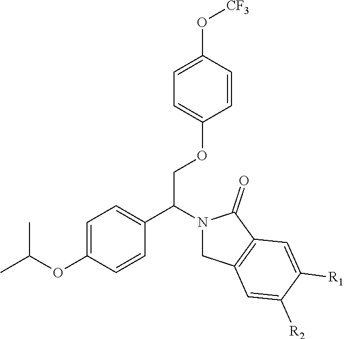 | EU1180-538 EU1180-539 EU1180-540 EU1180-541 EU1180-542 EU1180-544 EU1180-553 EU1180-554 | H CF₃ Cl H F H Br H | H H H Cl H F H Br | 99% 99% 100% 100% 98% 98% 101% 101% | N/A N/A N/A N/A N/A N/A N/A N/A |
| 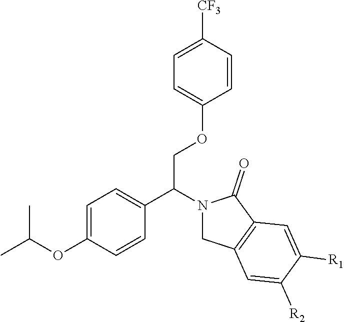 | EU1180-571 EU1180-572 EU1180-573 EU1180-574 EU1180-575 EU1180-576 EU1180-577 EU1180-578 | H CF₃ Cl H F H Br H | H H H Cl H F H Br | 94% 91% 100% 91% 76% 104% 108% 118% | N/A N/A N/A N/A N/A N/A N/A N/A |

N/A: not available

### Example 57. Biological Activity Toward GluN3B

Selected compounds were also screened in the same assay as EU1180-438 to determine their effect on GluN3B. Table 5 summarizes the GluN3B activity of these compounds.

TABLE 5
Pharmacological activities toward GluN3B

| | rat GluN1-4a/GluN3B + CGP ^{a} | |
|---|---|---|
| | (100 × I_{drug}/I_{control}) | IC₅₀ (μM) |
| EU1180-438 (+) | 28% @ 10 μM drug | N/A ^{b} |
| EU1180-438 (−) | 70% @ 10 μM drug | N/A ^{b} |
| EU1180-487 | 42% @ 10 μM drug | 6.49 |
| EU1180-488 | 38% @ 10 μM drug | 3.43 |
| EU1180-489 | 31% @ 10 μM drug | 3.57 |

^{a} The assays were performed in the presence of 0.5 μM CGP-78608.
^{b} N/A: not available

What is claimed is:

1. A compound of Formula I, an enantiomer or diastereomer thereof, or a pharmaceutically acceptable salt thereof, 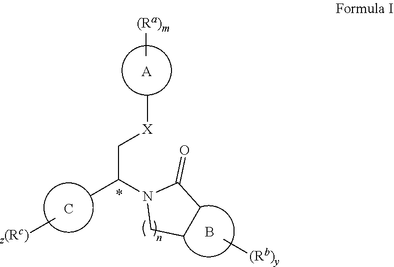
wherein rings A, B and C are individually and independently five- or six-membered aryl, heteroaryl, carbocycle, or heterocycle;
wherein X is O, S, or NH;
wherein n is 1, 2 or 3;
wherein m, y, and z are individually and independently 0, 1, 2, 3, or 4; and
wherein R^{a}, R^{b}, and R^{c}, in each occurrence, are individually and independently selected from the group consisting of hydrogen, deuterium, halogen, nitro, cyano, hydroxyl, trifluoromethoxy, trifluoromethyl, alkylsilyl, formyl, carboxyl, mercapto, sulfamoyl, alkyl, alkyloxy, acyl, acyloxy, amino, alkylamino, acylamino, carbamoyl, N-alkylcarbamoyl, alkylthio, alkylsulfinyl, alkylsulfonyl, alkyloxycarbonyl, N-alkylsulfamoyl, arylalkyl, arylcarbonyl, heteroalkyl, carbocyclyl, heterocyclyl, aryl, and heteroaryl.

2. The compound of claim 1, wherein X is O.

3. The compound of claim 1, wherein rings A, B and C are individually and independently five- or six-membered aryl or heteroaryl.

4. The compound of claim 1, wherein ring A is phenyl.

5. The compound of claim 1, wherein ring A is pyridinyl.

6. The compound of claim 5, wherein ring A is 2-pyridinyl.

7. The compound of claim 1, wherein ring B is phenyl.

8. The compound of claim 1, wherein ring C is phenyl.

9. The compound of claim 1, wherein n is 1 or 2.

10. The compound of claim 1, wherein m is 1.

11. The compound of claim 1, wherein y is 0 or 1.

12. The compound of claim 1, wherein z is 0 or 1.

13. The compound of claim 1, wherein R^{a}, R^{b}, and R^{c}, in each occurrence, are individually and independently selected from the group consisting of hydrogen, deuterium, halogen, nitro, cyano, hydroxyl, trifluoromethoxy, trifluoromethyl, trimethylsilyl, formyl, carboxyl, carbamoyl, mercapto, sulfamoyl, alkyl, alkoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, benzyl, benzoyl, carbocyclyl, aryl, and heterocyclyl.

14. The compound of claim 13, wherein R^{a}, R^{b}, and R^{c}, in each occurrence, are individually and independently selected from the group consisting of hydrogen, halogen, hydroxyl, methyl, trifluoromethyl, trifluoromethoxy, methoxy, ethoxy, and isopropoxy.

15-23. (canceled)

24. The compound of claim 1, selected from the group consisting of:
2-(2-(4-methoxyphenoxy)-1-(4-methoxyphenyl)ethyl)isoindolin-1-one,
2-(1-(4-isopropoxyphenyl)-2-(4-methoxyphenoxy)ethyl)isoindolin-1-one,
2-(2-(4-methoxyphenoxy)-1-phenyl-ethyl)isoindolin-1-one,
2-(2-(4-methoxyphenoxy)-1-phenylethyl)-3,4-dihydroisoquinolin-1(2H)-one,
2-(2-(4-methoxyphenoxy)-1-(4-methoxyphenyl)ethyl)-3,4-dihydroisoquinolin-1(2H)-one,
2-(2-(4-ethoxyphenoxy)-1-(4-isopropoxyphenyl)ethyl)isoindolin-1-one,
2-(1-(4-isopropoxyphenyl)-2-((6-methylpyridin-2-yl)oxy)ethyl)isoindolin-1-one,
6-fluoro-2-(1-(4-isopropoxyphenyl)-2-((6-methylpyridin-2-yl)oxy)ethyl)isoindolin-1-one,
6-chloro-2-(2-(4-methoxyphenoxy)-1-(4-methoxyphenyl)ethyl)isoindolin-1-one,
6-bromo-2-(2-(4-methoxyphenoxy)-1-(4-methoxyphenyl)ethyl)isoindolin-1-one,
5-fluoro-2-(2-(4-methoxyphenoxy)-1-(4-methoxyphenyl)ethyl)isoindolin-1-one,
6-fluoro-2-(2-(4-methoxyphenoxy)-1-(4-methoxyphenyl)ethyl)isoindolin-1-one,
2-(1-(2-fluoro-4-methoxyphenyl)-2-(4-methoxyphenoxy)ethyl)isoindolin-1-one,
6-chloro-2-(1-(2-fluoro-4-methoxyphenyl)-2-(4-methoxyphenoxy)ethyl)isoindolin-1-one,
6-fluoro-2-(1-(2-fluoro-4-methoxyphenyl)-2-(4-methoxyphenoxy)ethyl)isoindolin-1-one,
6-fluoro-2-(1-(4-isopropoxyphenyl)-2-(4-methoxyphenoxy)ethyl)isoindolin-1-one,
6-chloro-2-(2-(4-ethoxyphenoxy)-1-(4-isopropoxyphenyl)ethyl)isoindolin-1-one,
6-fluoro-2-(2-(4-ethoxyphenoxy)-1-(4-isopropoxyphenyl)ethyl)isoindolin-1-one,
6-bromo-2-(1-(4-methoxyphenyl)-2-((6-methylpyridin-2-yl)oxy)ethyl)isoindolin-1-one,
6-fluoro-2-(1-(4-methoxyphenyl)-2-((6-methylpyridin-2-yl)oxy)ethyl)isoindolin-1-one,
6-chloro-2-(1-(2-fluoro-4-methoxyphenyl)-2-((6-methylpyridin-2-yl)oxy)ethyl)isoindolin-1-one,
5-bromo-2-(1-(2-fluoro-4-methoxyphenyl)-2-((6-methylpyridin-2-yl)oxy)ethyl)isoindolin-1-on,
6-bromo-2-(1-(2-fluoro-4-methoxyphenyl)-2-((6-methylpyridin-2-yl)oxy)ethyl)isoindolin-1-one,
6-chloro-2-(1-(2,6-difluoro-4-methoxyphenyl)-2-((6-methylpyridin-2-yl)oxy)ethyl)isoindolin-1-one, and
2-(1-(2,6-difluoro-4-methoxyphenyl)-2-((6-methylpyridin-2-yl)oxy)ethyl)-6-fluoroisoindolin-1-one.

25-27. (canceled)

28. A pharmaceutical formulation, comprising the compound of claim 1 and a pharmaceutically acceptable carrier.

29. The pharmaceutical formulation of claim 28, wherein the pharmaceutical formulation is in the form of tablet, capsule, pill, gel, cream, granule, solution, suspension, emulsion, or nanoparticulate formulation.

30. The pharmaceutical formulation of claim 28, wherein the pharmaceutical formulation is an oral formulation.

31. A method of treating a neurological condition or disorder in a subject in need thereof, comprising administering an effective amount of the compound of claim 1 to the subject.

32. The method of claim 31, wherein the compound is administered orally.

33. The method of claim 31, wherein the neurological condition or disorder is selected from the group consisting of depression, bipolar disorder, attention-deficit disorder, schizophrenia, anxiety, psychoses, epilepsies, dystonia, Huntingtin's disease, L-DOPA-induced dyskinesias or dyskinesias resulting from medication, substance abuse/addiction or predisposition to substance abuse/addiction, pain disorders, neurodegenerative diseases, motor retraining after acute injury, and acute neuronal or glial injuries.

34. The method of claim 33, wherein the neurodegenerative diseases comprise Alzheimer's disease, Parkinson's disease, and frontal lobe dementia.

35. (canceled)
